(19) United States
(12) Patent Application Publication
(10) Pub. No.: US 2024/0352453 A1
(43) Pub. Date: Oct. 24, 2024

(54) GENOME EDITING COMPOSITIONS AND METHODS FOR TREATMENT OF RETINOPATHY

(71) Applicant: Prime Medicine, Inc., Cambridge, MA (US)

(72) Inventors: Wei Hsi YEH, Cambridge, MA (US); Dewi HARJANTO, Brookline, MA (US)

(21) Appl. No.: 18/584,538

(22) Filed: Feb. 22, 2024

Related U.S. Application Data
(63) Continuation of application No. PCT/US2022/041430, filed on Aug. 24, 2022.
(60) Provisional application No. 63/236,544, filed on Aug. 24, 2021.

(51) Int. Cl. C12N 15/11; C12N 9/12; C12N 9/22

(52) U.S. Cl. CPC C12N 15/111; C12N 9/1276; C12N 9/22; C12N 2310/20

(57) ABSTRACT

Provided herein are compositions and methods of using prime editing systems comprising prime editors and prime editing guide RNAs for treatment of genetic disorders.

### CROSS-REFERENCE

This application is a continuation of International Application No. PCT/US2022/041430, filed Aug. 24, 2022, which claims the benefit of U.S. Provisional Application No. 63/236,544, filed Aug. 24, 2021, each of which applications are incorporated herein by reference in their entirety.

### SEQUENCE LISTING

The instant application contains a Sequence Listing which has been submitted electronically in XML format and is hereby incorporated by reference in its entirety. Said XML copy, created on Oct. 26, 2022, is named 59761-744.601_updated.xml and is 8,509,379 bytes in size.

### BACKGROUND

Non-syndromic retinitis pigmentosa (nsRP) is an autosomal recessive disorder that involves progressive loss of photoreceptors that leads to night blindness and constricted visual field, followed by decline of peripheral and foveal cone populations, eventually resulting in loss of central vision and severe visual impairment in patients.

Usher syndrome is an autosomal recessive disorder involving dual impairment of the visual and audiovestibular systems and is the most common cause of deaf-blindness. Patients with Usher syndrome often have congenital sensorineural hearing loss with or without vestibular dysfunction, and visual loss in the form of retinitis pigmentosa (RP). Usher syndrome type 2 is the most common form of Usher syndrome accounting for more than 50% of all cases and exhibits a phenotype with moderate-to-severe hearing defects and later onset of RP.

Mutations in the USH2A gene are a leading cause of retinopathy including nsRP and Usher syndrome type 2. The human USH2A gene is located on the long arm of chromosome 1q41, and codes for multiple usherin isoforms. The usherin isoforms include a short isoform a containing 21 exons leading to a 1546-aa secreted protein, and a very large isoform b with 51 additional exons that is predominant in the retina and the cochlear, giving rise to a 5202-aa matrix protein with a predicted total molecular weight of 570 kDa. The isoform b comprises an intracellular region which interacts with the Usher protein network, a short transmembrane domain and a very long extracellular domain with several motifs associated with extracellular matrix proteins, e.g., laminin and fibronectin repeats. Pathogenic mutations in the USH2A gene include frameshifts and missense mutations that may result in reduced or non-functional usherin protein, and nonsense mutations that results in truncated usherin protein. While patients with Usher type 2 may benefit from hearing aids or cochlear implants to correct their hearing defects, no approved treatments are available for the USH2A-retinopathy.

This disclosure provides prime editing methods and compositions for correcting mutations associated with USH2A-retinopathy and treatment for USH2A-retinopathy such as nsRP and Usher syndrome.

### SUMMARY OF THE DISCLOSURE

Provided herein, in some embodiments, are methods and compositions for prime editing of alterations in a target sequence in a target gene, for example, an USH2A gene. The target USH2A gene may comprise double stranded DNA. As exemplified in FIG. 1, in an embodiment, the target gene is edited by prime editing.

Without wishing to be bound by any particular theory, the prime editing process may search specific targets and edit endogenous sequences in a target gene, e.g., the USH2A gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex may then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target USH2A gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target USH2A gene.

Accordingly, disclosed herein are prime editing guide RNAs (PEgRNAs) comprising: (a) a spacer that is complementary to a search target sequence on a first strand of an USH2a gene. wherein the spacer comprises at its 3′ end a sequence selected from the list consisting of SEQ ID NOs: 618, 469, 1, 1556, 950, 1673, 1733, 1773, 1823, 3102, 3561, 400, 6027, 296, 2099, 7240, 1102, 73, 7459, 1, 55, 141, 296, 382, 469, 513, 734, 763, 643, 932, 1059, 1095, 1164, 1321, 1412, 1895, 2005, 2087, 4496, 4541, 5822, 5864, 5991, and 7084; (b) a gRNA core capable of binding to a Cas9 protein; (c) an extension arm comprising: (i) an editing template that comprises a region of complementarity to an editing target sequence on a second strand of the USH2a gene, and (ii) a primer binding site that comprises at its 5′ end a sequence that is a reverse complement of nucleotides 10-14 of the selected spacer sequence; wherein the first strand and second strand are complementary to each other and wherein the editing target sequence on the second strand comprises or is complementary to a portion of the USH2a gene comprising a c.2276 G>T substitution or a c.2299de1G mutation and wherein the editing template encodes or comprises the wild type amino acid sequence of USH-2a protein at the c.2276 G>T substitution and/or c.2299de1G mutation sites.

Also disclosed herein are prime editing guide RNAs (PEgR NAs), or nucleic acids encoding the PEgRNAs, comprising: (a) a spacer comprising at its 3′ end a PEgRNA Spacer sequence selected from any one of Tables 1-44; (b) a gRNA core capable of binding to a Cas9 protein, and (c) an extension arm comprising: (i) an editing template comprising at its 3′ end an RTT sequence selected from the same Table as the PEgRNA Spacer sequence, and (ii) a primer binding site (PBS) comprising at its 5′ end a PBS sequence selected from the same Table as the PEgRNA Spacer sequence.

Some exemplary prime editing guide RNAs (PEgRNAs), or a nucleic acids encoding the PEgRNAs, disclosed herein comprise: (a) a spacer comprising at its 3′ end SEQ ID NO: 5864; (b) a gRNA core capable of binding to a Cas9 protein, and (c) an extension arm comprising: (i) an editing template comprising at its 3′ end SEQ ID NOs: 5869, and (ii) a primer binding site (PBS) comprising at its 5′ end a sequence that is a reverse complement of nucleotides 11-14 of SEQ ID NO: 5864. In some embodiments, the spacer comprises at its 3′ end SEQ ID NO: 5865, 5866, 38, 5867, or 5868. In some embodiments, the spacer comprises at its 3′ end SEQ ID NO: 38. In some embodiments, the editing template comprises at its 3′ end SEQ ID NO: 5869, 5870, 5871, 5872, 5873, 5874, 5875, 5876, 5877, 5878, 5879, 5880, 5881, 5882, 5883, or 5884. In some embodiments, the PBS comprises sequence number 5849, 5850, 5851, 5852, 5853, 5854, 5855, 5856, 5857, 5858, 5859, 5860, 5861, 5862, or 5863. Also disclosed herein are prime editing systems comprising such PEgRNAs or nucleic acids and a ngRNA or nucleic acid encoding the ngRNA comprising: (a) a spacer comprising at its 3′ end a sequence corresponding to nucleotides 4-20 of SEQ ID NO: 4, 58, 1898, 4508, 4509, 4510, 4511, 4512, 2008, 4634, 4635, 1676, 4519, 646, 617, 4522, 3564, 1736, 4640, 2090, 380, 1098, 4525, or 1324; and (b) an ngRNA core capable of binding a Cas9 protein.

Some prime editing guide RNAs (PEgRNAs), or a nucleic acids encoding the PEgRNAs, disclosed herein comprise: (a) a spacer comprising at its 3′ end SEQ ID NO: 4541: (b) a gRNA core capable of binding to a Cas9 protein, and (c) an extension atm comprising: (i) an editing template comprising at its 3′ end any one of SEQ ID NOs: 4546, 4549, 4550, or 4551, and (ii) a primer binding site (PBS) comprising at its 5′ end a sequence that is a reverse complement of nucleotides 11-14 of SEQ ID NO: 4541. In some embodiments, the spacer comprises at its 3′ end SEQ ID NO: 4542, 4543, 48, 4544, or 4545. In some embodiments, the spacer comprises at its 3′ end SEQ ID NO: 48. In some embodiments, the editing template comprises at its 3′ end SEQ ID NO: 4546, 4547, 4548, 4511, 4553, 4557, 4561, 4565, 4569, 4573, 4577, 4581, 4585, 4589, 4593, 4597, 4601, 4605, 4609, 4613, 4617, 4621, 4625, or 4629. In some embodiments, the editing template comprises at its 3′ end SEQ ID NO: 4549, 4550, 4551, 4552, 4554, 4555, 4556, 4558, 4559, 4560, 4562, 4563, 4564, 4566, 4567, 4568, 4570, 4571, 4572, 4574, 4575, 4576, 4578, 4579, 4580, 4582, 4583, 4584, 4586, 4587, 4588, 4590, 4591, 4592, 4594, 4595, 4596, 4598, 4599, 4600, 4602, 4603, 4604, 4606, 4607, 4608, 4610, 4611, 4612, 4614, 4615, 4616, 4618, 4619, 4620, 4622, 4623, 4624, 4626, 4627, 4628, 4630, or 4631, and wherein the editing template encodes a PAM silencing mutation. In some embodiments, the PBS comprises sequence number 4526, 4527, 4528, 4529, 4530, 4531, 4532, 4533, 4534, 4535, 4536, 4537, 4538, 4539, or 4540. Also disclosed herein are prime editing systems comprising such PEgRNAs or nucleic acids and a ngRNA or nucleic acid encoding the ngRNA: (a) a spacer comprising at its 3′ end a sequence corresponding to nucleotides 4-20 of SEQ ID NO: 4, 58, 1898, 4509, 4510, 4549, 4511, 4550, 4551, 2008, 4634, 4635, 1676, 4519, 646, 4636, 4522, 4637, 4638, 4639, 3564, 1736, 4640, 2090, 380, 1098, or 1324; and (b) an ngRNA core capable of binding a Cas9 protein.

Some exemplary prime editing guide RNAs (PEgRNAs), or a nucleic acids encoding the PEgRNAs, disclosed herein comprise: (a) a spacer comprising at its 3′ end SEQ ID NO: 2087; (b) a gRNA core capable of binding to a Cas9 protein, and (c) an extension arm comprising: (i) an editing template comprising at its 3′ end any one of SEQ ID NOs: 2093, 2096, 2097, or 2098. and (ii) a primer binding site (PBS) comprising at its 5′ end a sequence that is a reverse complement of nucleotides 11-14 of SEQ ID NO: 2087. In some embodiments, the spacer comprises at its 3′ end SEQ ID NO: 2088, 2089, 2090, 2091, or 2092. In some embodiments, the spacer comprises at its 3′ end SEQ ID NO: 2090. In some embodiments, the editing template comprises at its 3′ end SEQ ID NO: 2093, 2094, 2095, 2099, 2103, 2107, 30, 2114, 2118, 2122, 2126, 2130, 2134, 2138, 2142, 2146, 2150, 2154, 2158, 2162, 2166, 2170, 2174, 2178, 2182, 2186, or 2190. In some embodiments, the editing template comprises at its 3′ end SEQ ID NO: 2096, 2097, 2098, 2100, 2101, 2102, 2104, 2105, 2106, 2108, 2109, 2110, 2111, 2112, 2113, 2115, 2116, 2117, 2119, 2120, 2121, 2123, 2124, 2125, 2127, 2128, 2129, 2131, 2132, 2133, 2135, 2136, 2137, 2139, 2140, 2141, 2143, 2144, 2145, 2147, 2148, 2149, 2151, 2152, 2153, 2155, 2156, 2157, 2159, 2160, 2161, 2163, 2164, 2165, 2167, 2168, 2169, 2171, 2172, 2173, 2175, 2176, 2177, 2179, 2180, 2181, 2183, 2184, 2185, 2187, 2188, or 2189, and wherein the editing template encodes a PAM silencing mutation. In some embodiments, the PBS comprises sequence number 2072, 2073, 2074, 2075, 2076, 2077, 2078, 2079, 2080, 2081, 2082, 2083, 2084, 2085, or 2086. Also disclosed herein are prime editing systems comprising such PEgRNAs or nucleic acids and a ngRNA or nucleic acid encoding the ngRNA; (a) a spacer comprising at its 3′ end a sequence corresponding to nucleotides 4-20 of SEQ ID NO: 30, 31, 33, 34, 138, 35, 36, 139, 37, 38, 39, 41, 43, 44, 140, 48, 49, 51, 52, 53, or 54; and (b) an ngRNA core capable of binding a Cas9 protein.

Some exemplary prime editing guide RNAs (PEgRNAs), or a nucleic acids encoding the PEgRNAs, disclosed herein comprise: (a) a spacer comprising at its 3′ end SEQ ID NO: 2005; (b) a gRNA core capable of binding to a Cas9 protein, and (c) an extension arm comprising: (i) an editing template comprising at its 3′ end SEQ ID NO: 2011, and (ii) a primer binding site (PBS) comprising at its 5′ end a sequence that is a reverse complement of nucleotides 11-14 of SEQ ID NO: 2005. In some embodiments, the spacer comprises at its 3′ end SEQ ID NO: 2006, 2007, 2008, 2009, or 2010. In some embodiments, the spacer comprises at its 3′ end SEQ ID NO: 2008. In some embodiments, the editing template comprises at its 3′ end SEQ ID NO: 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, or 2039. In some embodiments, the PBS comprises sequence number 1990, 1991, 1992, 1993, 1994, 1995, 1996, 1997, 1998, 1999, 2000, 2001, 2002, 2003, or 2004. Also disclosed herein are prime editing systems comprising such PEgRNAs or nucleic acids and a ngRNA or nucleic acid encoding the ngRNA: (a) a spacer comprising at its 3′ end a sequence corresponding to nucleotides 4-20 of SEQ ID NO: 2039, 30, 31, 33, 34, 138, 35, 139, 37, 38, 39, 41, 43, 44, 140, 48, 49, 51, 52, 53, or 54; and (b) an ngRNA core capable of binding a Cas9 protein.

Some exemplary prime editing guide RNAs (PEgRNAs), or a nucleic acids encoding the PEgRNAs. disclosed herein comprise: (a) a spacer comprising at its 3′ end SEQ ID NO: 1895; (b) a gRNA core capable of binding to a Cas9 protein, and (c) an extension arm comprising: (i) an editing template comprising at its 3′ end any one of SEQ ID NOs: 1901 or 1904, and (ii) a primer binding site (PBS) comprising at its 5′ end a sequence that is a reverse complement of nucleotides 11-14 of SEQ ID NO: 1895. In some embodiments, the spacer comprises at its 3′ end SEQ ID NO: 1896, 1897, 1898, 1899, or 1900. In some embodiments, the spacer comprises at its 3′ end SEQ ID NO: 1898. In some embodiments, the editing template comprises at its 3′ end SEQ ID NO: 1901, 1902, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, or 1957. In some embodiments, the editing template comprises at its 3′ end SEQ ID NO: 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, or 1956. and wherein the editing template encodes a PAM silencing mutation. In some embodiments, the PBS comprises sequence number 1880, 1881, 1882, 1883, 1884, 1885, 1886, 1887, 1888, 1889, 1890, 1891, 1892, 1893, or 1894. Also disclosed herein are prime editing systems comprising such PEgRNAs or nucleic acids and a ngRNA or nucleic acid encoding the ngRNA: (a) a spacer comprising at its 3′ end a sequence corresponding to nucleotides 4-20 of SEQ ID NO: 30, 31, 34, 138, 35, 139, 37, 38, 39, 41, 43, 44, 140, 48, 49, 51, 52, 53, or 54; and (b) an ngRNA core capable of binding a Cas9 protein.

Some exemplary prime editing guide RNAs (PEgRNAs), or a nucleic acids encoding the PEgRNA s, disclosed herein comprise: (a) a spacer comprising at its 3′ end SEQ ID NO: 2099; (b) a gRNA core capable of binding to a Cas9 protein, and (c) an extension arm comprising: (i) an editing template comprising at its 3′ end any one of SEQ ID NOs: 7108, 7110, 7111, or 7112. and (ii) a primer binding site (PBS) comprising at its 5% end a sequence that is a reverse complement of nucleotides 11-14 of SEQ ID NO: 2099. In some embodiments, the spacer comprises at its 3′ end SEQ ID NO: 2103, 2107, 30, 2114, or 2118. In some embodiments, the spacer comprises at its 3′ end SEQ ID NO: 30. In some embodiments, the editing template comprises at its 3′ end SEQ ID NO: 7108, 7109, 2087, 2088, 2089, 2090, 2091, 2092, 7126, 7130, 7134, 7138, 7142, 7146, 7150, 7154, 7158, 7162, 7166, 7170, 7174, 7178, 7182, 7186, 7190, or 7194. In some embodiments, the editing template comprises at its 3′ end SEQ ID NO: 7110, 7111, 7112, 7113, 7114, 7115, 7116, 7117, 7118, 7119, 7120, 7121, 7122, 7123, 7124, 7125, 7127, 7128, 7129, 7131, 7133, 7132, 7135, 7136, 7137, 7139, 7140, 7141, 7143, 7144, 7145, 7147, 7148, 7149, 7151, 7152, 7153, 7155, 7156, 7157, 7159, 7160, 7161, 7163, 7164, 7165, 7167, 7169, 7168, 7171, 7172, 7173, 7175, 7176, 7177, 7179, 7180, 7181, 7183, 7184, 7185, 7187, 7188, 7189, 7191, 7192, 7193, 7195, or 7196, and wherein the editing template encodes a PAM silencing mutation. In some embodiments, the PBS comprises sequence number 7094, 7095, 7096, 7097, 7098, 7099, 7100, 7101, 7102, 322, 7103, 7104, 7105, 7106, or 7107. Also disclosed herein are prime editing systems comprising such PEgRNAs or nucleic acids and a ngRNA or nucleic acid encoding the ngRNA: (a) a spacer comprising at its 3′ end a sequence corresponding to nucleotides 4-20 of SEQ ID NO: 4, 5971, 667, 1898, 1079, 4510, 1657, 2008, 5972, 4634, 4635, 1676, 646, 7197, 379, 7198, 7199, 4639, 3564, 5973, 1736, 4640, 2090, 380, 1098, 1324, or 5975; and (b) an ngRNA core capable of binding a Cas9 protein.

Some exemplary prime editing guide RNAs (PEgRNAs), or a nucleic acids encoding the PEgRNAs, disclosed herein comprise: (a) a spacer comprising at its 3′ end SEQ ID NO: 6027; (b) a gRNA core capable of binding to a Cas9 protein, and (c) an extension arm comprising: (i) an editing template comprising at its 3′ end any one of SEQ ID NOs: 6032, 6033, 6038, or 6041, and (ii) a primer binding site (PBS) comprising at its 5′ end a sequence that is a reverse complement of nucleotides 11-14 of SEQ ID NO: 6027. In some embodiments, the spacer comprises at its 3′ end SEQ ID NO: 6028, 6029, 33, 6030, or 6031. In some embodiments, the spacer comprises at its 3′ end SEQ ID NO: 33. In some embodiments, the editing template comprises at its 3′ end SEQ ID NO: 6032, 6034, 6036, 6039, 6043, 6047, 6051, 6055, 6059, 6063, 6067, 6071, 6075, 6079, 6083, 6087, 6091, 6099, 6095, 6103, 6107, 6111, 6115, 6119, 6123, 6127, or 6131. In some embodiments, the editing template comprises at its 3′ end SEQ ID NO: 6033, 6035, 6037, 6038, 6040, 6041, 6042, 6044, 6045, 6046, 6048, 6049, 6050, 6052, 6053, 6054, 6056, 6057, 6058, 6060, 6061, 6062, 6064, 6065, 6066, 6068, 6069, 6070, 6072, 6073, 6074, 6076, 6077, 6078, 6080, 6081, 6082, 6084, 6085, 6086, 6088, 6089, 6090, 6092, 6093, 6094, 6096, 6097, 6098, 6100, 6101, 6102, 6104, 6105, 6106, 6108, 6109, 6110, 6112, 6113, 6114, 6116, 6117, 6118, 6120, 6121, 6122, 6124, 6125, 6126, 6128, 6129, 6130, 6132, or 6133, and wherein the editing template encodes a PAM silencing mutation. In some embodiments, the PBS comprises sequence number 6013, 6014, 6015, 6016, 6017, 6018, 6019, 6020, 6021, 6022, 324, 6023, 6024, 6025, or 6026. Also disclosed herein are prime editing systems comprising such PEgRNAs or nucleic acids and a ngRNA or nucleic acid encoding the ngRNA: (a) a spacer comprising at its 3′ end a sequence corresponding to nucleotides 4-20 of SEQ ID NO: 4, 5971, 4510, 6136, 2008, 6137, 6138, 5972, 4634, 4635, 1676, 646, 4639, 3564, 5973, 1736, 4640, 2090, 380, 1098, 1324, or 5975; and (b) an ngRNA core capable of binding a Cas9 protein.

Any of the PEgRNA disclosed herein can comprise, from 5′ to 3′. the spacer, the gRNA core, the RTT, and the PBS. In some embodiments, the spacer, the gRNA core, the RTT, and the PBS form a contiguous sequence in a single molecule. In some embodiments, the editing template has a length of 44 nucleotides or less, in some embodiments, the editing template has a length of 34 nucleotides or less. In some embodiments, the editing template has a length of 22 nucleotides or less. In some embodiments, the 3′ end of the editing template is adjacent to the 5′ end of the PBS. Some embodiments further comprise 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. Some embodiments further comprise 3′ mN*mN*mN*N and 5′mN*mN*mN* modifications, where m indicates that the nucleotide contains a 2′-O-Me modification and a * indicates the presence of a phosphorothioate bond.

Any of the prime editing systems disclosed herein can further comprise (c) a prime editor comprising a Cas9 nickase having a nuclease inactivating mutation in the HNH domain, or a nucleic acid encoding the Cas9 nickase, and a reverse transcriptase, or a nuclei acid encoding the reverse transcriptase. In some embodiments, the prime editor is a fusion protein.

Any of the prime editing systems disclosed herein can further comprise (c) an N-terminal extein comprising an N-terminal fragment of a prime editor fusion protein and an N-intein or a polynucleotide encoding the N-terminal extein; and (d) a C-terminal extein comprising a C-terminal fragment of the prime editor fusion protein and a C-intein, or a polynucleotide encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase and a reverse transcriptase (RT) domain.

Also disclosed herein are LNPs, viral vectors and populations of viral particles for delivering the PEgRNA and prime editing systems disclosed herein.

Also disclosed herein are methods for editing an USH2a gene using the PEgRNA and prime editing systems disclosed herein.

Also disclosed herein are methods for treating Usher syndrome and/or nsRP in subjects in need thereof with the PEgRNA, prime editing systems, LNPs, and viral vectors disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of a prime editing guide RNA (PEgRNA) binding to a double stranded target DNA sequence.

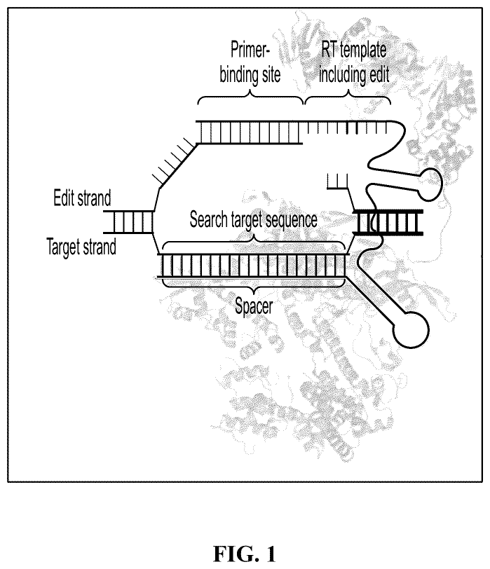

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

### DETAILED DESCRIPTION OF THE DISCLOSURE

Provided herein, in some embodiments, are compositions and methods to edit the target gene USH2A with prime editing. In certain embodiments, provided herein are compositions and methods for correction of mutations in the USH2A gene associated with non-syndromic retinitis pigmentosa (nsRP). In certain embodiments, provided herein are compositions and methods for correction of mutations in the USH2A gene associated with Usher syndrome. Compositions provided herein can comprise prime editors (PEs) that may use engineered guide polynucleotides, e.g., prime editing guide RNAs (PEgRNAs), that can direct PEs to specific DNA targets and can encode DNA edits on the target gene USH2A that serve a variety of functions, including direct correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical value means a range of values that fall within 10% greater than or 10% less than the value. For example, about x means x+(10% * x).

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term “primary cell” means a cell isolated from an organism, e.g., a mammal, which is grown in tissue culture (i.e., in vitro) for the first time before subdivision and transfer to a subculture. In some non-limiting examples, mammalian cells, including primary cells and stem cells, can be modified through introduction of one or more polynucleotides, polypeptide, and/or prime editing compositions (e.g., through transfection, transduction, electroporation and the like) and further passaged. Such modified mammalian primary cells include retinal cells (photoreceptors, retinal pigment epithelium cells, epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a stem cell. In some embodiments, the cell is a human progenitor cell. In some embodiments, the cell is a pluripotent cell (e.g., a pluripotent stem cell). In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is an embryonic stem cell (ESC). In some embodiments, the cell is an induced human pluripotent stem cell (iPSC). In some embodiments, the cell is a human stem cell. In some embodiments, the cell is a human embryonic stem cell.

In some embodiments, a cell is not isolated from an organism but forms part of a tissue or organ of an organism, e.g., a mammal. In some non-limiting examples, mammalian cells include epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a sensory ciliated cell. In some embodiments, the cell is a retinal cell. In some embodiments, the cell is a photoreceptor cell. In some embodiments, the cell is a rod cell. In some embodiments, the cell is a cone cell. In some embodiments, the cell is a hair cell. In some embodiments, the cell is a post-natal hair cell. In some embodiments, the cell is a human sensory ciliated cell. In some embodiments, the cell is a human retinal cell. In some embodiments, the cell is a human photoreceptor cell. In some embodiments, the cell is a human rod cell. In some embodiments, the cell is a human cone cell. In some embodiments, the cell is a human hair cell. In some embodiments, the cell is a human post-natal hair cell

In some embodiments, the cell is a differentiated cell. In some embodiments, cell is a fibroblast. In some embodiments, the cell is differentiated from an induced pluripotent stem cell (iPSC). In some embodiments, the cell is differentiated from an embryonic stem cell (ESC). In some embodiments, the cell is differentiated from a keratinocyte. In some embodiments, the cell is differentiated from a human keratinocyte. In some embodiments, the cell is differentiated from a human induced pluripotent stem cell (hiPSC). In some embodiments, the cell is differentiated from a human ESC. In some embodiments, the cell is a differentiated retinal cell. In some embodiments, the cell is a photoreceptor cell, a rod cell, or a cone cell derived from an iPSC. In some embodiments, the cell is a hair cell derived from an iPSC. In some embodiments, the cell is a photoreceptor cell, a rod cell, or a cone cell derived from a hiPSC. In some embodiments, the cell is a hair cell derived from a hiPSC. In some embodiments, the cell is a photoreceptor cell, a rod cell, or a cone cell derived from an ESC. In some embodiments, the cell is a hair cell derived from an ESC. In some embodiments, the cell is a differentiated human cell. In some embodiments, the cell is a human fibroblast. In some embodiments, a human retinal cell is differentiated from a human iPSC or human ESC. In some embodiments, a human hair cell is differentiated from a human iPSC or human ESC.

In some embodiments, the cell comprises a prime editor, a PEgRNA, or a prime editing composition disclosed herein. In some embodiments, the cell further comprises an ngRNA. In some embodiments, the cell is from a human subject. In some embodiments, the human subject has a disease or condition, or is at a risk of developing a disease or a condition associated with a mutation to be corrected by prime editing, for example, nsRP or Usher syndrome. In some embodiments, the cell is from a human subject, and comprises a prime editor or a prime editing composition for correction of the mutation. In some embodiments, the cell is from the human subject and the mutation has been edited or corrected by prime editing. In some embodiments, the cell is in a human subject, and comprises a prime editor, a PEgRNA, or a prime editing composition for correction of the mutation. In some embodiments, the cell is from the human subject and the mutation has been edited or corrected by prime editing. In some embodiments, the cell is in a subject, e.g., a human subject. In some embodiments, the cell is obtained from a subject prior to editing. For example, the cell is obtained from a subject having a mutation in the USH2A gene.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of a retrovirus (e.g., a Moloney murine leukemia virus) or a variant of the retrovirus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, primer binding site or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 7576, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or a uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion or a region of two polynucleotide molecules. In some embodiments, the portion or the region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example a prime editing composition comprising a construct, that can be sufficient to result in a desired activity upon introduction into a subject as disclosed herein. An effective amount of the prime editing compositions can be provided to the target gene or cell, whether the cell is ex vivo or in vivo. An effective amount can be the amount to induce, for example, at least about a 2-fold change (increase or decrease) or more in the amount of target nucleic acid modulation (e.g., expression of a USH2A gene to produce functional usherin protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target USH2A gene to produce functional usherin protein).

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

In some embodiments, an effective amount can be an amount to induce, when administered to a population of cells, at least about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in the number of cells that have an intended nucleotide edit, for example, a nucleotide edit that corrects a c.2299delG mutation or a nucleotide edit that corrects a c.2276G->T mutation in the USH2A gene. In some embodiments, an effective amount can be an amount to induce, when administered to a population of cells, a certain percentage of the population of cells to have a correction of a mutation. For example, in some embodiments, an effective amount can be an amount to induce, when administered to or introduced to a population of cells, installation of an intended nucleotide edit, a nucleotide edit that corrects a c.2299delG mutation or a nucleotide edit that corrects a c.2276G->T mutation in the USH2A gene, in at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

The terms “USH2A”, “Usherin”, “RP39”, “USH2”, “Usher Syndrome Type IIa Protein”, “DJ1111A8.1”, or “Usher Syndrome 2A” as used herein refers to the gene that encodes multiple isoforms of the polypeptide Usherin. The usherin isoforms include a short isoform a containing 21 exons leading to a 1546-aa secreted protein, and a very large isoform b with 51 additional exons that is predominant in the retina and the cochlear, giving rise to a 5202-aa matrix protein with a predicted total molecular weight of 570 kDa. The Usherin isoforms comprises an extracellular domain that is proposed to interact with basement membrane collagen IV and fibronectin via laminin domains (see, e.g., Maerker et al., 2008; Reiners et al., Hum Mol Genet. 2005 Dec. 15; 14(24): 3933-43). Usherin also interacts with other proteins of USH1 and USH2 complex to form Usher networks (Human Molecular Genetics, 26, 1157-1172). Mutations in USH2A are associated with Usher syndrome type II and non-syndromic retinitis pigmentosa (NSRP). The nucleotide and amino acid sequence of USH2A is known and may be found in, for example, GenBank Accession Nos. NM_206933, NM_007123, NM_021408, NP_009054, NP_996816, or NP_067383, the entire contents of each of which are incorporated herein by reference.

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is downstream of a specific PAM sequence on the PAM strand of the double stranded target DNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and which encodes a single strand of DNA. The editing template may comprise a region of complementarity to the PAM strand (i.e., the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit position(s). As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template may encode the wild-type or non-disease associated gene sequence (or its complement if the edit strand is the antisense strand of a gene). In some embodiments, the editing template may encode the wild-type or non-disease associated protein, but contain one or more synonymous mutations relative to the wild-type or non-disease associated protein coding region. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer).

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to an endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template may be referred to as an “editing target sequence”. Accordingly, in some embodiments, the newly synthesized single-stranded DNA has identity or substantial identity to a sequence in the editing target sequence, except for one or more insertions, deletions, or substitutions intended nucleotide edit positions. In some embodiments, the editing template comprises at least 4 contiguous nucleotides of complementarity with the edit strand wherein the at least 4 nucleotides contiguous are located upstream of the 5′ most edit in the editing template.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

In some embodiments, the DNA polymerases can be wild type polymerases from eukaryotic, prokaryotic, archaeal, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcusfuriosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLDI DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a RevI DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcusfuriosus. In some embodiments, the DNA polymerase is a pol II type DNA polymerase. For example, in some embodiments, the DNA polymerase is a homolog of P.furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KODI, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcusfuriosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MMLV) RT; human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT, a functional mutant, a functional variant, or a functional fragment thereof. An exemplary sequence of a reference M-MLV RT is provided in SEQ ID NO: 7597.

In some embodiments, the prime editor comprises a reference M-MLV RT, a functional mutant, a functional variant, or a functional fragment thereof. In some embodiments, the RT domain or a RT is a M-MLV RT (e.g., wild-type M-MLV RT, a functional mutant, a functional variant, or a functional fragment thereof). In some embodiments, the RT domain or a RT is a M-MLV RT (e.g., a reference M-MLV RT, a functional mutant, a functional variant, or a functional fragment thereof). In some embodiments, a M-MLV RT, e.g., reference M-MLV RT, comprises an amino acid sequence as set forth in SEQ ID NO: 7597.

In some embodiments, a reference M-MLV RT is a wild-type M-MLV RT. An exemplary amino acid sequence of a reference M-MLV RT is provided in SEQ ID NO: 7599.

In some embodiments, the prime editor comprises a wild type M-MLV RT. An exemplary amino acid sequence of a wild type M-MLV RT is provided in SEQ ID NO: 7599.

(SEQ ID NO: 7599)
TLNIEDEHRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNKRVEDI
HPTVPNPYNLLSGLPPSHQWYTVLDLKDAFFCLRLHPTSQPLFAFEWRDPEMGISGQLTWTRLP
QGFKNSPTLFDEALHRDLADFRIQHPDLILLQYVDDLLLAATSELDCQQGTRALLQTLGNLGYR
ASAKKAQICQKQVKYLGYLLKEGQRWLTEARKETVMGQPTPKTPRQLREFLGTAGFCRLWIPG
FAEMAAPLYPLTKTGTLFNWGPDQQKAYQEIKQALLTAPALGLPDLTKPFELFVDEKQGYAKG
VLTQKLGPWRRPVAYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEGLQHNCLDILAEAHG
TRPDLTDQPLPDADHTWYTDGSSLLQEGQRKAGAAVTTETEVIWAKALPAGTSAQRAELIALTQ
ALKMAEGKKLNVYTDSRYAFATAHIHGEIYRRRGLLTSEGKEIKNKDEILALLKALFLPKRLSIIH
CPGHQKGHSAEARGNRMADQAARKAAITETPDTSTLLIENSSP

In some embodiments, the prime editor comprises a reference M-MLV RT. An exemplary amino acid sequence of a reference M-MLV RT is provided in SEQ ID NO: 7597.

(SEQ ID NO: 7597)
TLNIEDEYRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNKRVEDI
HPTVPNPYNLLSGLPPSHQWYTVLDLKDAFFCLRLHPTSQPLFAFEWRDPEMGISGQLTWTRLP
QGFKNSPTLFDEALHRDLADFRIQHPDLILLQYVDDLLLAATSELDCQQGTRALLQTLGNLGYR
ASAKKAQICQKQVKYLGYLLKEGQRWLTEARKETVMGQPTPKTPRQLREFLGTAGFCRLWIPG
FAEMAAPLYPLTKTGTLFNWGPDQQKAYQEIKQALLTAPALGLPDLTKPFELFVDEKQGYAKG
VLTQKLGPWRRPVAYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEGLQHNCLDILAEAHG
TRPDLTDQPLPDADHTWYTDGSSLLQEGQRKAGAAVTTETEVIWAKALPAGTSAQRAELIALTQ
ALKMAEGKKLNVYTDSRYAFATAHIHGEIYRRRGLLTSEGKEIKNKDEILALLKALFLPKRLSIIH
CPGHQKGHSAEARGNRMADQAARKAAITETPDTSTLLIENSSP

In some embodiments, the prime editor comprises a M-MMLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 7597, where X is any amino acid other than the wild type amino acid. In some embodiments, the prime editor comprises a M-MLV RT comprising one or more of amino acid substitutions P5IL, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO: 7597. In some embodiments, the prime editor comprises a M-MLV RT comprising one or more amino acid substitutions D200N, T330P, L603W, T306K, and W313F as compared to the reference M-MLV RT as set forth in SEQ ID NO: 7597. In some embodiments, the prime editor comprises a M-MLV RT comprising amino acid substitutions D200N, T330P, L603W, T306K, and W313F as compared to the reference M-MLV RT as set forth in SEQ ID NO: 7597.

In some embodiments, the prime editor comprises a M-MLV RT comprising one or more of amino acid substitutions D200N, T330P, L603W, T306K, and W313F as compared to a wild type M-MMLV RT as set forth in SEQ ID NO: 7599. In some embodiments, the prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 7597, 7599, or 7564. In some embodiments, the prime editor comprises a M-MLV RT that comprises an amino acid sequence that is selected from the group consisting of SEQ ID NOs: 7597, 7599, or 7564 or a variant or fragment thereof. In some embodiments, the prime editor comprises a M-MLV RT that comprises an amino acid sequence set forth in SEQ ID NO: 7564

(SEQ ID NO: 7564)
TLNIEDEYRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNKRVEDI
HPTVPNPYNLLSGLPPSHQWYTVLDLKDAFFCLRLHPTSQPLFAFEWRDPEMGISGQLTWTRLP
QGFKNSPTLFNEALHRDLADFRIQHPDLILLQYVDDLLLAATSELDCQQGTRALLQTLGNLGYR
ASAKKAQICQKQVKYLGYLLKEGQRWLTEARKETVMGQPTPKTPRQLREFLGKAGFCRLFIPGF
AEMAAPLYPLTKPGTLFNWGPDQQKAYQEIKQALLTAPALGLPDLTKPFELFVDEKQGYAKGV
LTQKLGPWRRPVAYLSKKLDPVAAGWPPCLRMVAAIAVLTKDAGKLTMGQPLVILAPHAVEA
LVKQPPDRWLSNARMTHYQALLLDTDRVQFGPVVALNPATLLPLPEEGLQHNCLDILAEAHGT
RPDLTDQPLPDADHTWYTDGSSLLQEGQRKAGAAVTTETEVIWAKALPAGTSAQRAELIALTQ
ALKMAEGKKLNVYTDSRYAFATAHIHGEIYRRRGWLTSEGKEIKNKDEILALLKALFLPKRLSII
HCPGHQKGHSAEARGNRMADQAARKAAITETPDTSTLLIENSSP

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a wild type RT (e.g., SEQ ID NO: 7599). In some embodiments, the RT variant comprises a fragment of a wild type RT (e.g., SEQ ID NO: 7599), such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the wild type RT (e.g., SEQ ID NO: 7599). In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding wild type RT (M-MLV reverse transcriptase) (e.g., SEQ ID NO: 7599).

In some embodiments, an RT variant may be a functional fragment of a reference RT that have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., SEQ ID NO: 7597. In some embodiments, the RT variant comprises a fragment of a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of a reference RT (e.g., SEQ ID NO: 7597). In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a reference RT (e.g., M-MLV RT) (e.g., SEQ ID NO: 7597).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the functional RT variant, e.g., a functional MMLV RT variant, is truncated at the C-terminus to abolish or reduce RNAaseH activity and still retain DNA polymerase activity.

In some embodiments, a prime editing composition or a prime editing system disclosed herein comprises a polynucleotide (e.g., a DNA, a RNA, e.g., a mRNA) that encodes a M-MLV RT. In some embodiments, the polynucleotide encodes a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 7597, 7599, or 7564. In some embodiments, the polynucleotide encodes a M-MLV RT that comprises an amino acid sequence that is selected from the group consisting of SEQ ID NOs: 7597, 7599, or 7564. In some embodiments, the polynucleotide encodes a M-MLV RT that comprises an amino acid sequence that is set forth in SEQ ID NO: 7564.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT. In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 7593, 7567, 7562, 7568, 7569, 7570, 7571-7592 [0107]. In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in SEQ ID NOs: 7593, 7567, 7562, 7568, 7569, 7570, 7571-7592.

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprises a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), Cas 4D, and homologues, modified or engineered variants, mutants, and/or functional fragments thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis. Non-limiting examples of suitable organism include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis).

In some embodiments, a Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or functional fragment of a wild type Cas protein. In some embodiments, a Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain e.g., an amino acid substitution that reduces or abolishes nuclease activity of the RuvC domain. In some embodiments, the Cas9 nickase comprises a D1OX amino acid substitution compared to a wild type S. pyogenes Cas9, wherein X is any amino acid other than D. In some embodiments, a prime editor comprises a Cas9 nickase having an amino acid substitution in the HNH domain e.g., an amino acid substitution that reduces or abolishes nuclease activity of the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some embodiments, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide, e.g., comprising an amino acid sequence as set forth in NCBI Accession No. WP_038431314 or a fragment or variant thereof. In some embodiments, a Cas9 polypeptide is a SaCas9 polypeptide, e.g., comprising an amino acid sequence as set forth in Uniprot Accession No. J7RUA5 or a fragment or variant thereof. In some embodiments, a Cas9 polypeptide is a ScCas9 polypeptide, e.g., comprising an amino acid sequence as set forth in Uniprot Accession No. A0A3P5YA78 or a fragment or variant thereof. In some embodiments, a Cas9 polypeptide is a StCas9 polypeptide, e.g., comprising an amino acid sequence as set forth in NCBI Accession No. WP_007896501.1 or a fragment or variant thereof. In some embodiments, a Cas9 polypeptide is a SluCas9 polypeptide, e.g., comprising an amino acid sequence as set forth in any of NCBI Accession No. WP_230580236.1 or WP_250638315.1 or WP_242234150.1, WP_241435384.1, WP_002460848.1, KAK58371.1, or a fragment or variant thereof. In some embodiments, a Cas9 polypeptide is a NmCas9 polypeptide, e.g., comprising an amino acid sequence as set forth in any of NCBI Accession No. WP_002238326.1 or WP_061704949.1 or a fragment or variant thereof. In some embodiments, a Cas9 polypeptide is a CjCas9 polypeptide, e.g., comprising an amino acid sequence as set forth in any of NCBI Accession No. WP_100612036.1, WP_116882154.1, WP_116560509.1, WP_116484194.1, WP_116479303.1, WP_115794652.1, WP_100624872.1, or a fragment or variant thereof. In some embodiments, a Cas9 polypeptide is a FnCas9 polypeptide, e.g., comprising the amino acid sequence as set forth in Uniprot Accession No. AOQ5Y3 or a fragment or variant thereof. In some embodiments, a Cas9 polypeptide is a TdCas9 polypeptide, e.g., comprising the amino acid sequence as set forth in NCBI Accession No. WP_147625065.1 or a fragment or variant thereof. In some embodiments, a Cas9 polypeptide is a chimera comprising domains from two or more of the organisms described herein or those known in the art. In some embodiments, a Cas9 polypeptide is a Cas9 polypeptide from Streptococcus macacae, e.g., comprising the amino acid sequence as set forth in NCBI Accession No. WP_003079701.1 or a fragment or variant thereof. In some embodiments, a Cas9 polypeptide is a Cas9 polypeptide generated by replacing a PAM interaction domain of a SpCas9 with that of a Streptococcus macacae Cas9 (Spy-mac Cas9). Exemplary Cas sequences are provided in Table 54 below.

In some embodiments, a Cas9 protein comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 7593, 7567, 7562, 7568, 7569, 7570, 7571-7592.

In some embodiments, a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 7593, 7562, 7568, 7570, 7571, 7573, 7574, 7576, 7577, 7579, 7580, 7582, 7583, 7585, 7586, 7588, 7589, 7591, and 7592 [0125]. In some embodiments, a Cas9 protein comprises an amino acid sequence that is selected from the group consisting of SEQ ID NOs: 7593, 7567, 7562, 7568, 7569, 7570, 7571-7592. In some embodiments, a prime editor comprises a Cas9 protein that comprises an amino acid sequence that lacks a N-terminus methionine relative to an amino acid sequence set forth in any one of SEQ ID NOs: 7567, 7562, 7569, 7570, 7572, 7573, 7575, 7576, 7578, 7579, 7581, 7582, 7587, 7588, 7590, 7591.

In some embodiments, the prime editing compositions or prime editing systems disclosed herein comprises a polynucleotide (e.g., a DNA, or an RNA, e.g., an mRNA) that encodes a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 7593, 7567, 7562, 7568, 7569, 7570, 7571-7592, set forth in Table 54 below.

TABLE 54
Exemplary Cas protein sequences

| SEQ | | |
|---|---|---|
| ID | Sequence | |
| NO: | description | Amino acid sequence |
| 7567 | wild type | MDKKYSIGLDIGTNSVGWAVITDEYKVPSKKFKVLGNTDRHSIKKNLIGALLFDSGE |
| | Streptococcus | TAEATRLKRTARRRYTRRKNRICYLQEIFSNEMAKVDDSFFHRLEESFLVEEDKKHE |
| | Pyogenes Cas9 | RHPIFGNIVDEVAYHEKYPTIYHLRKKLVDSTDKADLRLIYLALAHMIKFRGHFLIEG |
| | (SpCas9) | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 7562 | SpCas9 H840A | MDKKYSIGLDIGTNSVGWAVITDEYKVPSKKFKVLGNTDRHSIKKNLIGALLFDSGE |
| | nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 7568 | Met (−) SpCas9 | DKKYSIGLDIGTNSVGWAVITDEYKVPSKKFKVLGNTDRHSIKKNLIGALLFDSGET |
| | H839A nickase | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 7569 | wild type | MNQKFILGLDIGITSVGYGLIDYETKNIIDAGVRLFPEANVENNEGRRSKRGSRRLKR |
| | Staphylococcus | RRIHRLERVKKLLEDYNLLDQSQIPQSTNPYAIRVKGLSEALSKDELVIALLHIAKRR |
| | lugdunensis | GIHKIDVIDSNDDVGNELSTKEQLNKNSKLLKDKFVCQIQLERMNEGQVRGEKNRF |
| | (Slu) Cas9 | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSENSKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 7570 | SluCas9 | MNQKFILGLDIGITSVGYGLIDYETKNIIDAGVRLFPEANVENNEGRRSKRGSRRLKR |
| | N582A nickase | RRIHRLERVKKLLEDYNLLDQSQIPQSTNPYAIRVKGLSEALSKDELVIALLHIAKRR |
| | | GIHKIDVIDSNDDVGNELSTKEQLNKNSKLLKDKFVCQIQLERMNEGQVRGEKNRF |
| | | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSEASKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 7571 | Met (−) | NQKFILGLDIGITSVGYGLIDYETKNIIDAGVRLFPEANVENNEGRRSKRGSRRLKRR |
| | SluCas9 | RIHRLERVKKLLEDYNLLDQSQIPQSTNPYAIRVKGLSEALSKDELVIALLHIAKRRGI |
| | nickase | HKIDVIDSNDDVGNELSTKEQLNKNSKLLKDKFVCQIQLERMNEGQVRGEKNRFKT |
| | | ADIIKEIIQLLNVQKNFHQLDENFINKYIELVEMRREYFEGPGKGSPYGWEGDPKAW |
| | | YETLMGHCTYFPDELRSVKYAYSADLFNALNDLNNLVIQRDGLSKLEYHEKYHIIE |
| | | NVFKQKKKPTLKQIANEINVNPEDIKGYRITKSGKPQFTEFKLYHDLKSVLFDQSILE |
| | | NEDVLDQIAEILTIYQDKDSIKSKLTELDILLNEEDKENIAQLTGYTGTHRLSLKCIRL |
| | | VLEEQWYSSRNQMEIFTHLNIKPKKINLTAANKIPKAMIDEFILSPVVKRTFGQAINLI |
| | | NKIIEKYGVPEDIIIELARENNSKDKQKFINEMQKKNENTRKRINEIIGKYGNQNAKR |
| | | LVEKIRLHDEQEGKCLYSLESIPLEDLLNNPNHYEVDHIIPRSVSFDNSYHNKVLVKQ |
| | | SEASKKSNLTPYQYFNSGKSKLSYNQFKQHILNLSKSQDRISKKKKEYLLEERDINKF |
| | | EVQKEFINRNLVDTRYATRELTNYLKAYFSANNMNVKVKTINGSFTDYLRKVWKF |
| | | KKERNHGYKHHAEDALIIANADFLFKENKKLKAVNSVLEKPEIESKQLDIQVDSEDN |
| | | YSEMFIIPKQVQDIKDFRNFKYSHRVDKKPNRQLINDTLYSTRKKDNSTYIVQTIKDI |
| | | YAKDNTTLKKQFDKSPEKFLMYQHDPRTFEKLEVIMKQYANEKNPLAKYHEETGE |
| | | YLTKYSKKNNGPIVKSLKYIGNKLGSHLDVTHQFKSSTKKLVKLSIKPYRFDVYLTD |
| | | KGYKFITISYLDVLKKDNYYYIPEQKYDKLKLGKAIDKNAKFIASFYKNDLIKLDGEI |
| | | YKIIGVNSDTRNMIELDLPDIRYKEYCELNNIKGEPRIKKTIGKKVNSIEKLTTDVLGN |
| | | VFTNTQYTKPQLLFKRGN |
| 7572 | Staphylococcus | MKRNYILGLDIGITSVGYGIIDYETRDVIDAGVRLFKEANVENNEGRRSKRGARRLK |
| | aureus Cas9 | RRRRHRIQRVKKLLFDYNLLTDHSELSGINPYEARVKGLSQKLSEEEFSAALLHLAK |
| | (SaCas9) | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEENSKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 7573 | SaCas9 N580A | MKRNYILGLDIGITSVGYGIIDYETRDVIDAGVRLFKEANVENNEGRRSKRGARRLK |
| | nickase | RRRRHRIQRVKKLLFDYNLLTDHSELSGINPYEARVKGLSQKLSEEEFSAALLHLAK |
| | | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEEASKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 7574 | Met (−) SaCas9 | KRNYILGLDIGITSVGYGIIDYETRDVIDAGVRLFKEANVENNEGRRSKRGARRLKR |
| | nickase | RRRHRIQRVKKLLFDYNLLTDHSELSGINPYEARVKGLSQKLSEEEFSAALLHLAKR |
| | | RGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEI |
| | | IENAELLDQIAKILTIYQSSEDIQEELTNLNSELTQEEIEQISNLKGYTGTHNLSLKAIN |
| | | LILDELWHTNDNQIAIFNRLKLVPKKVDLSQQKEIPTTLVDDFILSPVVKRSFIQSIKVI |
| | | NAIIKKYGLPNDIIIELAREKNSKDAQKMINEMQKRNRQTNERIEEIIRTTGKENAKY |
| | | LIEKIKLHDMQEGKCLYSLEAIPLEDLLNNPFNYEVDHIIPRSVSFDNSFNNKVLVKQ |
| | | EEASKKGNRTPFQYLSSSDSKISYETFKKHILNLAKGKGRISKTKKEYLLEERDINRFS |
| | | VQKDFINRNLVDTRYATRGLMNLLRSYFRVNNLDVKVKSINGGFTSFLRRKWKFK |
| | | KERNKGYKHHAEDALIIANADFIFKEWKKLDKAKKVMENQMFEEKQAESMPEIETE |
| | | QEYKEIFITPHQIKHIKDFKDYKYSHRVDKKPNRELINDTLYSTRKDDKGNTLIVNNL |
| | | NGLYDKDNDKLKKLINKSPEKLLMYHHDPQTYQKLKLIMEQYGDEKNPLYKYYEE |
| | | TGNYLTKYSKKDNGPVIKKIKYYGNKLNAHLDITDDYPNSRNKVVKLSLKPYRFDV |
| | | YLDNGVYKFVTVKNLDVIKKENYYEVNSKCYEEAKKLKKISNQAEFIASFYNNDLI |
| | | KINGELYRVIGVNNDLLNRIEVNMIDITYREYLENMNDKRPPRIIKTIASKTQSIKKYS |
| | | TDILGNLYEVKSKKHPQIIKKG |
| 7575 | SpCas9-NG | MDKKYSIGLDIGTNSVGWAVITDEYKVPSKKFKVLGNTDRHSIKKNLIGALLFDSGE |
| | (VRVRFRR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 7576 | spCas9-NG | MDKKYSIGLDIGTNSVGWAVITDEYKVPSKKFKVLGNTDRHSIKKNLIGALLFDSGE |
| | (H840A_VRVRFRR) | TAEATRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 7577 | Met (−) | DKKYSIGLDIGTNSVGWAVITDEYKVPSKKFKVLGNTDRHSIKKNLIGALLFDSGET |
| | SpCas9-NG | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 7578 | spCas9-NGA | MDKKYSIGLDIGTNSVGWAVITDEYKVPSKKFKVLGNTDRHSIKKNLIGALLFDSGE |
| | (VRQR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 7579 | spCas9-NGA | MDKKYSIGLDIGTNSVGWAVITDEYKVPSKKFKVLGNTDRHSIKKNLIGALLFDSGE |
| | (H840A_VRQR) | TAEATRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 7580 | Met(−) spCas9- | DKKYSIGLDIGTNSVGWAVITDEYKVPSKKFKVLGNTDRHSIKKNLIGALLFDSGET |
| | NGA Nickase | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 7581 | SpRY Cas9 | MDKKYSIGLDIGTNSVGWAVITDEYKVPSKKFKVLGNTDRHSIKKNLIGALLFDSGE |
| | | TAERTRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 7582 | SpRY Cas9 | MDKKYSIGLDIGTNSVGWAVITDEYKVPSKKFKVLGNTDRHSIKKNLIGALLFDSGE |
| | (H840A) | TAERTRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 7583 | Met(−) SpRY | DKKYSIGLDIGTNSVGWAVITDEYKVPSKKFKVLGNTDRHSIKKNLIGALLFDSGET |
| | Cas9 Nickase | AERTRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 7584 | sRGN3.1 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 7585 | SRGN3.1 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 7586 | Met (−) | NQKFILGLDIGITSVGYGLIDYETKNIIDAGVRLFPEANVENNEGRRSKRGSRRLKRR |
| | sRGN3.1 | RIHRLERVKLLLTEYDLINKEQIPTSNNPYQIRVKGLSEILSKDELAIALLHLAKRRGI |
| | (N584A) | HNVDVAADKEETASDSLSTKDQINKNAKFLESRYVCELQKERLENEGHVRGVENRF |
| | Nickase | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTNYLKAYFSANNMNVKVKTINGSFTDYLRK |
| | | VWKFKKERNHGYKHHAEDALIIANADFLFKENKKLKAVNSVLEKPEIETKQLDIQV |
| | | DSEDNYSEMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEKNPLAKYH |
| | | EETGEYLTKYSKKNNGPIVKSLKYIGNKLGSHLDVTHQFKSSTKKLVKLSIKNYRFD |
| | | VYLTEKGYKFVTIAYLNVFKKDNYYYIPKDKYQELKEKKKIKDTDQFIASFYKNDLI |
| | | KLNGDLYKIIGVNSDDRNIIELDYYDIKYKDYCEINNIKGEPRIKKTIGKKTESIEKFTT |
| | | DVLGNLYLHSTEKAPQLIFKRGL |
| 7587 | sRGN3.3 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 7588 | sRGN3.3 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 7589 | Met (−) | NQKFILGLDIGITSVGYGLIDYETKNIIDAGVRLFPEANVENNEGRRSKRGSRRLKRR |
| | sRGN3.3 | RIHRLERVKLLLTEYDLINKEQIPTSNNPYQIRVKGLSEILSKDELAIALLHLAKRRGI |
| | (N584A) | HNVDVAADKEETASDSLSTKDQINKNAKFLESRYVCELQKERLENEGHVRGVENRF |
| | Nickase | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTSYLKAYFSANNMDVKVKTINGSFTNHLRK |
| | | VWRFDKYRNHGYKHHAEDALIIANADFLFKENKKLQNTNKILEKPTIENNTKKVTV |
| | | EKEEDYNNVFETPKLVEDIKQYRDYKFSHRVDKKPNRQLINDTLYSTRMKDEHDYI |
| | | VQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 7590 | SpG | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 7591 | SpG(H840A) | MDKKYSIGLDIGTNSVGWAVITDEYKVPSKKFKVLGNTDRHSIKKNLIGALLFDSGE |
| | Nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 7592 | Met (−) | DKKYSIGLDIGTNSVGWAVITDEYKVPSKKFKVLGNTDRHSIKKNLIGALLFDSGET |
| | SpG(H839A) | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprises a mutation at amino acid D10 as compared to a wild type SpCas9 as set forth in SEQ ID NO: 7567, or a corresponding mutation thereof. In some embodiments, the Cas9 comprises a D10A mutation as compared to a wild type SpCas9 as set forth in SEQ ID NO: 7567, or a corresponding mutation thereof. In some embodiments, the Cas9 polypeptide comprises a mutation at amino acid D10, G12, and/or G17 as compared to a wild type SpCas9 as set forth in SEQ ID NO: 7567, or a corresponding mutation thereof. In some embodiments, the Cas9 polypeptide comprises a D10A mutation, a G12A mutation, and/or a G17A mutation as compared to a wild type SpCas9 as set forth in SEQ ID NO: 7567, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprises a mutation at amino acid H840 as compared to a wild type SpCas9 as set forth in SEQ ID NO: 7567, or a corresponding mutation thereof. In some embodiments, the Cas9 polypeptide comprises a H840A mutation as compared to a wild type SpCas9 as set forth in SEQ ID NO: 7567, or a corresponding mutation thereof. In some embodiments, the Cas9 polypeptide comprises a mutation at amino acid E762, D839, H840, N854, N856, N863, H982, H983, A984, D986, and/or a A987 as compared to a wild type SpCas9 as set forth in SEQ ID NO: 7567, or a corresponding mutation thereof. In some embodiments, the Cas9 polypeptide comprises a E762A, D839A, H840A, N854A, N856A, N863A, H982A, H983A, A984A, and/or a D986A mutation as compared to a wild type SpCas9 as set forth in SEQ ID NO: 7567, or a corresponding mutation thereof. In some embodiments, the Cas9 polypeptide comprises a mutation at amino acid residue R221, N394, and/or H840 as compared to a wild type SpCas9 (e.g., SEQ ID NO: 7567). In some embodiments, the Cas9 polypeptide comprises a R221K, N394L, and/or H840A mutation as compared to a wild type SpCas9 as set forth in SEQ ID NO: 7567, or a corresponding mutation thereof. In some embodiments, the Cas9 polypeptide comprises a mutation at amino acid residue R220, N393, and/or H839 as compared to a wild type SpCas9 (e.g., SEQ ID NO: 7567) lacking a N-terminal methionine, or a corresponding mutation thereof. In some embodiments, the Cas9 polypeptide comprises a R220K, N393K, and/or H839A mutation as compared to a wild type SpCas9 (as set forth in SEQ ID NO: 7567, lacking a N-terminal methionine, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 7567 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 7567, or corresponding mutations thereof.

In some embodiments, the N-terminal methionine is removed from the amino acid sequence of a Cas9 nickase, or from any Cas9 variant, ortholog, or equivalent disclosed or contemplated herein. For example, methionine-minus (Met (-)) Cas9 nickases include any one of the sequences set forth in SEQ ID NOs: 7568, 7571, 7574, 7577, 7580, 7583, 7586, 7589, 7592 or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70%, at least about 80%, at least about 90%, at least about 95%, at least about 96%, at least about 97%, at least about 98%, at least about 99%, at least about 99.5%, or at least about 99.9% sequence identity to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 49 below. It should be appreciated that for each of the variants provided, the Cas protein comprises one or more of the amino acid substitutions as indicated compared to a wild type Cas protein sequence, for example, the Cas9 as set forth in SEQ ID NO: 7567. The PAM motifs as shown in Table 49 below are in the order of 5′ to 3′. In some embodiments, the Cas proteins of the disclosure can also be used to direct transcriptional control of target sequences, for example silencing transcription by sequence-specific binding to target sequences. In some embodiments, a Cas protein described herein may have one or mutations in a PAM recognition motif. In some embodiments, a Cas protein described herein may have altered PAM specificity.

As used in PAM sequences in Table 49, “N” refers to any one of nucleotides A, G, C, and T, “R” refers to nucleotide A or G, and “Y”, refers to nucleotide C or T.

TABLE 49
Cas protein variants and corresponding PAM sequences

| Variant | PAM |
|---|---|
| spCas9 (wild type) | NGG, NGA, NAG, NGNGA |
| spCas9-VRVRFRR | NG |
| R1335V/L1111R/D1135V/G1218R/ | |
| E1219F/A1322R/T1337R | |
| spCas9-VQR (D1135V/R1335Q/ | NGA |
| T1337R) | |
| spCas9-EQR | NGA |
| (D1135E/R1335Q/T1337R) | |
| spCas9-VRER | NGCG |
| (D1135V/G1218R/R1335E/T1337R) | |
| spCas9-VRQR | NGA |
| (D1135V, G1218R, R1335Q, T1337R) | |
| Cas9-NG | NGN |
| (L1111R, D1135V, G1218R, E1219F, | |
| A1322R, T1337R, R1335V) | |
| SpG Cas9 | NGN |
| (D1135L, S1136W, G1218K, E1219Q, | |
| R1335Q, T1337R) | |
| SyRY Cas9 | NRN |
| (A61R, L1111R, N1317R, A1322R, | |
| and R1333P) | |
| xCas9 (E480K, E543D, E1219V, | NGN |
| K294R, Q1256K, A262T, S409I, M694I) | |
| SluCa9 | NNGG |
| sRGN1, sRGN2, sRGN4, sRGN3.1, | NNGG |
| sRGN3.3 | |
| saCas9 | NNGRRT, NNGRRN |
| saCas9-KKH | NNNRRT |
| (E782K, N968K, R1015H) | |
| spCas9-MQKSER | NGCG/NGCN |
| (D1135M, S1136Q, G1218K, E1219S, | |
| R1335E, T1337R) | |
| spCas9-LRKIQK | NGTN |
| (D1135L, S1136R, G1218K, E1219I, | |
| R1335Q, T1337K) | |
| spCas9-LRVSQK | NGTN |
| (D1135L, S1136R, G1218V, E1219S, | |
| R1335Q, T1337K) | |
| spCas9-LRVSQL | NGTN |
| (D1135L, S1136R, G12118V, E1219S, | |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A6iR, LiiiR, Di1i35V, R22iK, A262T, R324L, N394K, S4091, S4091, E427G, E480K, M495V, N497A, Y5i5N, K526E, F539S, E543D, R654L, R66iA, R66iL, R69iA, N692A, M694A, M6941, Q695A, H698A, R753G, M7631, K848A, K890N, Q926A, Ki003A, RiO60A, LiiiiR, Riii4G, Dii35E, Dii35L, Dii35N, Sii36W, Vii39A, Dii80G, Gi2i8K, G1218R, G1218S, E1219Q, E1219V, E1219V, Q1221H, P1249S, E1253K, N1317R, A1320V, P1321S, A1322R, 11322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 7567.

In some embodiments, a prime editor comprises a SaCas9 polypeptide. In some embodiments, the SaCas9 polypeptide comprises one or more of mutations E782K, N968K, and RiO15H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G368D, I369K, H371L, T375S, T376G, and T1227K as compared to the wild type ScCas9. In some embodiments, a prime editor comprises a St1 Cas9 polypeptide, a St3 Cas9 polypeptide, or a SluCas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 7567):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc.).

In some embodiments, a circular permutant Cas9 comprises any one of the following structures (amino acid positions as set forth in SEQ ID NO: 7567-1368 amino acids of UniProtKB-Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc.).

In some embodiments, a circular permutant Cas9 comprises any one of the following structures (amino acid positions as set forth in SEQ ID NO: 7567-1368 amino acids of UniProtKB-Q99ZW2

N-terminus-[103-1368]-[optional linker]-[1-102]-C-terminus:

N-terminus-[1029-1368]-[optional linker]-[1-1028]-C-terminus;

N-terminus-[1042-1368]-[optional linker]-[1-1041]-C-terminus;

N-terminus-[1250-1368]-[optional linker]-[1-1249]-C-terminus; or

N-terminus-[1301-1368]-[optional linker]-[1-1300]-C-terminus, or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the 95% or more of the C-terminal amino acids of a Cas9 (e.g., amino acids about 1300-1368 as set forth in SEQ ID No: 7567 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more amino acids of the C-terminal of a Cas9 (e.g., Cas9 of SEQ ID NO: 7567 or corresponding amino acid positions thereof). The N-terminal portion may correspond to 95% or more of the amino acids of the N-terminal of a Cas9 (e.g., amino acids about 1-1300 as set forth in SEQ ID No: 7567 or a ortholog or a variant thereof), or 90%, 85%, 80%, 75%, 70%, 65%, 60%, 55%, 50%, 45%, 40%, 35%, 30%, 25%, 20%, 15%, 10%, or 5% or more of the N-terminal amino acids of a Cas9 (e.g., as set forth in SEQ ID NO: 7567 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID No: 7567 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID No: 7567 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID No: 7567 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g. as set forth in SEQ ID No: 7567 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID No: 7567 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 7567: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID No: 7567 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²70, Cas9-CP³¹⁰, Cas9-CP¹⁰¹⁰, Cas9-CP¹⁰¹⁶, Cas9-CPi⁰²³, Cas9-CPi⁰²⁹, Cas9-CP¹⁰⁴¹, Cas9-CP¹²⁴7, Cas9-CP¹²⁴⁹, and Cas9-CP¹²⁸², respectively. This description is not meant to be limited to making CP variants from SEQ ID NO: 18, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 18). In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Casl3b, a Casl3c, a Casl3d, a Cas14, a Csn2, an xCas9, an SpCas9-NG, a circularly permuted Cas9, or an Argonaute (Ago) domain, or a functional variant or fragment thereof.

### Exemplary Cas Proteins and Nomenclature are Shown in Table 50 Below:

TABLE 50
Exemplary Cas proteins and nomenclature

| | |
|---|---|
| Legacy nomenclature | Current nomenclature |

type II CRISPR-Cas enzyme

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, prime editors described herein may also comprise Cas proteins other than Cas9. For example, in some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a Cas4 polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12e, Cas12d, Cas13, Cas14a, Cas14b, Cas14c, Cas14d, Cas14e, Cas14f, Cas14g, Cas14h, Cas14u, or Cas qP protein. In some embodiments, the Cas protein is a Cas12e, Cas12d, Cas13, or Cas qP nickase.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor may further comprise more than 4, 5, 6, 7, 8, 9 or 10 NLSs.

In addition, the NLSs may be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 7548), KRTADGSEFESPKKKRKV (SEQ ID NO: 7600), KRTADGSEFEPKKKRKV (SEQ ID NO: 7555), SKRPAAIKKAGQAKKKK (SEQ ID NO: 7556), RQRRNELKRSF (SEQ ID NO: 7557), or NQSSNFGPMKGGNFGGRSSGPYGGGGQYFAKPRNQGGY (SEQ ID NO: 7558).

In some embodiments, a NLS is a monopartite NLS. For example, in some embodiments, a NLS is a SV40 large T antigen NLS (PKKKRKV). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the sequence (KRXXXXXXXXXXKKKL (Xenopus nucleoplasmin NLS) (SEQ ID NO: 7559), wherein X is any amino acid. In some embodiments, the NLS comprises a nucleoplasmin NLS sequence KRPAATKKAGQAKKKK (SEQ ID NO: 7601). In some embodiments, a NLS is a noncanonical sequences such as M9 of the hnRNP A1 protein, the influenza virus nucleoprotein NLS, and the yeast Gal4 protein NLS. In some embodiments, a NLS is a noncanonical sequences such as M9 of the hnRNP A1 protein, the influenza virus nucleoprotein NLS, and the yeast Gal4 protein NLS.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues. Non-limiting examples of NLS sequences are provided in Table 51 below.

Other non-limiting examples of NLS sequences are provided in Table 51 below.

TABLE 51
Exemplary nuclear localization sequences

| Description | Sequence |
|---|---|
| NLS of SV40 Large T-AG | PKKKRKV (SEQ ID NO: 7619) |
| NLS | MKRTADGSEFESPKKKRKV (SEQ ID NO: 7547) |
| NLS | MDSLLMNRRKFLYQFKNVRWAKGRRETYLC (SEQ ID NO: |
| | 7548) |
| NLS of Nucleoplasmin | AVKRPAATKKAGQAKKKKLD (SEQ ID NO: 7549) |
| NLS of EGL-13 | MSRRRKANPTKLSENAKKLAKEVEN (SEQ ID NO: 7550) |
| NLS of C-Myc | PAAKRVKLD (SEQ ID NO: 7620) |
| NLS of Tus-protein | KLKIKRPVK (SEQ ID NO: 7621) |
| NLS of polyoma large T-AG | VSRKRPRP (SEQ ID NO: 7622) |
| NLS of Rev protein | RQARRNRRRRWRERNR (SEQ ID NO: 7551) |
| NLS of Hepatitis D virus antigen | EGAPPAKRAR (SEQ ID NO: 7552) |
| NLS of murine p53 | PPQPKKKPLDGE (SEQ ID NO: 7553) |
| C terminal linker and NLS of an | SGGSKRTADGSEFEPKKKRKV (SEQ ID NO: 7554) |
| exemplary prime editor fusion | |
| protein | |
| C Terminal NLS of an | KRTADGSEFEPKKKRKV (SEQ ID NO: 7555) |
| exemplary prime editor fusion | |
| protein | |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a-trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C. essentially in same way as a contiguous intein does. In some embodiments, a split-intein is derived from a eukaryotic intein, a bacterial intein, or an archaeal intein. Preferably, the split intein so derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last $-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex-A DNA binding domain (DBD) fusions, GAL4 DNA binding domain fusions, and herpes simplex virus (HSV) BP16 protein fusions.

In some embodiments, a prime editor comprises a protein domain that is capable of modifying the intracellular half-life of the prime editor.

In some embodiments, a prime editing complex comprises a fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 7560. Sequence of an exemplary prime editor fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)] and its components are shown in Table 52.

In some embodiments, a prime editing complex comprises a fusion protein comprising a DNA binding domain (e.g., Cas9((R221K N394K H840A)) and a reverse transcriptase (e.g., a variant MMLV RT) having the following structure: [NLS]-[Cas9((R221K N394K H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 7560. Sequence of an exemplary prime editor fusion protein comprising a DNA binding domain (e.g., Cas9(H840A)) and a reverse transcriptase (e.g., a variant MMLV RT) having the following structure: [NLS]-[Cas9 (R221K N394K H840A)]-[linker]-[MMLV_RT(D200N)(T330P)(L603W)(T306K)(W313F)] and its components are shown in Table 53.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 7602). In some embodiments, the amino acid sequence of the MCP is:

(SEQ ID NO: 7603)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIAANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 7625), (G)n (SEQ ID NO: 7613), (EAAAK)n (SEQ ID NO: 7614), (GGS)n (SEQ ID NO: 7615), (SGGS)n (SEQ IDNO: 7616), (XP)n(SEQ ID NO: 7617) or any combination thereof, wherein n is independently an integer between 1 and 30, and wherein X is any amino acid. In some embodiments, the linker comprises the amino acid sequence (GGS)n (SEQ ID NO: 7618), wherein n is 1, 3, or 7. In some embodiments, the linker comprises the amino acid sequence SGSETPGTSESATPES (SEQ ID NO: 7604). In some embodiments, the linker comprises the amino acid sequence SGGSSGGSSGSETPGTSESATPESSGGSSGGS (SEQ ID NO: 7609). In some embodiments, the linker comprises the amino acid sequence SGGSGGSGGS (SEQ ID NO: 7605). In some embodiments, the linker comprises the amino acid sequence SGGS (SEQ ID NO: 7610). In other embodiments, the linker comprises the amino acid sequence SGGSSGGSSGSETPGTSESATPESAGSYPYDVPDYAGSAAPAAKKKKLDGSGSGGSSGGS (SEQ ID NO: 7606).

In some embodiments, a linker comprises 1-100 amino acids. In some embodiments, the linker comprises the amino acid sequence SGSETPGTSESATPES (SEQ ID NO: 7604). In some embodiments, the linker comprises the amino acid sequence SGGSSGGSSGSETPGTSESATPESSGGSSGGS (SEQ ID NO: 7609). In some embodiments, the linker comprises the amino acid sequence SGGSGGSGGS (SEQ ID NO: 7605). In some embodiments, the linker comprises the amino acid sequence SGGS (SEQ ID NO: 7610). In some embodiments, the linker comprises the amino acid sequence GGSGGS (SEQ ID NO: 7611), GGSGGSGGS (SEQ ID NO: 7612), SGGSSGGSSGSETPGTSESATPESAGSYPYDVPDYAGSAAPAAKKKKLDGSGSGGSSGGS (SEQ ID NO: 7606), or SGGSSGGSSGSETPGTSESATPESSGGSSGGSS (SEQ ID NO: 7563).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell. In some embodiments, an exemplary protein described herein may lack a methionine residue at the N-terminus.

In some embodiments, a prime editor fusion protein comprises a Cas9(H840A) nickase and a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT. The amino acid sequence of an exemplary prime editor fusion protein and its individual components in shown in Table 52. In some embodiments, a prime editor fusion protein comprises a Cas9 (R221K N394K H840A) nickase and a M-MLV RT that comprises amino acid substitutions D200N, T330P, T306K, W313F, and L603W compared to a wild type M-MLV RT. The amino acid sequence of an exemplary Prime editor fusion protein and its individual components in shown in Table 53. In some embodiments an exemplary prime editor protein may comprise an amino acid sequence as set forth in any of the SEQ ID NOs: 7560, 7561, 7565 or 7566.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any one of the exemplary prime editor fusion proteins provided herein, or any of the prime editor fusion sequences described herein or known in the art.

TABLE 52
lists an exemplary prime editor fusion protein and its individual components

| DESCRIPTION | SEQUENCE |
|---|---|
| Exemplary Prime | MKRTADGSEFESPKKKRKVDKKYSIGLDIGTNSVGWAVITDEYKVPSKKFK |
| Editor [NLS]- | VLGNTDRHSIKKNLIGALLFDSGETAEATRLKRTARRRYTRRKNRICYLQ |
| [Cas9(H840A)]- | EIFSNEMAKVDDSFFHRLEESFLVEEDKKHERHPIFGNIVDEVAYHEKYPT |
| [linker]- | IYHLRKKLVDSTDKADLRLIYLALAHMIKFRGHFLIEGDLNPDNSDVDKL |
| [MMLV_RT(D200N) | FIQLVQTYNQLFEENPINASGVDAKAILSARLSKSRRLENLIAQLPGEKKN |
| (T330P)(L603W) | GLFGNLIALSLGLTPNFKSNFDLAEDAKLQLSKDTYDDDLDNLLAQIGDQ |
| (T306K)(W313F)]- | YADLFLAAKNLSDAILLSDILRVNTEITKAPLSASMIKRYDEHHQDLTLLK |
| [NLS] (SEQ | ALVRQQLPEKYKEIFFDQSKNGYAGYIDGGASQEEFYKFIKPILEKMDGT |
| ID NO: 7560) | EELLVKLNREDLLRKQRTFDNGSIPHQIHLGELHAILRRQEDFYPFLKDN |
| | REKIEKILTFRIPYYVGPLARGNSRFAWMTRKSEETITPWNFEEVVDKGAS |
| | AQSFIERMTNFDKNLPNEKVLPKHSLLYEYFTVYNELTKVKYVTEGMRK |
| | PAFLSGEQKKAIVDLLFKTNRKVTVKQLKEDYFKKIECFDSVEISGVEDR |
| | FNASLGTYHDLLKIIKDKDFLDNEENEDILEDIVLTLTLFEDREMIEERLKT |
| | YAHLFDDKVMKQLKRRRYTGWGRLSRKLINGIRDKQSGKTILDFLKSDG |
| | FANRNFMQLIHDDSLTFKEDIQKAQVSGQGDSLHEHIANLAGSPAIKKGIL |
| | QTVKVVDELVKVMGRHKPENIVIEMARENQTTQKGQKNSRERMKRIEE |
| | GIKELGSQILKEHPVENTQLQNEKLYLYYLQNGRDMYVDQELDINRLSDY |
| | DVDAIVPQSFLKDDSIDNKVLTRSDKNRGKSDNVPSEEVVKKMKNYWRQ |
| | LLNAKLITQRKFDNLTKAERGGLSELDKAGFIKRQLVETRQITKHVAQIL |
| | DSRMNTKYDENDKLIREVKVITLKSKLVSDFRKDFQFYKVREINNYHHAH |
| | DAYLNAVVGTALIKKYPKLESEFVYGDYKVYDVRKMIAKSEQEIGKATAK |
| | YFFYSNIMNFFKTEITLANGEIRKRPLIETNGETGEIVWDKGRDFATVRKV |
| | LSMPQVNIVKKTEVQTGGFSKESILPKRNSDKLIARKKDWDPKKYGGFD |
| | SPTVAYSVLVVAKVEKGKSKKLKSVKELLGITIMERSSFEKNPIDFLEAKG |
| | YKEVKKDLIIKLPKYSLFELENGRKRMLASAGELQKGNELALPSKYVNFL |
| | YLASHYEKLKGSPEDNEQKQLFVEQHKHYLDEIIEQISEFSKRVILADANL |
| | DKVLSAYNKHRDKPIREQAENIIHLFTLTNLGAPAAFKYFDTTIDRKRYTS |
| | TKEVLDATLIHQSITGLYETRIDLSQLGGDSGGSSGGSSGSETPGTSESATP |
| | ESSGGSSGGSSTLNIEDEYRLHETSKEPDVSLGSTWLSDFPQAWAETGGMGLAVR |
| | QAPLIIPLKATSTPVSIKQYPMSQEARLGIKPHIQRLLDQGILVPCQSPWNTPLLPVK |
| | KPGTNDYRPVQDLREVNKRVEDIHPTVPNPYNLLSGLPPSHQWYTVLDLKDAFFC |
| | LRLHPTSQPLFAFEWRDPEMGISGQLTWTRLPQGFKNSPTLFNEALHRDLADFRI |
| | QHPDLILLQYVDDLLLAATSELDCQQGTRALLQTLGNLGYRASAKKAQICQKQVKY |
| | LGYLLKEGQRWLTEARKETVMGQPTPKTPRQLREFLGKAGFCRLFIPGFAEMAAP |
| | LYPLTKPGTLFNWGPDQQKAYQEIKQALLTAPALGLPDLTKPFELFVDEKQGYAK |
| | GVLTQKLGPWRRPVAYLSKKLDPVAAGWPPCLRMVAAIAVLTKDAGKLTMGQPLV |
| | ILAPHAVEALVKQPPDRWLSNARMTHYQALLLDTDRVQFGPVVALNPATLLPLPEE |
| | GLQHNCLDILAEAHGTRPDLTDQPLPDADHTWYTDGSSLLQEGQRKAGAAVTTET |
| | EVIWAKALPAGTSAQRAELIALTQALKMAEGKKLNVYTDSRYAFATAHIHGEIYRRRG |
| | WLTSEGKEIKNKDEILALLKALFLPKRLSIIHCPGHQKGHSAEARGNRMADQAARK |
| | AAITETPDTSTLLIENSSPSGGSKRTADGSEFEPKKKRKV |
| | KEY: |
| | NUCLEAR LOCALIZATION SEQUENCE (NLS) |
| | CAS9(H840A) |
| | 33-AMINO ACID LINKER |
| | M-MLV REVERSE TRANSCRIPTASE |
| Exemplary Prime | KRTADGSEFESPKKKRKVDKKYSIGLDIGTNSVGWAVITDEYKVPSKKFKVLGNTDRHSIKKNL |
| Editor [NLS]- | IGALLFDSGETAEATRLKRTARRRYTRRKNRICYLQEIFSNEMAKVDDSFFHRLEESFLVEEDKK |
| [Cas9(H840A)]- | HERHPIFGNIVDEVAYHEKYPTIYHLRKKLVDSTDKADLRLIYLALAHMIKFRGHFLIEGDLNPD |
| [linker]- | NSDVDKLFIQLVQTYNQLFEENPINASGVDAKAILSARLSKSRRLENLIAQLPGEKKNGLFGNLIA |
| [MMLV_RT(D200N) | LSLGLTPNFKSNFDLAEDAKLQLSKDTYDDDLDNLLAQIGDQYADLFLAAKNLSDAILLSDILRV |
| (T330P)(L603W) | NTEITKAPLSASMIKRYDEHHQDLTLLKALVRQQLPEKYKEIFFDQSKNGYAGYIDGGASQEEF |
| (T306K)(W313F)]- | YKFIKPILEKMDGTEELLVKLNREDLLRKQRTFDNGSIPHQIHLGELHAILRRQEDFYPFLKDNRE |
| [NLS] | KIEKILTFRIPYYVGPLARGNSRFAWMTRKSEETITPWNFEEVVDKGASAQSFIERMTNFDKNLP |
| without N- | NEKVLPKHSLLYEYFTVYNELTKVKYVTEGMRKPAFLSGEQKKAIVDLLFKTNRKVTVKQLKE |
| terminal | DYFKKIECFDSVEISGVEDRFNASLGTYHDLLKIIKDKDFLDNEENEDILEDIVLTLTLFEDREMIE |
| methionine | ERLKTYAHLFDDKVMKQLKRRRYTGWGRLSRKLINGIRDKQSGKTILDFLKSDGFANRNFMQL |
| (SEQ ID NO: | IHDDSLTFKEDIQKAQVSGQGDSLHEHIANLAGSPAIKKGILQTVKVVDELVKVMGRHKPENIVI |
| 7561) | EMARENQTTQKGQKNSRERMKRIEEGIKELGSQILKEHPVENTQLQNEKLYLYYLQNGRDMYV |
| | DQELDINRLSDYDVDAIVPQSFLKDDSIDNKVLTRSDKNRGKSDNVPSEEVVKKMKNYWRQLL |
| | NAKLITQRKFDNLTKAERGGLSELDKAGFIKRQLVETRQITKHVAQILDSRMNTKYDENDKLIR |
| | EVKVITLKSKLVSDFRKDFQFYKVREINNYHHAHDAYLNAVVGTALIKKYPKLESEFVYGDYK |
| | VYDVRKMIAKSEQEIGKATAKYFFYSNIMNFFKTEITLANGEIRKRPLIETNGETGEIVWDKGRD |
| | FATVRKVLSMPQVNIVKKTEVQTGGFSKESILPKRNSDKLIARKKDWDPKKYGGFDSPTVAYSV |
| | LVVAKVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDEIIEQI |
| | SEFSKRVILADANLDKVLSAYNKHRDKPIREQAENIIHLFTLTNLGAPAAFKYFDTTIDRKRYTST |
| | KEVLDATLIHQSITGLYETRIDLSQLGGDSGGSSGGSSGSETPGTSESATPESSGGSSGGSSTLNIE |
| | DEYRLHETSKEPDVSLGSTWLSDFPQAWAETGGMGLAVRQAPLIIPLKATSTPVSIKQYPMSQE |
| | ARLGIKPHIQRLLDQGILVPCQSPWNTPLLPVKKPGTNDYRPVQDLREVNKRVEDIHPTVPNPYN |
| | LLSGLPPSHQWYTVLDLKDAFFCLRLHPTSQPLFAFEWRDPEMGISGQLTWTRLPQGFKNSPTLF |
| | NEALHRDLADFRIQHPDLILLQYVDDLLLAATSELDCQQGTRALLQTLGNLGYRASAKKAQICQ |
| | KQVKYLGYLLKEGQRWLTEARKETVMGQPTPKTPRQLREFLGKAGFCRLFIPGFAEMAAPLYP |
| | LTKPGTLFNWGPDQQKAYQEIKQALLTAPALGLPDLTKPFELFVDEKQGYAKGVLTQKLGPWR |
| | RPVAYLSKKLDPVAAGWPPCLRMVAAIAVLTKDAGKLTMGQPLVILAPHAVEALVKQPPDRW |
| | LSNARMTHYQALLLDTDRVQFGPVVALNPATLLPLPEEGLQHNCLDILAEAHGTRPDLTDQPLP |
| | DADHTWYTDGSSLLQEGQRKAGAAVTTETEVIWAKALPAGTSAQRAELIALTQALKMAEGKK |
| | LNVYTDSRYAFATAHIHGEIYRRRGWLTSEGKEIKNKDEILALLKALFLPKRLSIIHCPGHQKGHS |
| | AEARGNRMADQAARKAAITETPDTSTLLIENSSPSGGSKRTADGSEFEPKKKRKV |
| -N-TERMINAL | MKRTADGSEFESPKKKRKV |
| NLS | |
| (SEQ ID NO: | |
| 7547) | |
| CAS9(H840A) | DKKYSIGLDIGTNSVGWAVITDEYKVPSKKFKVLGNTDRHSIKKNLIGALLFDS |
| (MET MINUS) | GETAEATRLKRTARRRYTRRKNRICYLQEIFSNEMAKVDDSFFHRLEESFLVEE |
| (SEQ ID NO: | DKKHERHPIFGNIVDEVAYHEKYPTIYHLRKKLVDSTDKADLRLIYLALAHMI |
| 7562) | KFRGHFLIEGDLNPDNSDVDKLFIQLVQTYNQLFEENPINASGVDAKAILSARL |
| | SKSRRLENLIAQLPGEKKNGLFGNLIALSLGLTPNFKSNFDLAEDAKLQLSKDT |
| | YDDDLDNLLAQIGDQYADLFLAAKNLSDAILLSDILRVNTEITKAPLSASMIKR |
| | YDEHHQDLTLLKALVRQQLPEKYKEIFFDQSKNGYAGYIDGGASQEEFYKFIK |
| | PILEKMDGTEELLVKLNREDLLRKQRTFDNGSIPHQIHLGELHAILRRQEDFYP |
| | FLKDNREKIEKILTFRIPYYVGPLARGNSRFAWMTRKSEETITPWNFEEVVDKG |
| | ASAQSFIERMTNFDKNLPNEKVLPKHSLLYEYFTVYNELTKVKYVTEGMRKP |
| | AFLSGEQKKAIVDLLFKTNRKVTVKQLKEDYFKKIECFDSVEISGVEDRFNAS |
| | LGTYHDLLKIIKDKDFLDNEENEDILEDIVLTLTLFEDREMIEERLKTYAHLFDD |
| | KVMKQLKRRRYTGWGRLSRKLINGIRDKQSGKTILDFLKSDGFANRNFMQLI |
| | HDDSLTFKEDIQKAQVSGQGDSLHEHIANLAGSPAIKKGILQTVKVVDELVKV |
| | MGRHKPENIVIEMARENQTTQKGQKNSRERMKRIEEGIKELGSQILKEHPVEN |
| | TQLQNEKLYLYYLQNGRDMYVDQELDINRLSDYDVDAIVPQSFLKDDSIDNK |
| | VLTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLKS |
| | KLVSDFRKDFQFYKVREINNYHHAHDAYLNAVVGTALIKKYPKLESEFVYGD |
| | YKVYDVRKMIAKSEQEIGKATAKYFFYSNIMNFFKTEITLANGEIRKRPLIETN |
| | GETGEIVWDKGRDFATVRKVLSMPQVNIVKKTEVQTGGFSKESILPKRNSDKL |
| | IARKKDWDPKKYGGFDSPTVAYSVLVVAKVEKGKSKKLKSVKELLGITIMERS |
| | SFEKNPIDFLEAKGYKEVKKDLIIKLPKYSLFELENGRKRMLASAGELQKGNE |
| | LALPSKYVNFLYLASHYEKLKGSPEDNEQKQLFVEQHKHYLDEIIEQISEFSKR |
| | VILADANLDKVLSAYNKHRDKPIREQAENIIHLFTLTNLGAPAAFKYFDTTIDR |
| | KRYTSTKEVLDATLIHQSITGLYETRIDLSQLGGD |
| LINKER BETWEEN | SGGSSGGSSGSETPGTSESATPESSGGSSGGSS |
| CAS9 DOMAIN | |
| AND RT DOMAIN | |
| (33 AMINO ACIDS) | |
| (SEQ ID NO: | |
| 7563) | |
| MMLV_RT | TLNIEDEYRLHETSKEPDVSLGSTWLSDFPQAWAETGGMGLAVRQAPLIIPLK |
| D200N | ATSTPVSIKQYPMSQEARLGIKPHIQRLLDQGILVPCQSPWNTPLLPVKKPGTN |
| T330P | DYRPVQDLREVNKRVEDIHPTVPNPYNLLSGLPPSHQWYTVLDLKDAFFCLR |
| L603W | LHPTSQPLFAFEWRDPEMGISGQLTWTRLPQGFKNSPTLFNEALHRDLADFRI |
| T306K | QHPDLILLQYVDDLLLAATSELDCQQGTRALLQTLGNLGYRASAKKAQICQK |
| W313F | QVKYLGYLLKEGQRWLTEARKETVMGQPTPKTPRQLREFLGKAGFCRLFIPG |
| (SEQ ID NO: | FAEMAAPLYPLTKPGTLFNWGPDQQKAYQEIKQALLTAPALGLPDLTKPFELF |
| 7564) | VDEKQGYAKGVLTQKLGPWRRPVAYLSKKLDPVAAGWPPCLRMVAAIAVLT |
| | KDAGKLTMGQPLVILAPHAVEALVKQPPDRWLSNARMTHYQALLLDTDRVQ |
| | FGPVVALNPATLLPLPEEGLQHNCLDILAEAHGTRPDLTDQPLPDADHTWYTD |
| | GSSLLQEGQRKAGAAVTTETEVIWAKALPAGTSAQRAELIALTQALKMAEGK |
| | KLNVYTDSRYAFATAHIHGEIYRRRGWLTSEGKEIKNKDEILALLKALFLPKRL |
| | SIIHCPGHQKGHSAEARGNRMADQAARKAAITETPDTSTLLIENSSP |
| -C-TERMINAL | SGGSKRTADGSEFEPKKKRKV |
| Linker and NLS | |
| (SEQ ID NO: | |
| 7554) | |
| C Terminal NLS | KRTADGSEFEPKKKRKV |
| (SEQ ID NO: 7555 | |

TABLE 53
Amino acid sequences of an exemplary PE fusion protein and its individual components

| SEQ ID | | |
|---|---|---|
| NO. | DESCRIPTION | SEQUENCE |
| 7565 | Exemplary prime editor | MKRTADGSEFESPKKKRKVDKKYSIGLDIGTNSVGWAVITDEYKV |
| | [NLS]-[Cas9((R220K) | PSKKFKVLGNTDRHSIKKNLIGALLFDSGETAEATRLKRTARRR |
| | (R393K)(H839A)]- | YTRRKNRICYLQEIFSNEMAKVDDSFFHRLEESFLVEEDKKHER |
| | [linker]- | HPIFGNIVDEVAYHEKYPTIYHLRKKLVDSTDKADLRLIYLALAH |
| | [MMLV_RT(D200N)(T330P) | MIKFRGHFLIEGDLNPDNSDVDKLFIQLVQTYNQLFEENPINASG |
| | (L603W)(T306K)(W313F)]- | VDAKAILSARLSKSRKLENLIAQLPGEKKNGLFGNLIALSLGLTP |
| | [NLS] | NFKSNFDLAEDAKLQLSKDTYDDDLDNLLAQIGDQYADLFLAAK |
| | | NLSDAILLSDILRVNTEITKAPLSASMIKRYDEHHQDLTLLKALV |
| | | RQQLPEKYKEIFFDQSKNGYAGYIDGGASQEEFYKFIKPILEKMD |
| | | GTEELLVKLKREDLLRKQRTFDNGSIPHQIHLGELHAILRRQED |
| | | FYPFLKDNREKIEKILTFRIPYYVGPLARGNSRFAWMTRKSEETI |
| | | TPWNFEEVVDKGASAQSFIERMTNFDKNLPNEKVLPKHSLLYEY |
| | | FTVYNELTKVKYVTEGMRKPAFLSGEQKKAIVDLLFKTNRKVT |
| | | VKQLKEDYFKKIECFDSVEISGVEDRFNASLGTYHDLLKIIKDKD |
| | | FLDNEENEDILEDIVLTLTLFEDREMIEERLKTYAHLFDDKVMK |
| | | QLKRRRYTGWGRLSRKLINGIRDKQSGKTILDFLKSDGFANRNF |
| | | MQLIHDDSLTFKEDIQKAQVSGQGDSLHEHIANLAGSPAIKKGIL |
| | | QTVKVVDELVKVMGRHKPENIVIEMARENQTTQKGQKNSRER |
| | | MKRIEEGIKELGSQILKEHPVENTQLQNEKLYLYYLQNGRDMY |
| | | VDQELDINRLSDYDVDAIVPQSFLKDDSIDNKVLTRSDKNRGKSD |
| | | NVPSEEVVKKMKNYWRQLLNAKLITQRKFDNLTKAERGGLSEL |
| | | DKAGFIKRQLVETRQITKHVAQILDSRMNTKYDENDKLIREVKV |
| | | ITLKSKLVSDFRKDFQFYKVREINNYHHAHDAYLNAVVGTALIK |
| | | KYPKLESEFVYGDYKVYDVRKMIAKSEQEIGKATAKYFFYSNIM |
| | | NFFKTEITLANGEIRKRPLIETNGETGEIVWDKGRDFATVRKVLS |
| | | MPQVNIVKKTEVQTGGFSKESILPKRNSDKLIARKKDWDPKKYG |
| | | GFDSPTVAYSVLVVAKVEKGKSKKLKSVKELLGITIMERSSFEKN |
| | | PIDFLEAKGYKEVKKDLIIKLPKYSLFELENGRKRMLASAGELQ |
| | | KGNELALPSKYVNFLYLASHYEKLKGSPEDNEQKQLFVEQHKH |
| | | YLDEIIEQISEFSKRVILADANLDKVLSAYNKHRDKPIREQAENII |
| | | HLFTLTNLGAPAAFKYFDTTIDRKRYTSTKEVLDATLIHQSITGL |
| | | YETRIDLSQLGGDSGGSSGGSKRTADGSEFESPKKKRKVSGGSSG |
| | | GSTLNIEDEYRLHETSKEPDVSLGSTWLSDFPQAWAETGGMGLAVRQAP |
| | | LIIPLKATSTPVSIKQYPMSQEARLGIKPHIQRLLDQGILVPCQSPWNTPLL |
| | | PVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPT |
| | | LFNEALHRDLADFRIQHPDLILLQYVDDLLLAATSELDCQQGTRALLQTL |
| | | GNLGYRASAKKAQICQKQVKYLGYLLKEGQRWLTEARKETVMGQPTPK |
| | | TPRQLREFLGKAGFCRLFIPGFAEMAAPLYPLTKPGTLFNWGPDQQKAY |
| | | QEIKQALLTAPALGLPDLTKPFELFVDEKQGYAKGVLTQKLGPWRRPVA |
| | | YLSKKLDPVAAGWPPCLRMVAAIAVLTKDAGKLTMGQPLVILAPHAVEA |
| | | LVKQPPDRWLSNARMTHYQALLLDTDRVQFGPVVALNPATLLPLPEEGL |
| | | QHNCLDILAEAHGTRPDLTDQPLPDADHTWYTDGSSLLQEGQRKAGAA |
| | | VTTETEVIWAKALPAGTSAQRAELIALTQALKMAEGKKLNVYTDSRYAFA |
| | | TAHIHGEIYRRRGWLTSEGKEIKNKDEILALLKALFLPKRLSIIHCPGHQK |
| | | GHSAEARGNRMADQAARKAAITETPDTSTLLIENSSPSGGSKRTADGSEF |
| | | ESPKKKRKVGSGPAAKRVKLD |
| | | KEY: |
| | | N-terminal bipartiteSV40NLS |
| | | CAS9(R221K N394K H840A) |
| | | SGGSx2-met-bpSV40NLS-SGGSx2 LINKER |
| | | M-MLV D200N T306K W313F T330P L603W REVERSE TRANSCRIPTASE |
| | | C-terminal linker- NLS1 |
| | | C-terminal linker-NLS2 |
| 7566 | Exemplary prime editor | KRTADGSEFESPKKKRKVDKKYSIGLDIGTNSVGWAVITDEYKVPSK |
| | [NLS]-[Cas9((R220K) | KFKVLGNTDRHSIKKNLIGALLFDSGETAEATRLKRTARRRYTRRKN |
| | (R393K)(H839A)]- | RICYLQEIFSNEMAKVDDSFFHRLEESFLVEEDKKHERHPIFGNIVDE |
| | [linker]- | VAYHEKYPTIYHLRKKLVDSTDKADLRLIYLALAHMIKFRGHFLIEG |
| | [MMLV_RT(D200N)(T330P) | DLNPDNSDVDKLFIQLVQTYNQLFEENPINASGVDAKAILSARLSKS |
| | (L603W)(T306K)(W313F)]- | RKLENLIAQLPGEKKNGLFGNLIALSLGLTPNFKSNFDLAEDAKLQL |
| | [NLS] without N | SKDTYDDDLDNLLAQIGDQYADLFLAAKNLSDAILLSDILRVNTEIT |
| | terminal methionine | KAPLSASMIKRYDEHHQDLTLLKALVRQQLPEKYKEIFFDQSKNGY |
| | | AGYIDGGASQEEFYKFIKPILEKMDGTEELLVKLKREDLLRKQRTFD |
| | | NGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKA |
| | | IVDLLFKTNRKVTVKQLKEDYFKKIECFDSVEISGVEDRFNASLGTY |
| | | HDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFA |
| | | NRNFMQLIHDDSLTFKEDIQKAQVSGQGDSLHEHIANLAGSPAIKKG |
| | | ILQTVKVVDELVKVMGRHKPENIVIEMARENQTTQKGQKNSRERM |
| | | KRIEEGIKELGSQILKEHPVENTQLQNEKLYLYYLQNGRDMYVDQE |
| | | LDINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPK |
| | | YSLFELENGRKRMLASAGELQKGNELALPSKYVNFLYLASHYEKLK |
| | | GSPEDNEQKQLFVEQHKHYLDEIIEQISEFSKRVILADANLDKVLSAY |
| | | NKHRDKPIREQAENIIHLFTLTNLGAPAAFKYFDTTIDRKRYTSTKEV |
| | | LDATLIHQSITGLYETRIDLSQLGGDSGGSSGGSKRTADGSEFESPKK |
| | | KRKVSGGSSGGSTLNIEDEYRLHETSKEPDVSLGSTWLSDFPQAWAE |
| | | TGGMGLAVRQAPLIIPLKATSTPVSIKQYPMSQEARLGIKPHIQRLLD |
| | | QGILVPCQSPWNTPLLPVKKPGTNDYRPVQDLREVNKRVEDIHPTVP |
| | | NPYNLLSGLPPSHQWYTVLDLKDAFFCLRLHPTSQPLFAFEWRDPE |
| | | MGISGQLTWTRLPQGFKNSPTLFNEALHRDLADFRIQHPDLILLQYV |
| | | DDLLLAATSELDCQQGTRALLQTLGNLGYRASAKKAQICQKQVKY |
| | | LGYLLKEGQRWLTEARKETVMGQPTPKTPRQLREFLGKAGFCRLFIP |
| | | GFAEMAAPLYPLTKPGTLFNWGPDQQKAYQEIKQALLTAPALGLPD |
| | | LTKPFELFVDEKQGYAKGVLTQKLGPWRRPVAYLSKKLDPVAAGW |
| | | PPCLRMVAAIAVLTKDAGKLTMGQPLVILAPHAVEALVKQPPDRWL |
| | | SNARMTHYQALLLDTDRVQFGPVVALNPATLLPLPEEGLQHNCLDI |
| | | LAEAHGTRPDLTDQPLPDADHTWYTDGSSLLQEGQRKAGAAVTTE |
| | | TEVIWAKALPAGTSAQRAELIALTQALKMAEGKKLNVYTDSRYAFA |
| | | TAHIHGEIYRRRGWLTSEGKEIKNKDEILALLKALFLPKRLSIIHCPGH |
| | | QKGHSAEARGNRMADQAARKAAITETPDTSTLLIENSSPSGGSKRTA |
| | | DGSEFESPKKKRKVGSGPAAKRVKLD |
| 7547 | -N-terminal bpSV40NLS | MKRTADGSEFESPKKKRKV |
| 7593 | -CAS9 (R221K N394K | DKKYSIGLDIGTNSVGWAVITDEYKVPSKKFKVLGNTDRHSIKKNLI |
| | H840A) | GALLFDSGETAEATRLKRTARRRYTRRKNRICYLQEIFSNEMAKVD |
| | | DSFFHRLEESFLVEEDKKHERHPIFGNIVDEVAYHEKYPTIYHLRKKL |
| | | VDSTDKADLRLIYLALAHMIKFRGHFLIEGDLNPDNSDVDKLFIQLV |
| | | QTYNQLFEENPINASGVDAKAILSARLSKSRKLENLIAQLPGEKKNG |
| | | LFGNLIALSLGLTPNFKSNFDLAEDAKLQLSKDTYDDDLDNLLAQIG |
| | | DQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIK |
| | | PILEKMDGTEELLVKLKREDLLRKQRTFDNGSIPHQIHLGELHAILRR |
| | | QEDFYPFLKDNREKIEKILTFRIPYYVGPLARGNSRFAWMTRKSEETI |
| | | TPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFK |
| | | EDIQKAQVSGQGDSLHEHIANLAGSPAIKKGILQTVKVVDELVKVM |
| | | GRHKPENIVIEMARENQTTQKGQKNSRERMKRIEEGIKELGSQILKE |
| | | HPVENTQLQNEKLYLYYLQNGRDMYVDQELDINRLSDYDVDAIVP |
| | | QSFLKDDSIDNKVLTRSDKNRGKSDNVPSEEVVKKMKNYWRQLLN |
| | | AKLITQRKFDNLTKAERGGLSELDKAGFIKRQLVETRQITKHVAQIL |
| | | DSRMNTKYDENDKLIREVKVITLKSKLVSDFRKDFQFYKVREINNY |
| | | HHAHDAYLNAVVGTALIKKYPKLESEFVYGDYKVYDVRKMIAKSE |
| | | QEIGKATAKYFFYSNIMNFFKTEITLANGEIRKRPLIETNGETGEIVW |
| | | DKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| 7594 | -SGGSx2-bpSV40NLS- | SGGSSGGSKRTADGSEFESPKKKRKVSGGSSGGS |
| | SGGSx2 linker | |
| 7564 | -MMLV_RT D200N | TLNIEDEYRLHETSKEPDVSLGSTWLSDFPQAWAETGGMGLAVRQA |
| | T330P L603W T306K | PLIIPLKATSTPVSIKQYPMSQEARLGIKPHIQRLLDQGILVPCQSPWN |
| | W313F | TPLLPVKKPGTNDYRPVQDLREVNKRVEDIHPTVPNPYNLLSGLPPS |
| | | HQWYTVLDLKDAFFCLRLHPTSQPLFAFEWRDPEMGISGQLTWTRL |
| | | PQGFKNSPTLFNEALHRDLADFRIQHPDLILLQYVDDLLLAATSELD |
| | | CQQGTRALLQTLGNLGYRASAKKAQICQKQVKYLGYLLKEGQRWL |
| | | TEARKETVMGQPTPKTPRQLREFLGKAGFCRLFIPGFAEMAAPLYPL |
| | | TKPGTLFNWGPDQQKAYQEIKQALLTAPALGLPDLTKPFELFVDEK |
| | | QGYAKGVLTQKLGPWRRPVAYLSKKLDPVAAGWPPCLRMVAAIA |
| | | VLTKDAGKLTMGQPLVILAPHAVEALVKQPPDRWLSNARMTHYQA |
| | | LLLDTDRVQFGPVVALNPATLLPLPEEGLQHNCLDILAEAHGTRPDL |
| | | TDQPLPDADHTWYTDGSSLLQEGQRKAGAAVTTETEVIWAKALPA |
| | | GTSAQRAELIALTQALKMAEGKKLNVYTDSRYAFATAHIHGEIYRR |
| | | RGWLTSEGKEIKNKDEILALLKALFLPKRLSIIHCPGHQKGHSAEAR |
| | | GNRMADQAARKAAITETPDTSTLLIENSSP |
| 7595 | C-terminal linker-NLS | SGGSKRTADGSEFESPKKKRKV |
| 7596 | C-terminal linker-NLS2 | GSGPAAKRVKLD |

### PEgRNA for Editing of USH2A Gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

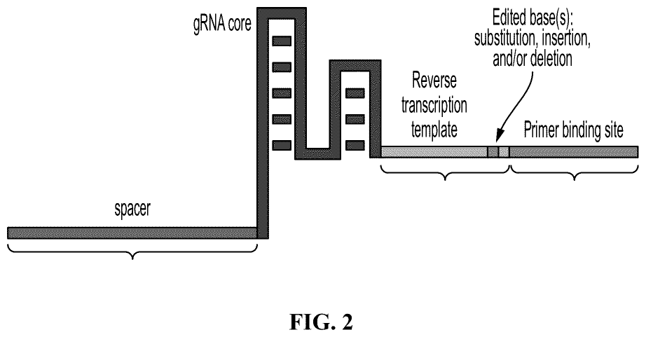

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene, e.g., an USH2A gene. In some embodiments, the editing template comprises substantial or partial complementarity to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template. and a PBS. In some embodiments, the PEgRNA comprises, from 5′ to 3′: an extension arm, a spacer, and a gRNA core. In some embodiments, the PEgRNA comprises, from 5′ to 3′: an editing template, a PBS, a spacer, and a gRNA core.

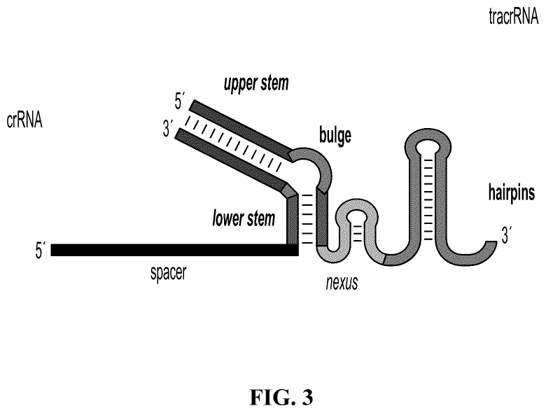

In some embodiments, a PEgRNA comprises a single polynucleotide molecule that comprises the spacer, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a spacer sequence comprises a region that has substantial complementarity to a search target sequence on the target strand of a double stranded target DNA, e.g., an USH2A gene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from about least 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 17 to 22 nucleotides in length, e.g., about 17, 18, 19, 20, 21, or 22 nucleotides in length. In some embodiments, the spacer is 20 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that comprises complementarity to, and can hybridize with a free 3′ end of a single stranded DNA in the target gene (e.g., the USH2A gene) generated by nicking with a prime editor at the nick site on the PAM strand. The length of the PBS sequence may vary depending on, e.g., the prime editor components, the search target sequence and other components of the PEgRNA. In some embodiments, the length of the primer binding site (PBS) is about 3 to 19 nucleotides in length. In some embodiments, the PBS is about 3 to 17 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is 8 to 17 nucleotides in length. In some embodiments, the PBS is 8 to 16 nucleotides in length. In some embodiments, the PBS is 8 to 15 nucleotides in length. In some embodiments, the PBS is 8 to 14 nucleotides in length. In some embodiments, the PBS is 8 to 13 nucleotides in length. In some embodiments, the PBS is 8 to 12 nucleotides in length. In some embodiments, the PBS is 8 to 11 nucleotides in length. In some embodiments, the PBS is 8 to 10 nucleotides in length. In some embodiments, the PBS is 8 or 9 nucleotides in length. In some embodiments, the PBS is 16 or 17 nucleotides in length. In some embodiments, the PBS is 15 to 17 nucleotides in length. In some embodiments, the PBS is 14 to 17 nucleotides in length. In some embodiments, the PBS is 13 to 17 nucleotides in length. In some embodiments, the PBS is 12 to 17 nucleotides in length. In some embodiments, the PBS is 11 to 17 nucleotides in length. In some embodiments, the PBS is 10 to 17 nucleotides in length. In some embodiments, the PBS is 9 to 17 nucleotides in length. In some embodiments, the PBS is about 7 to 15 nucleotides in length. In some embodiments, the PBS is 6, 7, 8, 9, 10, 11, 12, 13, 14, 15, 16, 17, 18, or 19 nucleotides in length. In some embodiments, the PBS is 8 to 14 nucleotides in length. For example, the PBS can be 8, 9, 10, 11, 12, 13, or 14 nucleotides in length. In some embodiments, the PBS is 11 or 12 nucleotides in length. In some embodiments, the PBS is 11 to 13 nucleotides in length. In some embodiments, the PBS is 11 to 14 nucleotides in length. In some cases, a PBS length of no more than 3 nucleotides less than the PEgRNA spacer is chosen. For example, for PEgRNA spacers that are 16 to 22 nucleotides in length, a PBS length of up to 19 nucleotides, e.g., 3 to 19, 5 to 19, or 7 to 19 nucleotides, may be chosen. In some embodiments, the PBS is 5 to 19 nucleotides in length.

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., the USH2A gene). In some embodiments, the PBS is perfectly complementary, or 100% complementary, to a region of the edit strand of the target gene (e.g., the USH2A gene).

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length.

In some embodiments, the RTT is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In some embodiments, the RTT is 10 to 110 nucleotides in length. In some embodiments, the RTT is 10 to 109, 10 to 108, 10 to 107, 10 to 106, 10 to 105, 10 to 104, 10 to 103, 10 to 102, or 10 to 101 nucleotides in length. In some embodiments, the RTT is from 14 to 34 nucleotides in length. In some embodiments, the RTT is from 18 to 22 nucleotides in length. In some embodiments, the RTT is at least 8 and no more than 50 nucleotides in length. In some embodiments, the RTT is at least 8 and no more than 25 nucleotides in length. In some embodiments, the RTT is about 10 to about 20 nucleotides in length. In some embodiments, the RTT is about 11, 12, 13, 14, 15, 16, 17, 18, or 19 nucleotides in length. In some embodiments, the RTT is 11 to 17 nucleotides in length. In some embodiments, the RTT is 12 to 17 nucleotides in length. In some embodiments, the RTT is 12 to 16 nucleotides in length. In some embodiments, the RTT is 13 to 17 nucleotides in length. In some embodiments, the RTT is 11, 12, 13, 14, 15, 16, or 17 nucleotides in length. In some embodiments the RTT is 12 nucleotides in length. In some embodiments the RTT is 16 nucleotides in length. In some embodiments the RTT is 17 nucleotides in length.

In some embodiments, the RTT has a length of 44 nucleotides or less. In some embodiments, the RTT has a length of 34 nucleotides or less. In some embodiments, the RTT has a length of 22 nucleotides or less.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated int the target gene. In some embodiments, the editing template comprises a nucleotide sequence comprising about 85% to about 95% complementarity to an editing target sequence in the edit strand in the target gene (e.g., the USH2A gene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the USH2A gene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 1, at least 2, at least 3, at least 4, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the USH2A gene to be edited. Position of the intended nucleotide edit(s) relevant to other components of the PEgRNA, or to particular nucleotides (e.g., mutations) in the USH2A target gene may vary. In some embodiments, the nucleotide edit is in a region of the PEgRNA corresponding to or homologous to the protospacer sequence. In some embodiments, the nucleotide edit is in a region of the PEgRNA corresponding to a region of the USH2A gene outside of the protospacer sequence.

By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene may be referred to based on the position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template may be referred to by aligning the editing template with the partially complementary editing target sequence on the edit strand and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand may be referred to as at positions +1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity may also be referred to as position 0 in the editing template, the nucleotides in the editing template corresponding to the nucleotides at positions +1, +2, +3, +4, . . . , +n on the PAM strand of the double stranded target DNA may also be referred to as at positions +1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions +1, +2, +3, +4, . . . , +n are 5′ of position 0 and positions −1, −2, −3, −4, . . . , −n are 3′ of position 0 in the editing template. In some embodiments, an intended nucleotide edit is at position +n of the editing template relative to position 0. Accordingly, the intended nucleotide edit may be incorporated at position +n of the PAM strand of the double stranded target DNA (and subsequently, the target strand of the double stranded target DNA) by prime editing wherein n is an integer no less than 0. The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the USH2A and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand). As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). In some embodiments, the nick-to-edit distance is 2 to 106 nucleotides. In some embodiments, the nick-to-edit distance is 2 to 105, 2 to 104, 2 to 103, 2 to 102, 2 to 101, 2 to 100, 2 to 99, 2 to 98, or 2 to 97 nucleotides. In some embodiments, the nick-to-edit distance is 2 to 90, 2 to 80, 2 to 70, 2 to 60, 2 to 50, 2 to 40, or 2 to 30 nucleotides. In some embodiments, the nick-to-edit distance is 2 to 25, 2 to 20, 2 to 15, or 2 to 10 nucleotides. In some embodiments, the nick-to-edit distance is 2, 3, 4, 5, 6, or 7 nucleotides in length.

The RTT length and the nick-to-edit distance relate to the length of the portion of the RTT that is upstream of (i.e., 5′ to) the 5′-most edit in the RTT and is complementary to the edit strand. In some embodiments, the editing template comprises at least 4 contiguous nucleotides of complementarity with the edit strand wherein the at least 4 nucleotides contiguous are located upstream of the 5′ most edit in the editing template. In some embodiments, the editing template comprises at least 5, 6, 7, 8, 9, 10, 11, 12, 13, 14, 15, 16, 17, 18, 19, or more contiguous nucleotides of complementarity with the edit strand wherein the at least 5, 6, 7, 8, 9, 10, 11, 12, 13, 14, 15, 16, 17, 18, 19, or more contiguous nucleotides are located upstream of the 5′ most edit in the editing template. In some embodiments, the editing template comprises 20-25, 25-30, 30-35, 35-40, 45-45, or 45-50 contiguous nucleotides of complementarity with the edit strand wherein the 20-25, 25-30, 30-35, 35-40, 45-45, or 45-50 or more contiguous nucleotides are located upstream of the 5′ most edit in the editing template. In some embodiments, the editing template comprises 9-14 contiguous nucleotides of complementarity with the edit strand wherein the 9-14 contiguous nucleotides are located upstream of the 5′ most edit in the editing template. In some embodiments, the editing template comprises 6-10 contiguous nucleotides of complementarity with the edit strand wherein the 6-10 contiguous nucleotides are located upstream of the 5′ most edit in the editing template. In some embodiments, the editing template comprises 10 contiguous nucleotides of complementarity with the edit strand wherein the 10 contiguous nucleotides are located upstream of the 5′ most edit in the editing template. In some embodiments, the editing template comprises 9 contiguous nucleotides of complementarity with the edit strand wherein the 9 contiguous nucleotides are located upstream of the 5′ most edit in the editing template.

When referred to within the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the PBS. In some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the PBS.

The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target USH2A gene and the and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0 base pair from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template can comprise a second edit relative to a target sequence. The second edit can be designed to mutate or otherwise silence a PAM sequence such that a corresponding nucleic acid guided nuclease or CRISPR nuclease is no longer able to cleave the target sequence (such edits referred to as “PAM silencing edits).

Without wishing to be bound by any particular theory. PAM silencing edits may prevent the Cas, e.g., Cas9, nickase, from re-nicking the edit strand before the edit is incorporated in the target strand, therefore improving prime editing efficiency. In some embodiments, a PAM silencing edit is a synonymous edit that does not alter the amino acid sequence encoded by the target gene after incorporation of the edit.

In some embodiments, the length of the editing template is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides longer than the nick to edit distance. In some embodiments, for example, the nick to edit distance is 8 nucleotides, and the editing template is 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, or 10 to 80 nucleotides in length. In some embodiments, the nick to edit distance is 22 nucleotides, and the editing template is 24 to 28, 24 to 30, 24 to 32, 24 to 34, 24 to 36, 24 to 37, 24 to 38, 24 to 40, 24 to 45, 24 to 50, 24 to 55, 24 to 60, 24 to 65, 24 to 70, 24 to 75, 24 to 80, 24 to 85, 24 to 90, 24 to 95, 24 to 100, 24 to 105, 24 to 100, 24 to 105, or 24 to 110 nucleotides in length.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In sonic embodiments, the editing template comprises an uracil at the first nucleobase position (e.g., for a PEgR_NA follow ing 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “-first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g., by reverse transcription) to replace an editing target sequence in the target gene. In some embodiments, the editing target sequence in the edit strand of the target gene is replaced by the newly synthesized strand, and the nucleotide edit(s) are incorporated in the region of the target gene. In some embodiments, the target gene is an USH2A gene. In some embodiments, the editing template of the PEgRNA encodes a newly synthesized single stranded DNA that comprises a wild type USH2A gene sequence. In some embodiments, the newly synthesized DNA strand replaces the editing target sequence in the target USH2A gene, wherein the editing target sequence (or the endogenous sequence complementary to the editing target sequence on the target strand of the USH2A gene) comprises a mutation or a nucleotide alteration compared to a reference gene, e.g., a wild type USH2A gene. In some embodiments, the mutation is associated with nsRP. In some embodiments, the mutation is associated with Usher syndrome type 2. In some embodiments, the newly synthesized DNA strand replaces the editing target sequence in a target USH2A gene.

In some embodiments, the editing target sequence comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28. exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57. exon 58. exon 59, exon 60, exon 61, exon 62, exon 63, exon 64, exon 65, exon 66, exon 67, exon 68, exon 69, exon 70, exon 71, or exon 72 of the USH2A gene as compared to a wild type USH2A gene. In some embodiments, the editing target sequence comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 of the USH2A gene as compared to a wild type USH2A gene. In some embodiments, the editing target sequence comprises a mutation at a splice site (i.e., an intron/exon junction) of the USH2A gene as compared to a wild type USH2A gene. In some embodiments, the editing target sequence comprises a mutation in an intron of the USH2A gene as compared to a wild type USH2A gene.

In some embodiments, the editing template comprises one or more intended nucleotide edits compared to the sequence on the target strand of the USH2A gene that is complementary to the editing target sequence. In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits. In some embodiments, incorporation of the one or more intended nucleotide edits corrects the mutation in the editing target sequence to wild type nucleotides at corresponding positions in the USH2A gene. As used herein, “correcting” a mutation means restoring a wild type sequence at the place of the mutation in the double stranded target DNA, e.g. target gene, by prime editing. In some embodiments, the editing template comprises a wild type USH2A gene sequence.

In some embodiments, incorporation of the one or more intended nucleotide edits does not correct the mutation in the editing target sequence to wild type sequence, but allows for expression of a functional usherin protein encoded by the USH2A gene. For example, in some embodiments, incorporation of the one or more intended nucleotide edits results in one or more codons that are different from a wild type codon but encode one or more amino acids same as the wild type usherin protein. In some embodiments, incorporation of the one or more intended nucleotide edits results in one or more codons that encode one or more amino acids different from the wild type usherin protein but allows for expression of a functional usherin protein.

In some embodiments, the editing template comprises one or more intended nucleotide edits compared to the sequence on the target strand of the target USH2A gene that is complementary to the editing target sequence, wherein the one or more intended nucleotide edits is a single nucleotide substitution, polynucleotide substitution, nucleotide insertion, or nucleotide deletion. In some embodiments, the intended nucleotide edit in the editing template comprises a single nucleotide substitution, polynucleotide substitution, nucleotide insertion, or nucleotide deletion compared to the sequence on the target strand of the target USH2A gene that is complementary to the editing target at a position corresponding to a mutation in target gene, wherein the editing target sequence is on the sense strand of the target gene. In some embodiments, the intended nucleotide edit in the editing template comprises a single nucleotide substitution, polynucleotide substitution, nucleotide insertion, or nucleotide deletion compared to the sequence on the target strand of the target gene that is complementary to the editing target at a position corresponding to a mutation in target gene, wherein the editing target sequence is on the antisense strand of the target USH2A gene.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a CpfI-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “tipper stem” following the lower stem, where the lower stem and upper stem may be connected by a “bulge” comprising unpaired RNAs. The gRNA core may further comprise a “nexus” distal from the spacer sequence, followed by a hairpin structure e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, a prime editing system comprises a prime editor and a PEgRNA, wherein the prime editor comprises a SpCas9 nickase variant thereof, and the gRNA core of the PEgRNA comprises the sequence:
GUUUUAGAGCUAGAAAUAGCAAGUIJAAAAUAAOOCLUAUCCGIAJCAACUUGAAAAAG UGGCACCGAOUCGGUGC (SEQ ID NO: 7544);
GUUUGAGCACUAGAAAUAGCAAGUUUAAAUAAGGCUAGUCCGUUAUCAACUUGA AAAAGUGGGACCCGAGUCCGGUCC (SEQ ID NO: 7545), or
GUUUAAGAGCUAUGCUGGAACACCAUAGCAAGUUUAAAUAAGGCUAGUCCGUUAUCAA CUUGAAAAACGUGGCACCGACGUCGGUCGC (SEQ ID NO: 7546). In some embodiments, the gRNA core comprises the sequence GUUUUAGAGCUAGAAAUACCAAGUUAAAAUAAGGCUAGUCCGUUAUCAACUUGAAAAAG UGGICACCGAGUCGGUGC (SEQ ID NO: 7544). Any gRNA core sequences known in the art are also contemplated in the prime editing; compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). In some embodiments, a ngRNA comprises a spacer (referred to as a ngRNA spacer or ng spacer) and a gRNA core, wherein the spacer of the ngRNA comprises a region of complementarity to the edit strand, and wherein the gRNA core can interact with a Cas, e.g., Cas9, of a prime editor. Without wishing to be bound by any particular theory, an ngRNA may bind to the edit strand and direct the Cas nickase to generate a nick on the non-edit strand (or target strand). In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

A prime editing system comprising a PEgRNA (or one or more polynucleotide encoding the PEgRNA) and a prime editor protein (or one or more polynucleotides encoding the prime editor), may be referred to as a PE2 prime editing system and the corresponding editing approach referred to as PE2 approach or PE2 strategy. A PE2 system does not contain a ngRNA. A prime editing system comprising a PEgRNA (or one or more polynucleotide encoding the PEgRNA), a prime editor protein (or one or more polynucleotides encoding the prime editor), and a ngRNA (or one or more polynucleotides encoding the ngRNA) may be referred to as a “PE3” prime editing system. In some embodiments, an ngRNA spacer sequence is complementary to a portion of the edit strand that includes the intended nucleotide edit, and may hybridize with the edit strand only after the edit has been incorporated on the edit strand. Such ngRNA may be referred to a “PE3b” ngRNA, and the prime editing system a PE3b prime editing system.

In some embodiments, a PEgRNA or a nick guide RNA (ngRNA) can be chemically synthesized, or can be assembled or cloned and transcribed from a DNA sequence, e.g., a plasmid DNA sequence, or by any RNA oligonucleotide synthesis method known in the art. In some embodiments, a DNA sequence that encodes a PEgRNA (or ngRNA) can be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) can be designed to append a nucleotide G at the 5′ end. Accordingly, in some embodiments, the PEgRNA (or nick guide RNA) can comprise an appended nucleotide G at the 5′ end. In some embodiments, a DNA sequence that encodes a PEgRNA (or nick guide RNA) can be designed to append a sequence that enhances transcription, e.g., a Kozak sequence, at the 5′ end. In some embodiments, a DNA sequence that encodes a PEgRNA (or nick guide RNA) can be designed to append the sequence CACC or CCACC at the 5′ end. Accordingly, in some embodiments, the PEgRNA (or nick guide RNA) can comprise an appended sequence CACC or CCACC at the 5′ end. In some embodiments, a DNA sequence that encodes a PEgRNA (or nick guide RNA) can be designed to append the sequence TTT, TTTT, TTTTT, TTTTTT, TTTTTTT at the 3′ end. Accordingly, in some embodiments, the PEgRNA (or nick guide RNA) can comprise an appended sequence UUU, UUUU, UUUUU, UUUUUU, or UUUUUUU at the 3′ end.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. In some embodiments, such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence.

A ngRNA protospacer may be in close proximity to the PEgRNA spacer or may be upstream or downstream of the PEgRNA spacer. In some embodiments, the distance generated by the PEgRNA nick site and the ngRNA nick site (referred to as the nick-to-nick distance) is about 3 to about 100 nucleotides. In some embodiments, the distance generated by the PEgRNA nick site and the ngRNA nick site (referred to as the nick-to-nick distance) is about 4-90, 4-80, 4-70, 4-60, 4-50, 4-40, 4-30, 4-20, or 4-10 nucleotides. In some embodiments, the distance generated by the PEgRNA nick site and the ngRNA nick site (referred to as the nick-to-nick distance) is about 10-20, 20-30, 30-40, 40-50, 50-60, 60-70, 70-80,80-90, or 90-100 nucleotides. In some embodiments, the nick-to-nick distance is about 4-88 nucleotides. In some embodiments, the nick-to-nick distance is about 4-72 nucleotides. In some embodiments, the nick-to-nick distance is about 4-61 nucleotides. In some embodiments, the nick-to-nick distance is about 61-72 nucleotides. In some embodiments, the nick-to-nick distance is about 61-88 nucleotides. In some embodiments, the nick-to-nick distance is about 72-88 nucleotides. In some embodiments, the nick-to-nick distance is about 4-7 nucleotides. In some embodiments, the nick-to-nick distance is 4, 5, 6. or 7 nucleotides. In some embodiments, the nick-to-nick distance is about 41-96 nucleotides. In some embodiments, the nick-to-nick distance is about 41-82 nucleotides. In some embodiments, the nick-to-nick distance is about 41-44 nucleotides. In some embodiments, the nick-to-nick distance is about 44-82 nucleotides. In some embodiments, the nick-to-nick distance is about 44-96 nucleotides. In some embodiments, the nick-to-nick distance is about 82-96 nucleotides. In some embodiments, the nick-to-nick distance is 41, 44, 82, or 96 nucleotides. In some embodiments, the intended nucleotide edit is incorporated within about 1-10 nucleotides of the position corresponding to the PAM of the ng search target sequence.

The gRNA core of a PEgRNA or ngRNA can be any gRNA scaffold sequence that is capable of interacting with a Cas protein that recognizes the corresponding PAM of the PEgRNA or ngRNA. In some embodiments, gRNA core of a PEgRNA or a ngRNA comprises a sequence selected from SEQ ID Nos: 7544-7545.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in the disclosure may further comprise nucleotides added to the 5′ of the PEgRNAs. In some embodiments, the PEgRNA further comprises 1, 2, or 3 additional nucleotides added to the 5′ end. The additional nucleotides can be guanine, cytosine, adenine, or uracil. In some embodiments, the additional nucleotide at the 5′ end of the PEgRNA is a guanine or cytosine. In some embodiments, the additional nucleotides can be chemically or biologically modified.

In some embodiments, the PEgRNAs provided in the disclosure may further comprise nucleotides to the 3′ of the PEgRNAs. In some embodiments, the PEgRNA further comprises 1, 2, or 3 additional nucleotides to the 3′ end. The additional nucleotides can be guanine, cytosine, adenine, or uracil. In some embodiments, the additional nucleotides at the 3′ end of the PEgRNA is a polynucleotide comprising at least 1 uracil. In some embodiments, the additional nucleotides can be chemically or biologically modified.

In some embodiments, a PEgRNA or ngRNA is produced by transcription from a template nucleotide, for example, a template plasmid. In some embodiments, a polynucleotide encoding the PEgRNA or ngRNA is appended with one or more additional nucleotides that improves PEgRNA or ngRNA function or expression, e.g., expression from a plasmid that encodes the PEgRNA or ngRNA. In some embodiments, a polynucleotide encoding a PEgRNA or ngRNA is appended with one or more additional nucleotides at the 5′ end or at the 3′ end. In some embodiments, the polynucleotide encoding the PEgRNA or ngRNA is appended with a guanine at the 5′ end, for example, if the first nucleotide at the 5′ end of the spacer is not a guanine. In some embodiments, a polynucleotide encoding the PEgRNA or ngRNA is appended with nucleotide sequence CACC at the 5′ end. In some embodiments, the polynucleotide encoding the PEgRNA or ngRNA is appended with additional nucleotide sequence TTTTTT, TTTTTTT, TTTTT, or TTTT at the 3′ end. In some embodiments, the PEgRNA or ngRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises an adenine at the 3′ end, for example, if the last nucleotide at the 3′ end of the PBS is a thymine. In some embodiments, the PEgRNA or ngRNA further comprises nucleotide sequence UUUUUUU, UUUUUU, UUUUU, or UUUU at the 3′ end.

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, the PEgRNA or ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 3, the gRNA core of a PEgRNA may comprise one or more regions of a base paired lower stem, a base paired upper stem. where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper sten, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a ′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition, complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA may be delivered simultaneously. For example, in some embodiments, a polynucleotide encoding the prime editor and a polynucleotide encoding a PEgRNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the AREs may comprise 50 to 150 nucleotides in length. In some embodiments, the AREs may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Unless otherwise indicated, references to nucleotide positions in human chromosomes are as set forth in human genome assembly consortium Human build 38 (GRCh38), GenBank accession GCF_000001405.38.

In some embodiments, the editing target sequence comprises a mutation that is in exon 13 of the USI-12A gene as compared to a wild type USH2A gene. In some embodiments, the editing target sequence comprises a mutation that is located between positions 216246995 and 216247195 of human chromosome 1. In some embodiments, the editing target sequence comprises a mutation that results in a E767 frameshift (E767fs) mutation in the usherin protein encoded by the USH2A gene as compared to a wild type usherin protein as set forth in SEQ ID NO:7598. In some embodiments, the editing target sequence comprises a deletion of nucleotide G at a position corresponding to position 216247095 of human chromosome 1 (c.2299de1G mutation) as compared to a wild type USH2A gene. In some embodiments, the editing target sequence comprises a deletion of nucleotide C at a position corresponding to position 216247095 of human chromosome 1 as compared to a wild type USH2A gene.

Exemplary combinations of PEgRNA components, e.g., spacer, PBS, and edit template/RTT, as well as combinations of each PEgRNA and corresponding ngRNA(s) are provided in Tables 1-44. Tables 1-44 each contain three columns. The left column is the sequence number. The middle column provides the sequence of the component, labeled with a SEQ ID NO where required by ST.26 standard. Although all the sequences provided in Tables 1-44 are RNA sequences, “T” is used instead of a “U” in the sequences for consistency with the ST.26 standard. The right column contains a description of the sequence. All of the PEgRNAs in Tables 1-19 are designed to correct a c.2299delG mutation in the USH2a gene. However, PEgRNA containing an RTT described with with a dual correction annotation in the right column are designed to also be capable of correcting a c.2276 G->T mutation in the USH2a gene. Similarly, all the PEgRNAs in Tables 20-44 are designed to correct a c.2276 G->T mutation in the USH2a gene, with PEgRNAs containing a dual correction annotated RTT designed to be capable of correcting a c.2276 G->T and/or a c2299delG mutation in the USH2a gene.

The PEgRNAs exemplified in Tables 1-44 comprise: (a) a spacer comprising at its 3′ end a sequence corresponding to a listed PEgRNA spacer sequence; (b) a gRNA core capable of complexing with a Cas9 protein, and (c) an extension arm comprising: (i) an editing template comprising at its 3′ end any RTT sequence from the same table as the PEgRNA spacer, and (ii) a prime binding site (PBS) comprising at its 5′ end any PBS sequence from the same table as the PEgRNA spacer. The PEgRNA spacer can be, for example, 17-22 nucleotides in length. The PEgRNA spacers in Tables 1-44 are annotated with their PAM sequence(s), enabling the selection of a prime editor comprising an appropriate Cas9 protein. The editing template can be referred to as a reverse transcription template (RTT). The editing template can encode wildtype USH2a gene sequence (annotated as simply RTT in Tables 1-44). Alternatively, the editing template can encode one or more synonymous mutations relative to the wildtype USH2a gene. The one or more synonymous mutations can be PAM silencing mutations. RTT encoding synonymous PAM silencing mutations are annotated as such in Tables 1-44. In some of Tables 1-44, RTT are further annotated with a * followed by a number code. As described below, a PE3 or PE3b ngRNA spacers annotated with the same * and number code as an RTT has perfect complementarity to the edit strand post-edit by a PEgRNA containing the RTT. The PBS can be, for example, 5 to 19 nucleotides in length.

The PEgRNA provided in Tables 1-44 can comprise, from 5′ to 3′, the spacer, the gRNA core, the edit template, and the PBS. The 3′ end of the edit template can be contiguous with the 5′ end of the PBS. The PEgRNA can comprise multiple RNA molecules (e.g., a crRNA containing the PEgRNA spacer and a tracrRNA containing the extension arm) or can be a single gRNA molecule. Any PEgRNA exemplified in Tables 1-44 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Tables 1-44 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Tables 1-44 can be used in a Prime Editing system further comprising a nick guide RNA (ngRNA). Such ngRNA can comprise a spacer comprising at its 3′ end a sequence corresponding to nucleotides 4-20 of any ngRNA spacer listed in the same table as the PEgRNA spacer and a gRNA core capable of complexing with a Cas9 protein. For example, the sequence in the spacer of the ngRNA can comprise nucleotides 4-20, 3-20, 2-20, or 1-20 of the listed spacer. In some embodiments, the spacer of the ngRNA is the complete sequence of an ngRNA spacer listed in the same table as the PEgRNA spacer. The ngRNA spacers in Tables 1-44 are annotated with their PAM sequences, enabling selection of an appropriate Cas9 protein. It can be advantageous to select an ngRNA spacer that has a PAM sequence compatible with the Cas9 protein used in the Prime Editor with the PEgRNA, thus avoiding the need to use two different Cas9 proteins. The ngRNA can comprise multiple RNA molecules (e.g., a crRNA containing the ngRNA spacer and a tracrRNA) or can be a single gRNA molecule. The ngRNA is capable of directing a complexed Cas9 protein to bind the edit strand of the USH2A gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b spacer annotated with a * followed by a number code has perfect complementarity to the edit strand post-edit with a PEgRNA containing an RTT from the same Table and annotated with the same number code.

Any ngRNA sequence provided in Tables 1-44 may comprise, or further comprise, a 3′ motif at their 3′ end, for example, a series of 1, 2, 3, 4, 5, 6, 7 or more U nucleotides. In some embodiments, the ngRNA comprises 4 U nucleotides at its 3′ end. Without being bound by theory, such 3′ motifs are believed to increase ngRNA stability. The ngRNA may alternatively or additionally comprise one or more chemical modifications, such as phosphorothioate (PS) bond(s), 2′-O-methylated (2′-Ome) nucleotides, or a combination thereof. In some embodiments, the ngRNA comprise 3′ mN*mN*mN*N and 5′mN*mN*mN* modifications, where m indicates that the nucleotide contains a 2′-O-Me modification and a * indicates the presence of a phosphorothioate bond. NgRNA sequences may alternatively be adapted for expression from a DNA template, for example, by including a 5′ terminal G if the spacer of the ngRNA begins with another nucleotide, by including 6 or 7 U nucleotides at the 3′ end of the ngRNA, or both.

In some embodiments, the gRNA core for the PEgRNA and/or the ngRNA comprises a sequence selected from SEQ ID NOs: 7543, 7544, 7545, or 7546. In some embodiments, the gRNA core comprises SEQ ID NO: 7544.

Table 1 provides Prime Editing guide RNAs (PEgRNAs) that can be used with any Prime Editor containing a Cas9 protein capable of recognizing a TAG PAM sequence. The PEgRNAs of Table 1 can also be used in Prime Editing systems further comprising a nick guide RNA (ngRNA). Such PEgRNAs and Prime Editing systems can be used, for example, to correct an c.2299delG mutation in USH2a.

The PEgRNAs exemplified in Table 1 comprise: (a) a spacer comprising at its 3′ end a sequence corresponding to sequence number 618; (b) a gRNA core capable of complexing with a Cas9 protein, and (c) an extension arm comprising: (i) an editing template at least 9 nucleotides in length and comprising at its 3′ end a sequence corresponding to sequence number 635, and (ii) a prime binding site (PBS) comprising at its 5′ end a sequence corresponding to sequence number 621. The PEgRNA spacer can be, for example, 17-22 nucleotides in length and can comprise the sequence corresponding to any one of sequence numbers 618, 160, 402, 32, 619, or 620. In some embodiments, the PEgRNA spacer comprises sequence number 32. The PEgRNA spacers in Table 1 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, or 666. The PBS can be, for example, 5 to 19 nucleotides in length and can comprise the sequence corresponding to any one of sequence numbers 621, 622, 623, 624, 625, 626, 627, 628, 629, 630, 631, 632, 633, 142, or 634.

Any of the PEgRNAs of Table 1 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 1 and a gRNA core capable of complexing with a Cas9 protein. For example, the sequence in the spacer of the ngRNA can comprise nucleotides 4-20, 3-20, 2-20, or 1-20 of sequence number 667, 668, or 669. In some embodiments, the spacer of the ngRNA is a ngRNA spacer listed in Table 1. The ngRNA spacers in Table 1 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins.

Table 2 provides Prime Editing guide RNAs (PEgRNAs) that can be used with any Prime Editor containing a Cas9 protein capable of recognizing an ACAAAT PAM sequence. The PEgRNAs of Table 2 can also be used in Prime Editing systems further comprising a nick guide RNA (ngRNA). Such PEgRNAs and Prime Editing systems can be used, for example, to correct a c.2299delG mutation in USH2a. Some PEgRNAs in Table 2 can also be used to correct a c.2276 G->T mutation in USH2a.

The PEgRNAs exemplified in Table 2 comprise: (a) a spacer comprising at its 3′ end a sequence corresponding to sequence number 618; (b) a gRNA core capable of complexing with a Cas9 protein, and (c) an extension arm comprising: (i) an editing template at least 40 nucleotides in length and comprising at its 3′ end a sequence corresponding to sequence number 512, and (ii) a prime binding site (PBS) comprising at its 5′ end a sequence corresponding to sequence number 474. The PEgRNA spacer can be, for example, 17-22 nucleotides in length and can comprise the sequence corresponding to any one of sequence numbers 469, 470, 471, 288, 472, or 473. In some embodiments, the PEgRNA spacer comprises sequence number 288. The PEgRNA spacers in Table 2 are annotated with their PAM sequence(s), enabling the selection of an appropriate Cas9 protein. The editing template can be referred to as a reverse transcription template (RTT). The editing template can encode wildtype USH2a gene sequence. The PBS can be, for example, 5 to 19 nucleotides in length and can comprise the sequence corresponding to any one of sequence numbers 474, 475, 476, 477, 478, 479, 480, 481, 482, 483, 484, 485, 486, 487, or 488. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 2 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 2 and a gRNA core capable of complexing with a Cas9 protein. For example, the sequence in the spacer of the ngRNA can comprise nucleotides 4-20, 3-20, 2-20, or 1-20 of sequence number 376, 144, 378, 379, 380, or 381. In some embodiments, the spacer of the ngRNA is a ngRNA spacer listed in Table 2. The ngRNA spacers in Table 2 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins.

Table 3 provides Prime Editing guide RNAs (PEgRNAs) that can be used with any Prime Editor containing a Cas9 protein capable of recognizing a TG PAM sequence. The PEgRNAs of Table 3 can also be used in Prime Editing systems further comprising a nick guide RNA (ngRNA). Such PEgRNAs and Prime Editing systems can be used, for example, to correct a c.2299delG mutation in USH2a. Some PEgRNAs in Table 3 can also be used to correct a c.2276 G->T mutation in USH2a.

The PEgRNAs exemplified in Table 3 comprise: (a) a spacer comprising at its 3′ end a sequence corresponding to sequence number 1; (b) a gRNA core capable of complexing with a Cas9 protein, and (c) an extension arm comprising: (i) an editing template at least 11 nucleotides in length and comprising at its 3′ end a sequence corresponding to any one of sequence numbers 670, 671, 672, 673, or 674, and (ii) a prime binding site (PBS) comprising at its 5′ end a sequence corresponding to sequence number 7. The PEgRNA spacer can be, for example, 17-22 nucleotides in length and can comprise the sequence corresponding to any one of sequence numbers 1-6. In some embodiments, the PEgRNA spacer comprises sequence number 4. The PEgRNA spacers in Table 3 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 670, 671, 672, 673, 675, 677, 679, 681, 683, 685, 687, 689, 691, 693, 695, 697, 699, 701, 703, 705, 707, 709, 711, 713, 715, 717, 22, 24, 26, or 28. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 674, 676, 678, 680, 682, 684, 686, 688, 690, 692, 694, 696, 698, 700, 702, 704, 706, 708, 710, 712, 714, 716, 718, 23, 25, 27, or 29. The PBS can be, for example, 5 to 19 nucleotides in length and can comprise the sequence corresponding to any one of sequence numbers 7, 8, 9, 10, 11, 12, 13, 14, 15, 16, 17, 18, 19, 20, or 21. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 3 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 3 and a gRNA core capable of complexing with a Cas9 protein. For example, the sequence in the spacer of the ngRNA can comprise nucleotides 4-20, 3-20, 2-20, or 1-20 of sequence number 30, 31, 33, 34, 35, 282, 36, 37, 38, 39, 40, 41, 42, 43, 44, 45, 46, 47, 48, 49, 50, 51, 52, 53, or 54. In some embodiments, the spacer of the ngRNA is a ngRNA spacer listed in Table 3. The ngRNA spacers in Table 3 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 3 annotated with the same * and number code as an RTT in Table 3 has perfect complementarity to the edit strand post-edit by a PEgRNA containing the RTT.

Table 4 provides Prime Editing guide RNAs (PEgRNAs) that can be used with any Prime Editor containing a Cas9 protein capable of recognizing a TTGG PAM sequence. The PEgRNAs of Table 4 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 4 comprise: (a) a spacer comprising at its 3′ end a sequence corresponding to sequence number 1556; (b) a gRNA core capable of complexing with a Cas9 protein, and (c) an extension arm comprising: (i) an editing template at least 24 nucleotides in length and comprising at its 3′ end a sequence corresponding to any one of sequence numbers 1561, 1562, 1563, 1564, or 1565, and (ii) a prime binding site (PBS) comprising at its 5′ end a sequence corresponding to sequence number 1541. The PEgRNA spacer can be, for example, 17-22 nucleotides in length and can comprise the sequence corresponding to any one of sequence numbers 1556, 1557, 1558, 1079, 1559, or 1560. In some embodiments, the PEgRNA spacer comprises sequence number 1079. The PEgRNA spacers in Table 4 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1561, 1562, 1563, 1564, or 1565. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 1564, 1566, 1567, 1568, 1570, 1571, 1572, 1574, 1575, 1576, 1578, 1579, 1580, 1582, 1583, 1584, 1586, 1587, 1588, 1590, 1591, 1592, 1594, 1595, 1596, 1598, 1599, 1600, 1602, 1603, 1604, 1606, 1607, 1608, 1610, 1611, 1612, 1614, 1615, 1616, 1618, or 1619. The PBS can be, for example, 5 to 19 nucleotides in length and can comprise the sequence corresponding to any one of sequence numbers 1541, 1542, 1543, 1544, 1545, 1546, 1547, 1548, 1549, 1550, 1551, 1552, 1553, 1554, or 1555.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 4 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 4 and a gRNA core capable of complexing with a Cas9 protein. For example, the sequence in the spacer of the ngRNA can comprise nucleotides 4-20, 3-20, 2-20, or 1-20 of sequence number 30, 962, 1043, 290, 1620, 1621, 1622, or 1623. In some embodiments, the spacer of the ngRNA is a ngRNA spacer listed in Table 4. The ngRNA spacers in Table 4 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 4 annotated with the same * and number code as an RTT in Table 4 has perfect complementarity to the edit strand post-edit by a PEgRNA containing the RTT.

Table 5 provides Prime Editing guide RNAs (PEgRNAs) that can be used with any Prime Editor containing a Cas9 protein capable of recognizing a CTGG PAM sequence. The PEgRNAs of Table 5 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 5 comprise: (a) a spacer comprising at its 3′ end a sequence corresponding to sequence number 950; (b) a gRNA core capable of complexing with a Cas9 protein, and (c) an extension arm comprising: (i) an editing template at least 16 nucleotides in length and comprising at its 3′ end a sequence corresponding to sequence number 1638, and (ii) a prime binding site (PBS) comprising at its 5′ end a sequence corresponding to sequence number 1624. The PEgRNA spacer can be, for example, 17-22 nucleotides in length and can comprise the sequence corresponding to any one of sequence numbers 950, 954, 958, 962, 966, or 970. In some embodiments, the PEgRNA spacer comprises sequence number 962. The PEgRNA spacers in Table 5 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1638, 932, 933, 934, 379, 935, 936, 1639, 1640, 1641, 1642, 1643, 1644, 1645, 1646, 1647, 1648, 1649, 1650, 1651, 1652, 1653, 1654, 1655, or 1656. The PBS can be, for example, 5 to 19 nucleotides in length and can comprise the sequence corresponding to any one of sequence numbers 1624, 1625, 1626, 1627, 1628, 1629, 1630, 1631, 320, 1632, 1633, 1634, 1635, 1636, or 1637. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 5 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 5 and a gRNA core capable of complexing with a Cas9 protein. For example, the sequence in the spacer of the ngRNA can comprise nucleotides 4-20, 3-20, 2-20, or 1-20 of sequence number 667, 1079, 1657, or 379. In some embodiments, the spacer of the ngRNA is a ngRNA spacer listed in Table 5. The ngRNA spacers in Table 5 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 6 provides Prime Editing guide RNAs (PEgRNAs) that can be used with any Prime Editor containing a Cas9 protein capable of recognizing an AG PAM sequence. The PEgRNAs of Table 6 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 6 comprise: (a) a spacer comprising at its 3′ end a sequence corresponding to sequence number 1673; (b) a gRNA core capable of complexing with a Cas9 protein, and (c) an extension arm comprising: (i) an editing template at least 30 nucleotides in length and comprising at its 3′ end a sequence corresponding to any one of sequence numbers 1679, 1680, 1681, 1682, 1683, or 1684, and (ii) a prime binding site (PBS) comprising at its 5′ end a sequence corresponding to sequence number 1658. The PEgRNA spacer can be, for example, 17-22 nucleotides in length and can comprise the sequence corresponding to any one of sequence numbers 1673, 1674, 1675, 1676, 1677, or 1678. In some embodiments, the PEgRNA spacer comprises sequence number 1676. The PEgRNA spacers in Table 6 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1679, 1680, 1681, 1682, 1685, 1688, 1691, 1694, 1697, 1700, 1703, 1706, or 1706. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 1683, 1684, 1686, 1687, 1689, 1690, 1692, 1693, 1695, 1696, 1698, 1699, 1701, 1702, 1704, or 1705. The PBS can be, for example, 5 to 19 nucleotides in length and can comprise the sequence corresponding to any one of sequence numbers 1658, 1659, 1660, 1661, 1662, 1663, 1664, 1665, 1666, 1667, 1668, 1669, 1670, 1671, or 1672. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 6 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 6 and a gRNA core capable of complexing with a Cas9 protein. For example, the sequence in the spacer of the ngRNA can comprise nucleotides 4-20, 3-20, 2-20, or 1-20 of sequence number 1706, 1707, 1708, 30, 1709, 1710, 1711, 33, 34, 1712, 35, 282, 36, 37, 1713, 38, 39, 40, 42, 43, 1714, 1715, 1716, 44, 1717, 45, 46, 48, 50, 51, or 52. In some embodiments, the spacer of the ngRNA is a ngRNA spacer listed in Table 6. The ngRNA spacers in Table 6 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 7 provides Prime Editing guide RNAs (PEgRNAs) that can be used with any Prime Editor containing a Cas9 protein capable of recognizing a GG PAM sequence. The PEgRNAs of Table 7 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 7 comprise: (a) a spacer comprising at its 3′ end a sequence corresponding to sequence number 1733; (b) a gRNA core capable of complexing with a Cas9 protein, and (c) an extension arm comprising: (i) an editing template at least 22 nucleotides in length and comprising at its 3′ end a sequence corresponding to any one of sequence numbers 1739, and (ii) a prime binding site (PBS) comprising at its 5′ end a sequence corresponding to sequence number 1718. The PEgRNA spacer can be, for example, 17-22 nucleotides in length and can comprise the sequence corresponding to any one of sequence numbers 1733, 1734, 1735, 1736, 1737, or 1738. In some embodiments, the PEgRNA spacer comprises sequence number 1736. The PEgRNA spacers in Table 7 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1739, 1740, 1741, 1742, 1743, 1744, 1745, 1746, 1747, 1748, 1749, 1750, 1751, 1752, 1753, 1754, 1755, 1756, or 1757. The PBS can be, for example, 5 to 19 nucleotides in length and can comprise the sequence corresponding to any one of sequence numbers 1718, 1719, 1720, 1721, 1722, 1723, 1724, 1725, 1726, 1727, 1728, 1729, 1730, 1731, or 1732. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 7 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 7 and a gRNA core capable of complexing with a Cas9 protein. For example, the sequence in the spacer of the ngRNA can comprise nucleotides 4-20, 3-20, 2-20, or 1-20 of sequence number 30, 33, 34, 1712, 35, 282, 36, 37, 1713, 38, 39, 40, 41, 42, 43, 44, 1717, 45, 46, 48, 49, 50, 51, 52, or 54. In some embodiments, the spacer of the ngRNA is a ngRNA spacer listed in Table 7. The ngRNA spacers in Table 7 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 8 provides Prime Editing guide RNAs (PEgRNAs) that can be used with any Prime Editor containing a Cas9 protein capable of recognizing an AAG PAM sequence. The PEgRNAs of Table 8 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 8 comprise: (a) a spacer comprising at its 3′ end a sequence corresponding to sequence number 1773; (b) a gRNA core capable of complexing with a Cas9 protein, and (c) an extension arm comprising: (i) an editing template at least 31 nucleotides in length and comprising at its 3′ end a sequence corresponding to any one of sequence numbers 1778, 1779, or 1785 and (ii) a prime binding site (PBS) comprising at its 5′ end a sequence corresponding to sequence number 1758. The PEgRNA spacer can be, for example, 17-22 nucleotides in length and can comprise the sequence corresponding to any one of sequence numbers 1773, 1774, 1775, 668, 1776, or 1777. In some embodiments, the PEgRNA spacer comprises sequence number 668. The PEgRNA spacers in Table 8 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1778, 1780, 1782, 1784, 1787, 1790, 1793, 1796, 1799, or 1802. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 1779, 1781, 1783, 1785, 1786, 1788, 1789, 1791, 1792, 1794, 1795, 1797, 1798, 1800, 1801, 1803, or 1804. The PBS can be, for example, 5 to 19 nucleotides in length and can comprise the sequence corresponding to any one of sequence numbers 1758, 1759, 1760, 1761, 1762, 1763, 1764, 1765, 1766, 1767, 1768, 1769, 1770, 1771, or 1772. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 8 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 8 and a gRNA core capable of complexing with a Cas9 protein. For example, the sequence in the spacer of the ngRNA can comprise nucleotides 4-20, 3-20, 2-20, or 1-20 of sequence number 1805, 1806, 1807, 32, 284, 285, 1808, 287, 1303, 1621, or 685. In some embodiments, the spacer of the ngRNA is a ngRNA spacer listed in Table 8. The ngRNA spacers in Table 8 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 9 provides Prime Editing guide RNAs (PEgRNAs) that can be used with any Prime Editor containing a Cas9 protein capable of recognizing a CAG PAM sequence. The PEgRNAs of Table 9 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 9 comprise: (a) a spacer comprising at its 3′ end a sequence corresponding to sequence number 1823; (b) a gRNA core capable of complexing with a Cas9 protein, and (c) an extension arm comprising: (i) an editing template at least 11 nucleotides in length and comprising at its 3′ end a sequence corresponding to any one of sequence numbers 1824 or 1825, and (ii) a prime binding site (PBS) comprising at its 5′ end a sequence corresponding to sequence number 1809. The PEgRNA spacer can be, for example, 17-22 nucleotides in length and can comprise the sequence corresponding to any one of sequence numbers 1823, 1327, 1328, 1303, 1331, 1334. In some embodiments, the PEgRNA spacer comprises sequence number 1303. The PEgRNA spacers in Table 9 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1824, 1826, 1828, 1830, 1832, 1834, 1321, 1322, 1323, 1324, 1325, 1326, 1842, 1844, 1846, 1848, 1850, 1852, 1854, 1856, 1858, 1860, 1862, 1864, 1866, 1868, 1870, 1872, 1874, or 1876. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 1825, 1827, 1829, 1831, 1833, 1835, 1836, 1837, 1838, 1839, 1840, 1841, 1843, 1845, 1847, 1849, 1851, 1853, 1855, 1857, 1859, 1861, 1863, 1865, 1867, 1869, 1871, 1873, 1875, or 1877. The PBS can be, for example, 5 to 19 nucleotides in length and can comprise the sequence corresponding to any one of sequence numbers 1809, 1810, 1811, 1812, 1813, 1814, 1815, 1816, 1817, 1818, 1819, 1820, 1821, 330, or 1822. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 9 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 9 and a gRNA core capable of complexing with a Cas9 protein. For example, the sequence in the spacer of the ngRNA can comprise nucleotides 4-20, 3-20, 2-20, or 1-20 of sequence number 667, 668, 669, 747, 1878, 381, or 1879. In some embodiments, the spacer of the ngRNA is a ngRNA spacer listed in Table 9. The ngRNA spacers in Table 9 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 9 annotated with the same * and number code as an RTT in Table 9 has perfect complementarity to the edit strand post-edit by a PEgRNA containing the RTT.

Table 10 provides Prime Editing guide RNAs (PEgRNAs) that can be used with any Prime Editor containing a Cas9 protein capable of recognizing a TG, TGA, or TGAAGT PAM sequence. The PEgRNAs of Table 10 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 10 comprise: (a) a spacer comprising at its 3′ end a sequence corresponding to sequence number 3102; (b) a gRNA core capable of complexing with a Cas9 protein, and (c) an extension arm comprising: (i) an editing template at least 36 nucleotides in length and comprising at its 3′ end a sequence corresponding to any one of sequence numbers 3107, 3108, 3109, 3110, 3111, 3112, 3113, 3114, 3115, 3116, 3117, or 3118, and (ii) a prime binding site (PBS) comprising at its 5′ end a sequence corresponding to sequence number 3087. The PEgRNA spacer can be, for example, 17-22 nucleotides in length and can comprise the sequence corresponding to any one of sequence numbers 3102, 3103, 3104, 380, 3105, or 3106. In some embodiments, the PEgRNA spacer comprises sequence number 380. The PEgRNA spacers in Table 10 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 3107, 3108, 3109, 3113, 3122, 3131, 3140, or 3149. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 3110, 3111, 3112, 3114, 3115, 3116, 3117, 3118, 3119, 3120, 3121, 3123, 3124, 3125, 3126, 3127, 3128, 3129, 3130, 3132, 3133, 3134, 3135, 3136, 3137, 3138, 3139, 3141, 3142, 3143, 3144, 3145, 3146, 3147, 3148, 3150, 3151, 3152, 3153, or 3154. The PBS can be, for example, 5 to 19 nucleotides in length and can comprise the sequence corresponding to any one of sequence numbers 3087, 3088, 3089, 3090, 3091, 3092, 3093, 3094, 3095, 3096, 3097, 3098, 3099, 3100, or 3101.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 10 can comprise a sequence corresponding to any one of sequence numbers 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, or 3545. Any PEgRNA exemplified in Table 10 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 10 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 10 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 10 and a gRNA core capable of complexing with a Cas9 protein. For example, the sequence in the spacer of the ngRNA can comprise nucleotides 4-20, 3-20, 2-20, or 1-20 of sequence number 1706, 30, 1709, 33, 1712, 35, 282, 36, 3155, 3156, 3157, 3158, 37, 1713, 38, 39, 40, 42, 43, 3159, 3160, 3161, 1714, 1715, 1716, 1717, 45, 46, 48, 288, 50, 292, 293, 51, or 52. In some embodiments, the spacer of the ngRNA is a ngRNA spacer listed in Table 10. The ngRNA spacers in Table 10 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 10 annotated with the same * and number code as an RTT in Table 10 has perfect complementarity to the edit strand post-edit by a PEgRNA containing the RTT.

Table 11 provides Prime Editing guide RNAs (PEgRNAs) that can be used with any Prime Editor containing a Cas9 protein capable of recognizing a TG or TGG PAM sequence. The PEgRNAs of Table 11 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 11 comprise: (a) a spacer comprising at its 3′ end a sequence corresponding to sequence number 3561; (b) a gRNA core capable of complexing with a Cas9 protein, and (c) an extension arm comprising: (i) an editing template at least 23 nucleotides in length and comprising at its 3′ end a sequence corresponding to any one of sequence numbers 3567, 3568, 3573, or 3575, and (ii) a prime binding site (PBS) comprising at its 5′ end a sequence corresponding to sequence number 3546. The PEgRNA spacer can be, for example, 17-22 nucleotides in length and can comprise the sequence corresponding to any one of sequence numbers 3561, 3562, 3563, 3564, 3565, or 3566. In some embodiments, the PEgRNA spacer comprises sequence number 3564. The PEgRNA spacers in Table 11 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 3567, 3569, 3571, 3574, 3578, 3582, 3586, 3590, 3594, 3598, 3602, 3606, 3610, 3614, 3618, 3622, 3626, or 3630. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 3570, 3568, 3572, 3573, 3575, 3576, 3577, 3579, 3580, 3581, 3583, 3584, 3585, 3587, 3588, 3589, 3591, 3592, 3593, 3595, 3596, 3597, 3599, 3600, 3601, 3603, 3604, 3605, 3607, 3608, 3609, 3611, 3612, 3613, 3615, 3616, 3617, 3619, 3620, 3621, 3623, 3624, 3625, 3627, 3628, 3629, 3631, or 3632. The PBS can be, for example, 5 to 19 nucleotides in length and can comprise the sequence corresponding to any one of sequence numbers 3546, 3547, 3548, 3549, 3550, 3551, 3552, 3553, 3554, 3555, 3556, 3557, 3558, 3559, or 3560.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 11 can comprise a sequence corresponding to any one of sequence numbers 3639 to 4480. Any PEgRNA exemplified in Table 11 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 11 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 11 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 11 and a gRNA core capable of complexing with a Cas9 protein. For example, the sequence in the spacer of the ngRNA can comprise nucleotides 4-20, 3-20, 2-20, or 1-20 of sequence number 30, 33, 34, 3633, 1712, 3634, 3635, 35, 282, 36, 37, 1713, 38, 39, 40, 41, 42, 43, 44, 3636, 1717, 3637, 3638, 45, 46, 48, 49, 50, 51, 52, or 54. In some embodiments, the spacer of the ngRNA is a ngRNA spacer listed in Table 11. The ngRNA spacers in Table 11 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 11 annotated with the same * and number code as an RTT in Table 11 has perfect complementarity to the edit strand post-edit by a PEgRNA containing the RTT.

Table 12 provides Prime Editing guide RNAs (PEgRNAs) that can be used with any Prime Editor containing a Cas9 protein capable of recognizing a TG or TGTAGT PAM sequence. The PEgRNAs of Table 12 can also be used in Prime Editing systems further comprising a nick guide RNA (ngRNA). Such PEgRNAs and Prime Editing systems can be used, for example, to correct a c.2299delG mutation in USH2a. Some PEgRNAs in Table 12 can also be used to correct a c.2276 G->T mutation in USH2a.

The PEgRNAs exemplified in Table 12 comprise: (a) a spacer comprising at its 3′ end a sequence corresponding to sequence number 400; (b) a gRNA core capable of complexing with a Cas9 protein, and (c) an extension arm comprising: (i) an editing template at least 10 nucleotides in length and comprising at its 3′ end a sequence corresponding to sequence number 5936, and (ii) a prime binding site (PBS) comprising at its 5′ end a sequence corresponding to sequence number 5917. The PEgRNA spacer can be, for example, 17-22 nucleotides in length and can comprise the sequence corresponding to any one of sequence numbers 400, 5932, 5933, 45, 5934, 5935. In some embodiments, the PEgRNA spacer comprises sequence number 45. The PEgRNA spacers in Table 12 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 5936, 5937, 5938, 5939, 5940, 5941, 5942, 5943, 5944, 5945, 5946, 5947, 5948, 5949, 5950, 5951, 5952, 5953, 5954, 5955, 5956, 5957, 5958, 5959, 5960, 5961, 5962, 5963, 5964, 5965, or 5966. The PBS can be, for example, 5 to 19 nucleotides in length and can comprise the sequence corresponding to any one of sequence numbers 5917, 5918, 5919, 5920, 5921, 5922, 5923, 5924, 5925, 5926, 5927, 5928, 5929, 5930, or 5931. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 12 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 12 and a gRNA core capable of complexing with a Cas9 protein. For example, the sequence in the spacer of the ngRNA can comprise nucleotides 4-20, 3-20, 2-20, or 1-20 of sequence number 376, 4, 5971, 4510, 378, 5972, 4634, 4635, 1676, 646, 4639, 3564, 5973, 1736, 4640, 5974, 380, 1324, 381, or 5975. In some embodiments, the spacer of the ngRNA is a ngRNA spacer listed in Table 12. The ngRNA spacers in Table 12 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 12 can comprise a sequence corresponding to sequence number 5967, 4632, 4505, 5968, 5969, 4633, 4507, or 5970.

Table 13 provides Prime Editing guide RNAs (PEgRNAs) that can be used with any Prime Editor containing a Cas9 protein capable of recognizing a GG or GGG PAM sequence. The PEgRNAs of Table 13 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 13 comprise: (a) a spacer comprising at its 3′ end a sequence corresponding to sequence number 6027; (b) a gRNA core capable of complexing with a Cas9 protein, and (c) an extension arm comprising: (i) an editing template at least 14 nucleotides in length and comprising at its 3′ end a sequence corresponding to any one of sequence numbers 6032, 6033, 6038, or 6041, and (ii) a prime binding site (PBS) comprising at its 5′ end a sequence corresponding to sequence number 6013. The PEgRNA spacer can be, for example, 17-22 nucleotides in length and can comprise the sequence corresponding to any one of sequence numbers 6027, 6028, 6029, 33, 6030, or 6031. In some embodiments, the PEgRNA spacer comprises sequence number 33. The PEgRNA spacers in Table 13 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 6032, 6034, 6036, 6039, 6043, 6047, 6051, 6055, 6059, 6063, 6067, 6071, 6075, 6079, 6083, 6087, 6091, 6099, 6095, 6103, 6107, 6111, 6115, 6119, 6123, 6127, or 6131. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 6033, 6035, 6037, 6038, 6040, 6041, 6042, 6044, 6045, 6046, 6048, 6049, 6050, 6052, 6053, 6054, 6056, 6057, 6058, 6060, 6061, 6062, 6064, 6065, 6066, 6068, 6069, 6070, 6072, 6073, 6074, 6076, 6077, 6078, 6080, 6081, 6082, 6084, 6085, 6086, 6088, 6089, 6090, 6092, 6093, 6094, 6096, 6097, 6098, 6100, 6101, 6102, 6104, 6105, 6106, 6108, 6109, 6110, 6112, 6113, 6114, 6116, 6117, 6118, 6120, 6121, 6122, 6124, 6125, 6126, 6128, 6129, 6130, 6132, or 6133. The PBS can be, for example, 5 to 19 nucleotides in length and can comprise the sequence corresponding to any one of sequence numbers 6013, 6014, 6015, 6016, 6017, 6018, 6019, 6020, 6021, 6022, 324, 6023, 6024, 6025, or 6026.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 13 can comprise a sequence corresponding to any one of sequence numbers 6139 to 7068. Any PEgRNA exemplified in Table 13 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 13 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 13 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 13 and a gRNA core capable of complexing with a Cas9 protein. For example, the sequence in the spacer of the ngRNA can comprise nucleotides 4-20, 3-20, 2-20, or 1-20 of sequence number 4, 5971, 4510, 6136, 2008, 6137, 6138, 5972, 4634, 4635, 1676, 646, 4639, 3564, 5973, 1736, 4640, 2090, 380, 1098, 1324, or 5975. In some embodiments, the spacer of the ngRNA is a ngRNA spacer listed in Table 13. The ngRNA spacers in Table 13 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 13 annotated with the same * and number code as an RTT in Table 13 has perfect complementarity to the edit strand post-edit by a PEgRNA containing the RTT. Exemplary ngRNA provided in Table 13 can comprise a sequence corresponding to sequence number 5967, 4504, 6134, 4632, 4505, 5968, 5969, 4506, 6135, 4633, 4507, or 5970.

Table 14 provides Prime Editing guide RNAs (PEgRNAs) that can be used with any Prime Editor containing a Cas9 protein capable of recognizing a TG or TGCAAT PAM sequence. The PEgRNAs of Table 14 can also be used in Prime Editing systems further comprising a nick guide RNA (ngRNA). Such PEgRNAs and Prime Editing systems can be used, for example, to correct a c.2299delG mutation in USH2a. The PEgRNAs in Table 14 can also be used to correct a c.2276 G->T mutation in USH2a.

The PEgRNAs exemplified in Table 14 comprise: (a) a spacer comprising at its 3′ end a sequence corresponding to sequence number 296; (b) a gRNA core capable of complexing with a Cas9 protein, and (c) an extension arm comprising: (i) an editing template at least 32 nucleotides in length and comprising at its 3′ end a sequence corresponding to any one of sequence numbers 358 or 359, and (ii) a prime binding site (PBS) comprising at its 5′ end a sequence corresponding to sequence number 301. The PEgRNA spacer can be, for example, 17-22 nucleotides in length and can comprise the sequence corresponding to any one of sequence numbers 296, 297, 298, 282, 299, or 300. In some embodiments, the PEgRNA spacer comprises sequence number 282. The PEgRNA spacers in Table 14 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 358, 360, 362, 364, 366, 368, 370, 372, or 374. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 359, 361, 363, 365, 367, 369, 371, 373, or 375. The PBS can be, for example, 5 to 19 nucleotides in length and can comprise the sequence corresponding to any one of sequence numbers 301, 302, 303, 304, 305, 306, 307, 308, 309, 310, 311, 312, 313, 314, or 315. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 14 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 14 and a gRNA core capable of complexing with a Cas9 protein. For example, the sequence in the spacer of the ngRNA can comprise nucleotides 4-20, 3-20, 2-20, or 1-20 of sequence number 376, 4, 58, 1898, 144, 377, 4509, 4510, 378, 2008, 4634, 4635, 1676, 4519, 646, 379, 4639, 3564, 5973, 1736, 4640, 2090, 380, 1098, 1324, or 381. In some embodiments, the spacer of the ngRNA is a ngRNA spacer listed in Table 14. The ngRNA spacers in Table 14 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 14 annotated with the same * and number code as an RTT in Table 14 has perfect complementarity to the edit strand post-edit by a PEgRNA containing the RTT. Exemplary ngRNA provided in Table 14 can comprise a sequence corresponding to sequence number 4504, 4632, 4505, 4506, 4633, or 4507.

Table 15 provides Prime Editing guide RNAs (PEgRNAs) that can be used with any Prime Editor containing a Cas9 protein capable of recognizing a TG, TGG, or TGGG PAM sequence. The PEgRNAs of Table 15 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 15 comprise: (a) a spacer comprising at its 3′ end a sequence corresponding to sequence number 2099; (b) a gRNA core capable of complexing with a Cas9 protein, and (c) an extension arm comprising: (i) an editing template at least 15, 16, 17, or 18 nucleotides in length and comprising at its 3′ end a sequence corresponding to any one of sequence numbers 7108, 7110, 7111, or 7112, and (ii) a prime binding site (PBS) comprising at its 5′ end a sequence corresponding to sequence number 7094. The PEgRNA spacer can be, for example, 17-22 nucleotides in length and can comprise the sequence corresponding to any one of sequence numbers 2099, 2103, 2107, 30, 2114, or 2118. In some embodiments, the PEgRNA spacer comprises sequence number 30. The PEgRNA spacers in Table 15 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 7108, 7109, 2087, 2088, 2089, 2090, 2091, 2092, 7126, 7130, 7134, 7138, 7142, 7146, 7150, 7154, 7158, 7162, 7166, 7170, 7174, 7178, 7182, 7186, 7190, or 7194. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 7110, 7111, 7112, 7113, 7114, 7115, 7116, 7117, 7118, 7119, 7120, 7121, 7122, 7123, 7124, 7125, 7127, 7128, 7129, 7131, 7133, 7132, 7135, 7136, 7137, 7139, 7140, 7141, 7143, 7144, 7145, 7147, 7148, 7149, 7151, 7152, 7153, 7155, 7156, 7157, 7159, 7160, 7161, 7163, 7164, 7165, 7167, 7169, 7168, 7171, 7172, 7173, 7175, 7176, 7177, 7179, 7180, 7181, 7183, 7184, 7185, 7187, 7188, 7189, 7191, 7192, 7193, 7195, or 7196. The PBS can be, for example, 5 to 19 nucleotides in length and can comprise the sequence corresponding to any one of sequence numbers 7094, 7095, 7096, 7097, 7098, 7099, 7100, 7101, 7102, 322, 7103, 7104, 7105, 7106, or 7107.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 15 can comprise a sequence corresponding to any one of sequence numbers 7200 to 7225. Any PEgRNA exemplified in Table 15 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 15 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 15 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 15 and a gRNA core capable of complexing with a Cas9 protein. For example, the sequence in the spacer of the ngRNA can comprise nucleotides 4-20, 3-20, 2-20, or 1-20 of sequence number 4, 5971, 667, 1898, 1079, 4510, 1657, 2008, 5972, 4634, 4635, 1676, 646, 7197, 379, 7198, 7199, 4639, 3564, 5973, 1736, 4640, 2090, 380, 1098, 1324, or 5975. In some embodiments, the spacer of the ngRNA is a ngRNA spacer listed in Table 15. The ngRNA spacers in Table 15 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 15 annotated with the same * and number code as an RTT in Table 15 has perfect complementarity to the edit strand post-edit by a PEgRNA containing the RTT. Exemplary ngRNA provided in Table 15 can comprise a sequence corresponding to sequence number 5967, 4504, 4632, 4505, 5968, 5969, 4506, 4633, 4507, or 5970.

Table 16 provides Prime Editing guide RNAs (PEgRNAs) that can be used with any Prime Editor containing a Cas9 protein capable of recognizing a TG PAM sequence. The PEgRNAs of Table 16 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 16 comprise: (a) a spacer comprising at its 3′ end a sequence corresponding to sequence number 7240; (b) a gRNA core capable of complexing with a Cas9 protein, and (c) an extension arm comprising: (i) an editing template at least 6 nucleotides in length and comprising at its 3′ end a sequence corresponding to any one of sequence numbers 7246, and (ii) a prime binding site (PBS) comprising at its 5′ end a sequence corresponding to sequence number 7226. The PEgRNA spacer can be, for example, 17-22 nucleotides in length and can comprise the sequence corresponding to any one of sequence numbers 7240, 7241, 7242, 7243, 7244, or 7245. In some embodiments, the PEgRNA spacer comprises sequence number 7243. The PEgRNA spacers in Table 16 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 7246 to 7280. The PBS can be, for example, 5 to 19 nucleotides in length and can comprise the sequence corresponding to any one of sequence numbers 7226, 7227, 7228, 7229, 7230, 7231, 7232, 7233, 7234, 7235, 7236, 7237, 7238, 7239, or 57. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 16 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 16 and a gRNA core capable of complexing with a Cas9 protein. For example, the sequence in the spacer of the ngRNA can comprise nucleotides 4-20, 3-20, 2-20, or 1-20 of sequence number 4, 5971, 4510, 5972, 4634, 4635, 1676, 646, 4639, 3564, 5973, 1736, 4640, 380, or 5975. In some embodiments, the spacer of the ngRNA is a ngRNA spacer listed in Table 16. The ngRNA spacers in Table 16 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. Exemplary ngRNA provided in Table 16 can comprise a sequence corresponding to sequence number 5967, 4632, 4505, 5968, 5969, 4633, 4507, or 5970.

Table 17 provides Prime Editing guide RNAs (PEgRNAs) that can be used with any Prime Editor containing a Cas9 protein capable of recognizing a GG or GGCAGT PAM sequence. The PEgRNAs of Table 17 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 17 comprise: (a) a spacer comprising at its 3′ end a sequence corresponding to sequence number 1102; (b) a gRNA core capable of complexing with a Cas9 protein, and (c) an extension arm comprising: (i) an editing template at least 13 nucleotides in length and comprising at its 3′ end a sequence corresponding to any one of sequence numbers 7295, 7298, 7300, or 7301, and (ii) a prime binding site (PBS) comprising at its 5′ end a sequence corresponding to sequence number 7281. The PEgRNA spacer can be, for example, 17-22 nucleotides in length and can comprise the sequence corresponding to any one of sequence numbers 1102, 1103, 1104, 36, 1107, or 1109. In some embodiments, the PEgRNA spacer comprises sequence number 36. The PEgRNA spacers in Table 17 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 7295, 7296, 7297, 7299, 1095, 1096, 1097, 1098, 1099, 1100, 7321, 7325, 7329, 7333, 7337, 7341, 7345, 7349, 7353, 7357, 7361, 7365, 7369, 7373, 7377, 7381, 7385, or 7389. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 7298, 7300, 7301, 7302, 7303, 7304, 7305, 7306, 7307, 7308, 7309, 7310, 7311, 7312, 7313, 7314, 7315, 7316, 7317, 7318, 7319, 7320, 7322, 7323, 7324, 7326, 7327, 7328, 7330, 7331, 7332, 7334, 7335, 7336, 7338, 7339, 7340, 7342, 7343, 7344, 7346, 7347, 7348, 7350, 7351, 7352, 7354, 7355, 7356, 7358, 7359, 7360, 7362, 7363, 7364, 7366, 7367, 7368, 7370, 7371, 7372, 7374, 7375, 7376, 7378, 7379, 7380, 7382, 7383, 7384, 7386, 7387, 7388, 7390, or 7391. The PBS can be, for example, 5 to 19 nucleotides in length and can comprise the sequence corresponding to any one of sequence numbers 7281, 7282, 7283, 7284, 7285, 7286, 7287, 7288, 7289, 7290, 7291, 326, 7292, 7293, or 7294. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 17 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 17 and a gRNA core capable of complexing with a Cas9 protein. For example, the sequence in the spacer of the ngRNA can comprise nucleotides 4-20, 3-20, 2-20, or 1-20 of sequence number 376, 4, 5971, 4510, 378, 5972, 4634, 4635, 1676, 646, 379, 4639, 3564, 5973, 1736, 4640, 5974, 7116, 2090, 7117, 7118, 380, 7311, 1098, 7312, 7313, 7392, 1324, 7393, 7394, 381, or 5975. In some embodiments, the spacer of the ngRNA is a ngRNA spacer listed in Table 17. The ngRNA spacers in Table 17 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 17 annotated with the same * and number code as an RTT in Table 17 has perfect complementarity to the edit strand post-edit by a PEgRNA containing the RTT. Exemplary ngRNA provided in Table 17 can comprise a sequence corresponding to sequence number 5967, 4632, 4505, 5968, 5969, 4633, 4507, or 5970.

Table 18 provides Prime Editing guide RNAs (PEgRNAs) that can be used with any Prime Editor containing a Cas9 protein capable of recognizing an AG PAM sequence. The PEgRNAs of Table 18 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 18 comprise: (a) a spacer comprising at its 3′ end a sequence corresponding to sequence number 73; (b) a gRNA core capable of complexing with a Cas9 protein, and (c) an extension arm comprising: (i) an editing template at least 8 nucleotides in length and comprising at its 3′ end a sequence corresponding to sequence number 7412, and (ii) a prime binding site (PBS) comprising at its 5′ end a sequence corresponding to sequence number 7395. The PEgRNA spacer can be, for example, 17-22 nucleotides in length and can comprise the sequence corresponding to any one of sequence numbers 73, 7408, 161, 7409, 7410, or 7411. In some embodiments, the PEgRNA spacer comprises sequence number 7409. The PEgRNA spacers in Table 18 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 7412, 7413, 7414, 7415, 7416, 7417, 7418, 7419, 7420, 7421, 7422, 7423, 7424, 7425, 7426, 7427, 7428, 7429, 7430, 7431, 7432, 7433, 7434, 7435, 7436, 7437, 7438, 7439, 7440, 7441, 7442, 7443, or 7444. The PBS can be, for example, 5 to 19 nucleotides in length and can comprise the sequence corresponding to any one of sequence numbers 7395, 7396, 7397, 7398, 7399, 7400, 7401, 7402, 7403, 7404, 7405, 7406, 55, 7407, or 143. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 18 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 18 and a gRNA core capable of complexing with a Cas9 protein. For example, the sequence in the spacer of the ngRNA can comprise nucleotides 4-20, 3-20, 2-20, or 1-20 of sequence number 4, 5971, 4510, 5972, 4634, 4635, 1676, 646, 4639, 3564, 5973, 1736, 4640, 380, or 5975. In some embodiments, the spacer of the ngRNA is a ngRNA spacer listed in Table 18. The ngRNA spacers in Table 18 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 18 can comprise a sequence corresponding to sequence number 5967, 4632, 4505, 5968, 5969, 4633, 4507, 5970.

Table 19 provides Prime Editing guide RNAs (PEgRNAs) that can be used with any Prime Editor containing a Cas9 protein capable of recognizing an AG PAM sequence. The PEgRNAs of Table 19 can also be used in Prime Editing systems further comprising a nick guide RNA (ngRNA). Such PEgRNAs and Prime Editing systems can be used, for example, to correct a c.2299delG mutation in USH2a.

The PEgRNAs exemplified in Table 19 comprise: (a) a spacer comprising at its 3′ end a sequence corresponding to sequence number 7459; (b) a gRNA core capable of complexing with a Cas9 protein, and (c) an extension arm comprising: (i) an editing template at least 10 nucleotides in length and comprising at its 3′ end a sequence corresponding to any one of sequence numbers 7461 or 7465, and (ii) a prime binding site (PBS) comprising at its 5′ end a sequence corresponding to sequence number 7445. The PEgRNA spacer can be, for example, 17-22 nucleotides in length and can comprise the sequence corresponding to any one of sequence numbers 7459, 7460, 1418, 42, 1420, or 1426. In some embodiments, the PEgRNA spacer comprises sequence number 42. The PEgRNA spacers in Table 19 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 7461, 7462, 7463, 7464, 7466, 7468, 7470, 1412, 1413, 1414, 381, 1415, 1416, 7477, 7479, 7481, 7483, 7485, 7487, 7489, 7491, 7493, 7495, 7497, 7499, 7501, 7503, 7505, 7507, 7509, or 7511. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 7465, 7467, 7469, 7471, 7472, 7473, 7474, 1879, 7475, 7476, 7478, 7480, 7482, 7484, 7486, 7488, 7490, 7492, 7494, 7496, 7498, 7500, 7502, 7504, 7506, 7508, 7510, or 7512. The PBS can be, for example, 5 to 19 nucleotides in length and can comprise the sequence corresponding to any one of sequence numbers 7445, 7446, 7447, 7448, 7449, 7450, 7451, 7452, 7453, 7454, 7455, 7456, 7457, 7458, or 332. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 19 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 19 and a gRNA core capable of complexing with a Cas9 protein. For example, the sequence in the spacer of the ngRNA can comprise nucleotides 4-20, 3-20, 2-20, or 1-20 of sequence number 4, 5971, 4510, 5972, 4634, 4635, 1676, 646, 7513, 4639, 3564, 5973, 1736, 4640, 380, 1098, 7514, 1324, 1839, or 5975. In some embodiments, the spacer of the ngRNA is a ngRNA spacer listed in Table 19. The ngRNA spacers in Table 19 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 19 annotated with the same * and number code as an RTT in Table 19 has perfect complementarity to the edit strand post-edit by a PEgRNA containing the RTT. Exemplary ngRNA provided in Table 19 can comprise a sequence corresponding to sequence number 5967, 4632, 4505, 5968, 5969, 4633, 4507, or 5970.

Table 20 provides Prime Editing guide RNAs (PEgRNAs) that can be used with any Prime Editor containing a Cas9 protein capable of recognizing a TG PAM sequence. The PEgRNAs of Table 20 can also be used in Prime Editing systems further comprising a nick guide RNA (ngRNA). Such PEgRNAs and Prime Editing systems can be used, for example, to correct a c.2276 G->T mutation in USH2a. The PEgRNAs in Table 20 can also be used to correct a c.2299delG mutation in USH2a.

The PEgRNAs exemplified in Table 20 comprise: (a) a spacer comprising at its 3′ end a sequence corresponding to sequence number 1; (b) a gRNA core capable of complexing with a Cas9 protein, and (c) an extension arm comprising: (i) an editing template at least 37 nucleotides in length and comprising at its 3′ end a sequence corresponding to any one of sequence numbers 22 or 23, and (ii) a prime binding site (PBS) comprising at its 5′ end a sequence corresponding to sequence number 7. The PEgRNA spacer can be, for example, 17-22 nucleotides in length and can comprise the sequence corresponding to any one of sequence numbers 1, 2, 3, 4, 5, or 6. In some embodiments, the PEgRNA spacer comprises sequence number 4. The PEgRNA spacers in Table 20 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 22, 24, 26, or 28. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 23, 25, 27, or 29. The PBS can be, for example, 5 to 19 nucleotides in length and can comprise the sequence corresponding to any one of sequence numbers 7, 8, 9, 10, 11, 12, 13, 14, 15, 16, 17, 18, 19, 20, or 21. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 20 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 20 and a gRNA core capable of complexing with a Cas9 protein. For example, the sequence in the spacer of the ngRNA can comprise nucleotides 4-20, 3-20, 2-20, or 1-20 of sequence number 30, 31, 32, 33, 34, 35, 36, 37, 38, 39, 40, 41, 42, 43, 44, 45, 46, 47, 48, 49, 50, 51, 52, 53, or 54. In some embodiments, the spacer of the ngRNA is a ngRNA spacer listed in Table 20. The ngRNA spacers in Table 20 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 21 provides Prime Editing guide RNAs (PEgRNAs) that can be used with any Prime Editor containing a Cas9 protein capable of recognizing a TG or TGA PAM sequence. The PEgRNAs of Table 21 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 21 comprise: (a) a spacer comprising at its 3′ end a sequence corresponding to sequence number 55; (b) a gRNA core capable of complexing with a Cas9 protein, and (c) an extension arm comprising: (i) an editing template at least 10 nucleotides in length and comprising at its 3′ end a sequence corresponding to any one of sequence numbers 76 or 77, and (ii) a prime binding site (PBS) comprising at its 5′ end a sequence corresponding to sequence number 61. The PEgRNA spacer can be, for example, 17-22 nucleotides in length and can comprise the sequence corresponding to any one of sequence numbers 55, 56, 57, 58, 59, or 60. In some embodiments, the PEgRNA spacer comprises sequence number 58. The PEgRNA spacers in Table 21 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, or 136. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, or 137. The PBS can be, for example, 5 to 19 nucleotides in length and can comprise the sequence corresponding to any one of sequence numbers 61, 62, 63, 64, 65, 66, 67, 68, 69, 70, 71, 72, 73, 74, or 75. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 21 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 21 and a gRNA core capable of complexing with a Cas9 protein. For example, the sequence in the spacer of the ngRNA can comprise nucleotides 4-20, 3-20, 2-20, or 1-20 of sequence number 31, 34, 138, 35, 139, 37, 38, 39, 41, 43, 44, 140, 48, 49, 51, 52, 53, 54. In some embodiments, the spacer of the ngRNA is a ngRNA spacer listed in Table 21. The ngRNA spacers in Table 21 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 22 provides Prime Editing guide RNAs (PEgRNAs) that can be used with any Prime Editor containing a Cas9 protein capable of recognizing an AAG or AAGAAT PAM sequence. The PEgRNAs of Table 22 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 22 comprise: (a) a spacer comprising at its 3′ end a sequence corresponding to sequence number 141; (b) a gRNA core capable of complexing with a Cas9 protein, and (c) an extension arm comprising: (i) an editing template at least 8 nucleotides in length and comprising at its 3′ end a sequence corresponding to any one of sequence numbers 162, 165, 172, or 173, and (ii) a prime binding site (PBS) comprising at its 5′ end a sequence corresponding to sequence number 147. The PEgRNA spacer can be, for example, 17-22 nucleotides in length and can comprise the sequence corresponding to any one of sequence numbers 141, 142, 143, 144, 145, or 146. In some embodiments, the PEgRNA spacer comprises sequence number 144. The PEgRNA spacers in Table 22 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 162, 163, 164, 166, 168, 170, 174, 178, 182, 186, 190, 194, 198, 202, 206, 210, 214, 218, 222, 226, 230, 234, 238, 242, 246, 250, 254, 258, 262, 266, 270, 274, or 278. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 165, 167, 169, 171, 172, 173, 175, 176, 177, 179, 180, 181, 183, 184, 185, 187, 188, 189, 191, 192, 193, 195, 196, 197, 199, 200, 201, 203, 204, 205, 207, 208, 209, 211, 212, 213, 215, 216, 217, 219, 220, 221, 223, 224, 225, 227, 228, 229, 231, 232, 233, 235, 236, 237, 239, 240, 241, 243, 244, 245, 247, 248, 249, 251, 252, 253, 255, 256, 257, 259, 260, 261, 263, 264, 265, 267, 268, 269, 271, 272, 273, 275, 276, 277, 279, 280, or 281. The PBS can be, for example, 5 to 19 nucleotides in length and can comprise the sequence corresponding to any one of sequence numbers 147, 148, 149, 150, 151, 152, 153, 154, 155, 156, 157, 158, 159, 160, or 161. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 22 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 22 and a gRNA core capable of complexing with a Cas9 protein. For example, the sequence in the spacer of the ngRNA can comprise nucleotides 4-20, 3-20, 2-20, or 1-20 of sequence number 282, 283, 284, 285, 286, 287, 288, 289, 290, 291, 292, 293, 294. In some embodiments, the spacer of the ngRNA is a ngRNA spacer listed in Table 22. The ngRNA spacers in Table 22 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 23 provides Prime Editing guide RNAs (PEgRNAs) that can be used with any Prime Editor containing a Cas9 protein capable of recognizing a TTCAAT PAM sequence. The PEgRNAs of Table 23 can also be used in Prime Editing systems further comprising a nick guide RNA (ngRNA). Such PEgRNAs and Prime Editing systems can be used, for example, to correct a c.2276 G->T mutation in USH2a. Some PEgRNAs in Table 23 can also be used to correct a c.2299delG mutation in USH2a.

The PEgRNAs exemplified in Table 23 comprise: (a) a spacer comprising at its 3′ end a sequence corresponding to sequence number 296; (b) a gRNA core capable of complexing with a Cas9 protein, and (c) an extension arm comprising: (i) an editing template at least 9 nucleotides in length and comprising at its 3′ end a sequence corresponding to any one of sequence numbers 316 or 321, and (ii) a prime binding site (PBS) comprising at its 5′ end a sequence corresponding to sequence number 301. The PEgRNA spacer can be, for example, 17-22 nucleotides in length and can comprise the sequence corresponding to any one of sequence numbers 296, 297, 298, 282, 299, or 300. In some embodiments, the PEgRNA spacer comprises sequence number 282. The PEgRNA spacers in Table 23 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 316, 317, 318, 319, 320, 322, 324, 326, 328, 330, 332, 334, 336, 338, 340, 342, 344, 346, 348, 350, 352, 354, 356, 358, 360, 362, 364, 366, 368, 370, 372, or 374. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 321, 323, 325, 327, 329, 331, 333, 335, 337, 339, 341, 343, 345, 347, 349, 351, 353, 355, 357, 359, 361, 363, 365, 367, 369, 371, 373, or 375. The PBS can be, for example, 5 to 19 nucleotides in length and can comprise the sequence corresponding to any one of sequence numbers 301, 302, 303, 304, 305, 306, 307, 308, 309, 310, 311, 312, 313, 314, or 315. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 23 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 23 and a gRNA core capable of complexing with a Cas9 protein. For example, the sequence in the spacer of the ngRNA can comprise nucleotides 4-20, 3-20, 2-20, or 1-20 of sequence number 376, 144, 377, 378, 379, 380, or 381. In some embodiments, the spacer of the ngRNA is a ngRNA spacer listed in Table 23. The ngRNA spacers in Table 23 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 24 provides Prime Editing guide RNAs (PEgRNAs) that can be used with any Prime Editor containing a Cas9 protein capable of recognizing an AG or AGA PAM sequence. The PEgRNAs of Table 24 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 24 comprise: (a) a spacer comprising at its 3′ end a sequence corresponding to sequence number 382; (b) a gRNA core capable of complexing with a Cas9 protein, and (c) an extension arm comprising: (i) an editing template at least 7 nucleotides in length and comprising at its 3′ end a sequence corresponding to any one of sequence numbers 403 or 406, and (ii) a prime binding site (PBS) comprising at its 5′ end a sequence corresponding to sequence number 388. The PEgRNA spacer can be, for example, 17-22 nucleotides in length and can comprise the sequence corresponding to any one of sequence numbers 382, 383, 384, 385, 386, or 387. In some embodiments, the PEgRNA spacer comprises sequence number 385. The PEgRNA spacers in Table 24 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 403, 404, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, or 467. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, or 468. The PBS can be, for example, 5 to 19 nucleotides in length and can comprise the sequence corresponding to any one of sequence numbers 388, 389, 390, 391, 392, 393, 394, 395, 396, 397, 398, 399, 400, 401, or 402. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 24 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 24 and a gRNA core capable of complexing with a Cas9 protein. For example, the sequence in the spacer of the ngRNA can comprise nucleotides 4-20, 3-20, 2-20, or 1-20 of sequence number 31, 34, 138, 35, 139, 37, 38, 39, 41, 43, 44, 140, 48, 49, 51, 52, 53, or 54. In some embodiments, the spacer of the ngRNA is a ngRNA spacer listed in Table 24. The ngRNA spacers in Table 24 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 25 provides Prime Editing guide RNAs (PEgRNAs) that can be used with any Prime Editor containing a Cas9 protein capable of recognizing an ACAAAT PAM sequence. The PEgRNAs of Table 25 can also be used in Prime Editing systems further comprising a nick guide RNA (ngRNA). Such PEgRNAs and Prime Editing systems can be used, for example, to correct a c.2276 G->T mutation in USH2a. Some PEgRNAs in Table 25 can also be used to correct a c.2299delG mutation in USH2a.

The PEgRNAs exemplified in Table 25 comprise: (a) a spacer comprising at its 3′ end a sequence corresponding to sequence number 469; (b) a gRNA core capable of complexing with a Cas9 protein, and (c) an extension arm comprising: (i) an editing template at least 17 nucleotides in length and comprising at its 3′ end a sequence corresponding to sequence number 489, and (ii) a prime binding site (PBS) comprising at its 5′ end a sequence corresponding to sequence number 474. The PEgRNA spacer can be, for example, 17-22 nucleotides in length and can comprise the sequence corresponding to any one of sequence numbers 469, 470, 471, 288, 472, or 473. In some embodiments, the PEgRNA spacer comprises sequence number 288. The PEgRNA spacers in Table 25 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 489, 490, 491, 492, 493, 494, 495, 496, 497, 498, 499, 500, 501, 502, 503, 504, 505, 506, 507, 508, 509, 510, 511, or 512. The PBS can be, for example, 5 to 19 nucleotides in length and can comprise the sequence corresponding to any one of sequence numbers 474, 475, 476, 477, 478, 479, 480, 481, 482, 483, 484, 485, 486, 487, or 488. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 25 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 25 and a gRNA core capable of complexing with a Cas9 protein. For example, the sequence in the spacer of the ngRNA can comprise nucleotides 4-20, 3-20, 2-20, or 1-20 of sequence number 376, 144, 378, 379, 380, or 381. In some embodiments, the spacer of the ngRNA is a ngRNA spacer listed in Table 25. The ngRNA spacers in Table 25 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 26 provides Prime Editing guide RNAs (PEgRNAs) that can be used with any Prime Editor containing a Cas9 protein capable of recognizing a CTCAGT PAM sequence. The PEgRNAs of Table 26 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 26 comprise: (a) a spacer comprising at its 3′ end a sequence corresponding to sequence number 513; (b) a gRNA core capable of complexing with a Cas9 protein, and (c) an extension arm comprising: (i) an editing template at least 25 nucleotides in length and comprising at its 3′ end a sequence corresponding to any one of sequence numbers 533, 534, 535, 536, or 537, and (ii) a prime binding site (PBS) comprising at its 5′ end a sequence corresponding to sequence number 518. The PEgRNA spacer can be, for example, 17-22 nucleotides in length and can comprise the sequence corresponding to any one of sequence numbers 513, 514, 515, 293, 516, or 517. In some embodiments, the PEgRNA spacer comprises sequence number 293. The PEgRNA spacers in Table 26 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 537, 542, 547, 552, 557, 562, 567, 572, 577, 582, 587, 592, 597, 602, 607, or 612. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 533, 534, 535, 536, 538, 539, 540, 541, 543, 544, 545, 546, 548, 549, 550, 551, 553, 554, 555, 556, 558, 559, 560, 561, 563, 564, 565, 566, 568, 569, 570, 571, 573, 574, 575, 576, 578, 579, 580, 581, 583, 584, 585, 586, 588, 589, 590, 591, 593, 594, 595, 596, 598, 599, 600, 601, 603, 604, 605, 606, 608, 609, 610, or 611. The PBS can be, for example, 5 to 19 nucleotides in length and can comprise the sequence corresponding to any one of sequence numbers 518, 519, 520, 521, 522, 523, 524, 525, 526, 527, 528, 529, 530, 531, or 532. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 26 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 26 and a gRNA core capable of complexing with a Cas9 protein. For example, the sequence in the spacer of the ngRNA can comprise nucleotides 4-20, 3-20, 2-20, or 1-20 of sequence number 376, 144, 613, 614, 615, 616, 617, 379, 380, or 381. In some embodiments, the spacer of the ngRNA is a ngRNA spacer listed in Table 26. The ngRNA spacers in Table 26 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 27 provides Prime Editing guide RNAs (PEgRNAs) that can be used with any Prime Editor containing a Cas9 protein capable of recognizing a CAG PAM sequence. The PEgRNAs of Table 27 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 27 comprise: (a) a spacer comprising at its 3′ end a sequence corresponding to sequence number 734; (b) a gRNA core capable of complexing with a Cas9 protein, and (c) an extension arm comprising: (i) an editing template at least 39 nucleotides in length and comprising at its 3′ end a sequence corresponding to any one of sequence numbers 739 or 740, and (ii) a prime binding site (PBS) comprising at its 5′ end a sequence corresponding to sequence number 719. The PEgRNA spacer can be, for example, 17-22 nucleotides in length and can comprise the sequence corresponding to any one of sequence numbers 734, 735, 736, 284, 737, or 738. In some embodiments, the PEgRNA spacer comprises sequence number 284. The PEgRNA spacers in Table 27 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 739 or 741. Alternatively, the editing template can encode one or more synonymous mutations relative to the wildtype USH2A gene. The editing template can encode one or more synonymous mutations that are PAM silencing mutations and can comprise at its 3′ end the sequence corresponding to sequence number 740 or 741. The PBS can be, for example, 5 to 19 nucleotides in length and can comprise the sequence corresponding to any one of sequence numbers 719, 720, 721, 722, 723, 724, 725, 726, 727, 728, 729, 730, 731, 732, or 733. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 27 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 27 and a gRNA core capable of complexing with a Cas9 protein. For example, the sequence in the spacer of the ngRNA can comprise nucleotides 4-20, 3-20, 2-20, or 1-20 of sequence number 743, 144, 744, 745, 746, 379, 668, 747, or 381. In some embodiments, the spacer of the ngRNA is a ngRNA spacer listed in Table 27. The ngRNA spacers in Table 27 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 28 provides Prime Editing guide RNAs (PEgRNAs) that can be used with any Prime Editor containing a Cas9 protein capable of recognizing a CAG PAM sequence. The PEgRNAs of Table 28 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 28 comprise: (a) a spacer comprising at its 3′ end a sequence corresponding to sequence number 763; (b) a gRNA core capable of complexing with a Cas9 protein, and (c) an extension arm comprising: (i) an editing template at least 20 nucleotides in length and comprising at its 3′ end a sequence corresponding to any one of sequence numbers 768, 769, 774, 775, 776, or 778, and (ii) a prime binding site (PBS) comprising at its 5′ end a sequence corresponding to sequence number 748. The PEgRNA spacer can be, for example, 17-22 nucleotides in length and can comprise the sequence corresponding to any one of sequence numbers 763, 764, 765, 285, 766, or 767. In some embodiments, the PEgRNA spacer comprises sequence number 285. The PEgRNA spacers in Table 28 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 769, 771, 773, 779, 785, 791, 797, 803, 809, 815, 821, 827, 833, 839, 845, 851, 857, 863, 869, 875, or 881. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 768, 770, 772, 774, 775, 776, 777, 778, 780, 781, 782, 783, 784, 786, 787, 788, 789, 790, 792, 793, 794, 795, 796, 798, 799, 800, 801, 802, 804, 805, 806, 807, 808, 810, 811, 812, 813, 814, 816, 817, 818, 819, 820, 822, 823, 824, 825, 826, 828, 829, 830, 831, 832, 834, 835, 836, 837, 838, 840, 841, 842, 843, 844, 846, 847, 848, 849, 850, 852, 853, 854, 855, 856, 858, 859, 860, 861, 862, 864, 865, 866, 867, 868, 870, 871, 872, 873, 874, 876, 877, 878, 879, or 880. The PBS can be, for example, 5 to 19 nucleotides in length and can comprise the sequence corresponding to any one of sequence numbers 748, 749, 750, 751, 752, 753, 754, 755, 756, 757, 758, 759, 760, 761, or 762. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 28 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 28 and a gRNA core capable of complexing with a Cas9 protein. For example, the sequence in the spacer of the ngRNA can comprise nucleotides 4-20, 3-20, 2-20, or 1-20 of sequence number 667, 144, 882, 883, 884, 746, 379, 668, 669, 747, 381. In some embodiments, the spacer of the ngRNA is a ngRNA spacer listed in Table 28. The ngRNA spacers in Table 28 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 28 annotated with the same * and number code as an RTT in Table 28 has perfect complementarity to the edit strand post-edit by a PEgRNA containing the RTT.

Table 29 provides Prime Editing guide RNAs (PEgRNAs) that can be used with any Prime Editor containing a Cas9 protein capable of recognizing a TG PAM sequence. The PEgRNAs of Table 29 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 29 comprise: (a) a spacer comprising at its 3′ end a sequence corresponding to sequence number 643; (b) a gRNA core capable of complexing with a Cas9 protein, and (c) an extension arm comprising: (i) an editing template at least 23 nucleotides in length and comprising at its 3′ end a sequence corresponding to sequence number 899, and (ii) a prime binding site (PBS) comprising at its 5′ end a sequence corresponding to sequence number 885. The PEgRNA spacer can be, for example, 17-22 nucleotides in length and can comprise the sequence corresponding to any one of sequence numbers 643, 644, 645, 646, 647, or 648. In some embodiments, the PEgRNA spacer comprises sequence number 646. The PEgRNA spacers in Table 29 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 899, 900, 901, 902, 903, 904, 905, 906, 907, 908, 909, 910, 911, 912, 913, 914, 915, or 916. The PBS can be, for example, 5 to 19 nucleotides in length and can comprise the sequence corresponding to any one of sequence numbers 885, 886, 887, 888, 889, 890, 670, 891, 892, 893, 894, 895, 896, 897, or 898. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 29 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 29 and a gRNA core capable of complexing with a Cas9 protein. For example, the sequence in the spacer of the ngRNA can comprise nucleotides 4-20, 3-20, 2-20, or 1-20 of sequence number 30, 31, 32, 33, 34, 35, 36, 139, 37, 38, 39, 41, 42, 43, 44, 45, 48, 49, 50, 51, 52, 53, or 54. In some embodiments, the spacer of the ngRNA is a ngRNA spacer listed in Table 29. The ngRNA spacers in Table 29 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit.

Table 30 provides Prime Editing guide RNAs (PEgRNAs) that can be used with any Prime Editor containing a Cas9 protein capable of recognizing a GAG, GAGG, or GAGGAT PAM sequence. The PEgRNAs of Table 30 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 30 comprise: (a) a spacer comprising at its 3′ end a sequence corresponding to sequence number 932; (b) a gRNA core capable of complexing with a Cas9 protein, and (c) an extension arm comprising: (i) an editing template at least 13 nucleotides in length and comprising at its 3′ end a sequence corresponding to any one of sequence numbers 937, 938, 943, or 945, and (ii) a prime binding site (PBS) comprising at its 5′ end a sequence corresponding to sequence number 917. The PEgRNA spacer can be, for example, 17-22 nucleotides in length and can comprise the sequence corresponding to any one of sequence numbers 932, 933, 934, 379, 935, or 936. In some embodiments, the PEgRNA spacer comprises sequence number 379. The PEgRNA spacers in Table 30 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 938, 940, 942, 946, 950, 954, 958, 962, 966, 970, 974, 978, 982, 986, 990, 994, 998, 1002, 1006, 1010, 1014, 1018, 1022, 1026, 1030, 1034, 1038, or 1042. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 937, 939, 941, 943, 944, 945, 947, 948, 949, 951, 952, 953, 955, 956, 957, 959, 960, 961, 963, 964, 965, 967, 968, 969, 971, 972, 973, 975, 976, 977, 979, 980, 981, 983, 984, 985, 987, 988, 989, 991, 992, 993, 995, 996, 997, 999, 1000, 1001, 1003, 1004, 1005, 1007, 1008, 1009, 1011, 1012, 1013, 1015, 1016, 1017, 1019, 1020, 1021, 1023, 1024, 1025, 1027, 1028, 1029, 1031, 1032, 1033, 1035, 1036, 1037, 1039, 1040, or 1041. The PBS can be, for example, 5 to 19 nucleotides in length and can comprise the sequence corresponding to any one of sequence numbers 917, 918, 919, 920, 921, 922, 923, 924, 925, 926, 927, 928, 929, 930, or 931. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 30 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 30 and a gRNA core capable of complexing with a Cas9 protein. For example, the sequence in the spacer of the ngRNA can comprise nucleotides 4-20, 3-20, 2-20, or 1-20 of sequence number 30, 282, 284, 36, 962, 285, 286, 1043, 287, 288, 289, 290, 291, 292, 293, 294, or 295. In some embodiments, the spacer of the ngRNA is a ngRNA spacer listed in Table 30. The ngRNA spacers in Table 30 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 30 annotated with the same * and number code as an RTT in Table 30 has perfect complementarity to the edit strand post-edit by a PEgRNA containing the RTT.

Table 31 provides Prime Editing guide RNAs (PEgRNAs) that can be used with any Prime Editor containing a Cas9 protein capable of recognizing an ATGG PAM sequence. The PEgRNAs of Table 31 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 31 comprise: (a) a spacer comprising at its 3′ end a sequence corresponding to sequence number 1059; (b) a gRNA core capable of complexing with a Cas9 protein, and (c) an extension arm comprising: (i) an editing template at least 26 nucleotides in length and comprising at its 3′ end a sequence corresponding to sequence number 1064, and (ii) a prime binding site (PBS) comprising at its 5′ end a sequence corresponding to sequence number 1044. The PEgRNA spacer can be, for example, 17-22 nucleotides in length and can comprise the sequence corresponding to any one of sequence numbers 1059, 1060, 1061, 1043, 1062, or 1063. In some embodiments, the PEgRNA spacer comprises sequence number 1043. The PEgRNA spacers in Table 31 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1064, 1065, 1066, 1067, 1068, 1069, 1070, 1071, 1072, 1073, 1074, 1075, 1076, 1077, or 1078. The PBS can be, for example, 5 to 19 nucleotides in length and can comprise the sequence corresponding to any one of sequence numbers 1044, 1045, 1046, 1047, 1048, 1049, 1050, 1051, 1052, 1053, 1054, 1055, 1056, 1057, or 1058. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 31 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 31 and a gRNA core capable of complexing with a Cas9 protein. For example, the sequence in the spacer of the ngRNA can comprise nucleotides 4-20, 3-20, 2-20, or 1-20 of sequence number 743, 667, 1079, 1080, or 379. In some embodiments, the spacer of the ngRNA is a ngRNA spacer listed in Table 31. The ngRNA spacers in Table 31 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 32 provides Prime Editing guide RNAs (PEgRNAs) that can be used with any Prime Editor containing a Cas9 protein capable of recognizing an AG PAM sequence. The PEgRNAs of Table 32 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 32 comprise: (a) a spacer comprising at its 3′ end a sequence corresponding to sequence number 1095; (b) a gRNA core capable of complexing with a Cas9 protein, and (c) an extension arm comprising: (i) an editing template at least 16 nucleotides in length and comprising at its 3′ end a sequence corresponding to any one of sequence numbers 1101 or 1105, and (ii) a prime binding site (PBS) comprising at its 5′ end a sequence corresponding to sequence number 1081. The PEgRNA spacer can be, for example, 17-22 nucleotides in length and can comprise the sequence corresponding to any one of sequence numbers 1095, 1096, 1097, 1098, 1099, or 1100. In some embodiments, the PEgRNA spacer comprises sequence number 1098. The PEgRNA spacers in Table 32 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1101, 1102, 1103, 1104, 36, 1107, 1109, 1111, 1113, 1115, 1117, 1119, 1121, 1123, 1125, 1127, 1129, 1131, 1133, 1135, 1137, 1139, 1141, 1143, or 1145. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 1105, 1106, 1108, 1110, 1112, 1114, 1116, 1118, 1120, 1122, 1124, 1126, 1128, 1130, 1132, 1134, 1136, 1138, 1140, 1142, 1144, or 1146. The PBS can be, for example, 5 to 19 nucleotides in length and can comprise the sequence corresponding to any one of sequence numbers 1081, 1082, 1083, 1084, 1085, 1086, 1087, 1088, 1089, 1090, 1091, 1092, 1093, 681, or 1094. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 32 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 32 and a gRNA core capable of complexing with a Cas9 protein. For example, the sequence in the spacer of the ngRNA can comprise nucleotides 4-20, 3-20, 2-20, or 1-20 of sequence number 30, 1147, 31, 33, 1148, 34, 35, 36, 1106, 139, 37, 38, 39, 41, 42, 1149, 43, 44, 140, 48, 49, 51, 52, 53, or 54. In some embodiments, the spacer of the ngRNA is a ngRNA spacer listed in Table 32. The ngRNA spacers in Table 32 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 32 annotated with the same * and number code as an RTT in Table 32 has perfect complementarity to the edit strand post-edit by a PEgRNA containing the RTT.

Table 33 provides Prime Editing guide RNAs (PEgRNAs) that can be used with any Prime Editor containing a Cas9 protein capable of recognizing a GAG PAM sequence. The PEgRNAs of Table 33 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 33 comprise: (a) a spacer comprising at its 3′ end a sequence corresponding to sequence number 1164; (b) a gRNA core capable of complexing with a Cas9 protein, and (c) an extension arm comprising: (i) an editing template at least 17 nucleotides in length and comprising at its 3′ end a sequence corresponding to any one of sequence numbers 1169, 1170, 1175, 1176, 1178, or 1179, and (ii) a prime binding site (PBS) comprising at its 5′ end a sequence corresponding to sequence number 1150. The PEgRNA spacer can be, for example, 17-22 nucleotides in length and can comprise the sequence corresponding to any one of sequence numbers 1164, 1165, 1166, 747, 1167, or 1168. In some embodiments, the PEgRNA spacer comprises sequence number 747. The PEgRNA spacers in Table 33 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1169, 1171, 1173, 1177, 1183, 1189, 1195, 1201, 1207, 1213, 1219, 1225, 1231, 1237, 1243, 1249, 1255, 1261, 1267, 1273, 1279, 1285, 1291, or 1297. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 1170, 1172, 1174, 1175, 1176, 1178, 1179, 1180, 1181, 1182, 1184, 1185, 1186, 1187, 1188, 1190, 1191, 1192, 1193, 1194, 1196, 1197, 1198, 1199, 1200, 1202, 1203, 1204, 1205, 1206, 1208, 1209, 1210, 1211, 1212, 1214, 1215, 1216, 1217, 1218, 1220, 1221, 1222, 1223, 1224, 1226, 1227, 1228, 1229, 1230, 1232, 1233, 1234, 1235, 1236, 1238, 1239, 1240, 1241, 1242, 1244, 1245, 1246, 1247, 1248, 1250, 1251, 1252, 1253, 1254, 1256, 1257, 1258, 1259, 1260, 1262, 1263, 1264, 1265, 1266, 1268, 1269, 1270, 1271, 1272, 1274, 1275, 1276, 1277, 1278, 1280, 1281, 1282, 1283, 1284, 1286, 1287, 1288, 1289, 1290, 1292, 1293, 1294, 1295, 1296, 1298, 1299, or 1300. The PBS can be, for example, 5 to 19 nucleotides in length and can comprise the sequence corresponding to any one of sequence numbers 1150, 1151, 1152, 1153, 1154, 1155, 1156, 1157, 1158, 1159, 1160, 1161, 679, 1162, or 1163. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 33 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 33 and a gRNA core capable of complexing with a Cas9 protein. For example, the sequence in the spacer of the ngRNA can comprise nucleotides 4-20, 3-20, 2-20, or 1-20 of sequence number 284, 285, 287, 1301, 1302, 1303, 1304, 1305, 1306, or 295. In some embodiments, the spacer of the ngRNA is a ngRNA spacer listed in Table 33. The ngRNA spacers in Table 33 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 33 annotated with the same * and number code as an RTT in Table 33 has perfect complementarity to the edit strand post-edit by a PEgRNA containing the RTT.

Table 34 provides Prime Editing guide RNAs (PEgRNAs) that can be used with any Prime Editor containing a Cas9 protein capable of recognizing an AG or AGA PAM sequence. The PEgRNAs of Table 34 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 34 comprise: (a) a spacer comprising at its 3′ end a sequence corresponding to sequence number 1321; (b) a gRNA core capable of complexing with a Cas9 protein, and (c) an extension arm comprising: (i) an editing template at least 18 nucleotides in length and comprising at its 3′ end a sequence corresponding to any one of sequence numbers 1327, 1329, or 1330, and (ii) a prime binding site (PBS) comprising at its 5′ end a sequence corresponding to sequence number 1307. The PEgRNA spacer can be, for example, 17-22 nucleotides in length and can comprise the sequence corresponding to any one of sequence numbers 1321, 1322, 1323, 1324, 1325, or 1326. In some embodiments, the PEgRNA spacer comprises sequence number 1324. The PEgRNA spacers in Table 34 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1327, 1328, 1303, 1331, 1334, 1337, 1340, 1343, 1346, 1349, 1352, 1355, 1358, 1361, 1364, 1367, 1370, 1373, 1376, 1379, 1382, 1385, or 1388. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 1329, 1330, 1332, 1333, 1335, 1336, 1338, 1339, 1341, 1342, 1344, 1345, 1347, 1348, 1350, 1351, 1353, 1354, 1356, 1357, 1359, 1360, 1362, 1363, 1365, 1366, 1368, 1369, 1371, 1372, 1374, 1375, 1377, 1378, 1380, 1381, 1383, 1384, 1386, or 1387. The PBS can be, for example, 5 to 19 nucleotides in length and can comprise the sequence corresponding to any one of sequence numbers 1307, 1308, 1309, 1310, 1311, 1312, 1313, 1314, 1315, 1316, 1317, 677, 1318, 1319, or 1320. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 34 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 34 and a gRNA core capable of complexing with a Cas9 protein. For example, the sequence in the spacer of the ngRNA can comprise nucleotides 4-20, 3-20, 2-20, or 1-20 of sequence number 1389, 30, 31, 1390, 1391, 33, 34, 35, 1392, 1393, 36, 139, 37, 38, 39, 41, 1394, 1395, 42, 43, 44, 1396, 1397, 45, 48, 49, 51, 52, 53, or 54. In some embodiments, the spacer of the ngRNA is a ngRNA spacer listed in Table 34. The ngRNA spacers in Table 34 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 34 annotated with the same * and number code as an RTT in Table 34 has perfect complementarity to the edit strand post-edit by a PEgRNA containing the RTT.

Table 35 provides Prime Editing guide RNAs (PEgRNAs) that can be used with any Prime Editor containing a Cas9 protein capable of recognizing a CAG or CAGAGT PAM sequence. The PEgRNAs of Table 35 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 35 comprise: (a) a spacer comprising at its 3′ end a sequence corresponding to sequence number 1412; (b) a gRNA core capable of complexing with a Cas9 protein, and (c) an extension arm comprising: (i) an editing template at least 19 nucleotides in length and comprising at its 3′ end a sequence corresponding to any one of sequence numbers 1417, 1418, 1422, 1423, 1424, or 1425, and (ii) a prime binding site (PBS) comprising at its 5′ end a sequence corresponding to sequence number 1398. The PEgRNA spacer can be, for example, 17-22 nucleotides in length and can comprise the sequence corresponding to any one of sequence numbers 1412, 1413, 1414, 381, 1415, or 1416. In some embodiments, the PEgRNA spacer comprises sequence number 381. The PEgRNA spacers in Table 35 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1418, 42, 1420, 1426, 1432, 1438, 1444, 1450, 1456, 1462, 1468, 1474, 1480, 1486, 1492, 1498, 1504, 1510, 1516, 1522, 1528, or 1534. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 1417, 1394, 1419, 1421, 1422, 1423, 1424, 1425, 1427, 1428, 1429, 1430, 1431, 1433, 1434, 1435, 1436, 1437, 1439, 1440, 1441, 1442, 1443, 1445, 1446, 1447, 1448, 1449, 1451, 1452, 1453, 1454, 1455, 1457, 1458, 1459, 1460, 1461, 1463, 1464, 1465, 1466, 1467, 1469, 1470, 1471, 1472, 1473, 1475, 1476, 1477, 1478, 1479, 1481, 1482, 1483, 1484, 1485, 1487, 1488, 1489, 1490, 1491, 1493, 1494, 1495, 1496, 1497, 1499, 1500, 1501, 1502, 1503, 1505, 1506, 1507, 1508, 1509, 1511, 1512, 1513, 1514, 1515, 1517, 1518, 1519, 1520, 1521, 1523, 1524, 1525, 1526, 1527, 1529, 1530, 1531, 1532, or1533. The PBS can be, for example, 5 to 19 nucleotides in length and can comprise the sequence corresponding to any one of sequence numbers 1398, 1399, 1400, 1401, 1402, 1403, 1404, 1405, 1406, 1407, 675, 1408, 1409, 1410, or 1411. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 35 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 35 and a gRNA core capable of complexing with a Cas9 protein. For example, the sequence in the spacer of the ngRNA can comprise nucleotides 4-20, 3-20, 2-20, or 1-20 of sequence number 282, 284, 1392, 1393, 36, 1535, 1536, 1537, 285, 286, 287, 288, 290, 1301, 1302, 1538, 1539, 1540, 1303, 291, 292, 293, 294, or 295. In some embodiments, the spacer of the ngRNA is a ngRNA spacer listed in Table 35. The ngRNA spacers in Table 35 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 35 annotated with the same * and number code as an RTT in Table 35 has perfect complementarity to the edit strand post-edit by a PEgRNA containing the RTT.

Table 36 provides Prime Editing guide RNAs (PEgRNAs) that can be used with any Prime Editor containing a Cas9 protein capable of recognizing a GG or GGA PAM sequence. The PEgRNAs of Table 36 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 36 comprise: (a) a spacer comprising at its 3′ end a sequence corresponding to sequence number 1895; (b) a gRNA core capable of complexing with a Cas9 protein, and (c) an extension arm comprising: (i) an editing template at least 11 nucleotides in length and comprising at its 3′ end a sequence corresponding to any one of sequence numbers 1901 or 1904, and (ii) a prime binding site (PBS) comprising at its 5′ end a sequence corresponding to sequence number 1880. The PEgRNA spacer can be, for example, 17-22 nucleotides in length and can comprise the sequence corresponding to any one of sequence numbers 1895, 1896, 1897, 1898, 1899, or 1900. In some embodiments, the PEgRNA spacer comprises sequence number 1898. The PEgRNA spacers in Table 36 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 1901, 1902, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, or 1957. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, or 1956. The PBS can be, for example, 5 to 19 nucleotides in length and can comprise the sequence corresponding to any one of sequence numbers 1880, 1881, 1882, 1883, 1884, 1885, 1886, 1887, 1888, 1889, 1890, 1891, 1892, 1893, or 1894.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 36 can comprise a sequence corresponding to any one of sequence numbers 1958 to 1989. Any PEgRNA exemplified in Table 36 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 36 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 36 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 36 and a gRNA core capable of complexing with a Cas9 protein. For example, the sequence in the spacer of the ngRNA can comprise nucleotides 4-20, 3-20, 2-20, or 1-20 of sequence number 30, 31, 34, 138, 35, 139, 37, 38, 39, 41, 43, 44, 140, 48, 49, 51, 52, 53, or 54. In some embodiments, the spacer of the ngRNA is a ngRNA spacer listed in Table 36. The ngRNA spacers in Table 36 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 36 annotated with the same * and number code as an RTT in Table 36 has perfect complementarity to the edit strand post-edit by a PEgRNA containing the RTT.

Table 37 provides Prime Editing guide RNAs (PEgRNAs) that can be used with any Prime Editor containing a Cas9 protein capable of recognizing an AG or AGG PAM sequence. The PEgRNAs of Table 37 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 37 comprise: (a) a spacer comprising at its 3′ end a sequence corresponding to sequence number 2005; (b) a gRNA core capable of complexing with a Cas9 protein, and (c) an extension arm comprising: (i) an editing template at least 12 nucleotides in length and comprising at its 3′ end a sequence corresponding to any one of sequence numbers 2011, and (ii) a prime binding site (PBS) comprising at its 5′ end a sequence corresponding to sequence number 1990. The PEgRNA spacer can be, for example, 17-22 nucleotides in length and can comprise the sequence corresponding to any one of sequence numbers 2005, 2006, 2007, 2008, 2009, or 2010. In some embodiments, the PEgRNA spacer comprises sequence number 2008. The PEgRNA spacers in Table 37 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, or 2039. The PBS can be, for example, 5 to 19 nucleotides in length and can comprise the sequence corresponding to any one of sequence numbers 1990, 1991, 1992, 1993, 1994, 1995, 1996, 1997, 1998, 1999, 2000, 2001, 2002, 2003, or 2004.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 37 can comprise a sequence corresponding to any one of sequence numbers 2040 to 2071 or 7535 to 7542. Any PEgRNA exemplified in Table 37 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 37 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 37 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 37 and a gRNA core capable of complexing with a Cas9 protein. For example, the sequence in the spacer of the ngRNA can comprise nucleotides 4-20, 3-20, 2-20, or 1-20 of sequence number 2039, 30, 31, 33, 34, 138, 35, 139, 37, 38, 39, 41, 43, 44, 140, 48, 49, 51, 52, 53, or 54. In some embodiments, the spacer of the ngRNA is a ngRNA spacer listed in Table 37. The ngRNA spacers in Table 37 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit.

Table 38 provides Prime Editing guide RNAs (PEgRNAs) that can be used with any Prime Editor containing a Cas9 protein capable of recognizing a TG or TGA PAM sequence. The PEgRNAs of Table 38 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 38 comprise: (a) a spacer comprising at its 3′ end a sequence corresponding to sequence number 2087; (b) a gRNA core capable of complexing with a Cas9 protein, and (c) an extension arm comprising: (i) an editing template at least 14 nucleotides in length and comprising at its 3′ end a sequence corresponding to any one of sequence numbers 2093, 2096, 2097, or 2098, and (ii) a prime binding site (PBS) comprising at its 5′ end a sequence corresponding to sequence number 2072. The PEgRNA spacer can be, for example, 17-22 nucleotides in length and can comprise the sequence corresponding to any one of sequence numbers 2087, 2088, 2089, 2090, 2091, or 2092. In some embodiments, the PEgRNA spacer comprises sequence number 2090. The PEgRNA spacers in Table 38 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 2093, 2094, 2095, 2099, 2103, 2107, 30, 2114, 2118, 2122, 2126, 2130, 2134, 2138, 2142, 2146, 2150, 2154, 2158, 2162, 2166, 2170, 2174, 2178, 2182, 2186, or 2190. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 2096, 2097, 2098, 2100, 2101, 2102, 2104, 2105, 2106, 2108, 2109, 2110, 2111, 2112, 2113, 2115, 2116, 2117, 2119, 2120, 2121, 2123, 2124, 2125, 2127, 2128, 2129, 2131, 2132, 2133, 2135, 2136, 2137, 2139, 2140, 2141, 2143, 2144, 2145, 2147, 2148, 2149, 2151, 2152, 2153, 2155, 2156, 2157, 2159, 2160, 2161, 2163, 2164, 2165, 2167, 2168, 2169, 2171, 2172, 2173, 2175, 2176, 2177, 2179, 2180, 2181, 2183, 2184, 2185, 2187, 2188, or 2189. The PBS can be, for example, 5 to 19 nucleotides in length and can comprise the sequence corresponding to any one of sequence numbers 2072, 2073, 2074, 2075, 2076, 2077, 2078, 2079, 2080, 2081, 2082, 2083, 2084, 2085, or 2086.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 38 can comprise a sequence corresponding to any one of sequence numbers 2191 to 3086 or 7523 to 7534. Any PEgRNA exemplified in Table 38 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 38 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 38 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 38 and a gRNA core capable of complexing with a Cas9 protein. For example, the sequence in the spacer of the ngRNA can comprise nucleotides 4-20, 3-20, 2-20, or 1-20 of sequence number 30, 31, 33, 34, 138, 35, 36, 139, 37, 38, 39, 41, 43, 44, 140, 48, 49, 51, 52, 53, or 54. In some embodiments, the spacer of the ngRNA is a ngRNA spacer listed in Table 38. The ngRNA spacers in Table 38 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 38 annotated with the same * and number code as an RTT in Table 38 has perfect complementarity to the edit strand post-edit by a PEgRNA containing the RTT.

Table 39 provides Prime Editing guide RNAs (PEgRNAs) that can be used with any Prime Editor containing a Cas9 protein capable of recognizing an AG PAM sequence. The PEgRNAs of Table 39 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 39 comprise: (a) a spacer comprising at its 3′ end a sequence corresponding to sequence number 4496; (b) a gRNA core capable of complexing with a Cas9 protein, and (c) an extension arm comprising: (i) an editing template at least 38 nucleotides in length and comprising at its 3′ end a sequence corresponding to sequence number 4501, and (ii) a prime binding site (PBS) comprising at its 5′ end a sequence corresponding to sequence number 4481. The PEgRNA spacer can be, for example, 17-22 nucleotides in length and can comprise the sequence corresponding to any one of sequence numbers 4496, 4497, 4498, 51, 4499, or 4500. In some embodiments, the PEgRNA spacer comprises sequence number 51. The PEgRNA spacers in Table 39 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 4501, 4502, or 4503. The PBS can be, for example, 5 to 19 nucleotides in length and can comprise the sequence corresponding to any one of sequence numbers 4481, 4482, 4483, 4484, 4485, 4486, 4487, 4488, 4489, 4490, 4491, 4492, 4493, 4494, or 4495. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 39 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 39 and a gRNA core capable of complexing with a Cas9 protein. For example, the sequence in the spacer of the ngRNA can comprise nucleotides 4-20, 3-20, 2-20, or 1-20 of sequence number 4, 58, 1898, 4508, 4509, 4510, 4511, 4512, 2008, 4513, 4514, 4515, 4516, 1676, 4517, 4518, 4519, 646, 617, 4520, 4521, 4522, 3564, 1736, 4523, 4524, 2090, 380, 1098, 4525, or 1324. In some embodiments, the spacer of the ngRNA is a ngRNA spacer listed in Table 39. The ngRNA spacers in Table 39 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 39 can comprise a sequence corresponding to sequence number 4504, 4505, 4506, or 4507.

Table 40 provides Prime Editing guide RNAs (PEgRNAs) that can be used with any Prime Editor containing a Cas9 protein capable of recognizing a TG or TGA PAM sequence. The PEgRNAs of Table 40 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 40 comprise: (a) a spacer comprising at its 3′ end a sequence corresponding to sequence number 4541; (b) a gRNA core capable of complexing with a Cas9 protein, and (c) an extension arm comprising: (i) an editing template at least 17 nucleotides in length and comprising at its 3′ end a sequence corresponding to any one of sequence numbers 4546, 4549, 4550, or 4551, and (ii) a prime binding site (PBS) comprising at its 5′ end a sequence corresponding to sequence number 4526. The PEgRNA spacer can be, for example, 17-22 nucleotides in length and can comprise the sequence corresponding to any one of sequence numbers 4541, 4542, 4543, 48, 4544, or 4545. In some embodiments, the PEgRNA spacer comprises sequence number 48. The PEgRNA spacers in Table 40 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 4546, 4547, 4548, 4511, 4553, 4557, 4561, 4565, 4569, 4573, 4577, 4581, 4585, 4589, 4593, 4597, 4601, 4605, 4609, 4613, 4617, 4621, 4625, or 4629. Alternatively, the editing template can encode one or more synonymous mutations relative to the wildtype USH2a gene. The editing template can encode one or more synonymous mutations that are PAM silencing mutations and can comprise at its 3′ end the sequence corresponding to sequence number 4549, 4550, 4551, 4552, 4554, 4555, 4556, 4558, 4559, 4560, 4562, 4563, 4564, 4566, 4567, 4568, 4570, 4571, 4572, 4574, 4575, 4576, 4578, 4579, 4580, 4582, 4583, 4584, 4586, 4587, 4588, 4590, 4591, 4592, 4594, 4595, 4596, 4598, 4599, 4600, 4602, 4603, 4604, 4606, 4607, 4608, 4610, 4611, 4612, 4614, 4615, 4616, 4618, 4619, 4620, 4622, 4623, 4624, 4626, 4627, 4628, 4630, or 4631. The PBS can be, for example, 5 to 19 nucleotides in length and can comprise the sequence corresponding to any one of sequence numbers 4526, 4527, 4528, 4529, 4530, 4531, 4532, 4533, 4534, 4535, 4536, 4537, 4538, 4539, or 4540.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 40 can comprise a sequence corresponding to any one of sequence numbers 4621 to 5806 or 7519 to 7522. Any PEgRNA exemplified in Table 40 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 40 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 40 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 40 and a gRNA core capable of complexing with a Cas9 protein. For example, the sequence in the spacer of the ngRNA can comprise nucleotides 4-20, 3-20, 2-20, or 1-20 of sequence number 4, 58, 1898, 4509, 4510, 4549, 4511, 4550, 4551, 2008, 4634, 4635, 1676, 4519, 646, 4636, 4522, 4637, 4638, 4639, 3564, 1736, 4640, 2090, 380, 1098, or 1324. In some embodiments, the spacer of the ngRNA is a ngRNA spacer listed in Table 40. The ngRNA spacers in Table 40 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 40 annotated with the same * and number code as an RTT in Table 40 has perfect complementarity to the edit strand post-edit by a PEgRNA containing the RTT. Exemplary ngRNA provided in Table 40 can comprise a sequence corresponding to sequence number 4504, 4632, 4505, 4506, 4633, or 4507.

Table 41 provides Prime Editing guide RNAs (PEgRNAs) that can be used with any Prime Editor containing a Cas9 protein capable of recognizing an AG PAM sequence. The PEgRNAs of Table 41 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 41 comprise: (a) a spacer comprising at its 3′ end a sequence corresponding to sequence number 5822; (b) a gRNA core capable of complexing with a Cas9 protein, and (c) an extension arm comprising: (i) an editing template at least 19 nucleotides in length and comprising at its 3′ end a sequence corresponding to sequence number 5827, and (ii) a prime binding site (PBS) comprising at its 5′ end a sequence corresponding to sequence number 5807. The PEgRNA spacer can be, for example, 17-22 nucleotides in length and can comprise the sequence corresponding to any one of sequence numbers 5822, 5823, 5824, 37, 5825, or 5826. In some embodiments, the PEgRNA spacer comprises sequence number 37. The PEgRNA spacers in Table 41 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 5827, 5828, 5829, 5830, 5831, 5832, 5833, 5834, 5835, 5836, 5837, 5838, 5839, 5840, 5841, 5842, 5843, 5844, 5845, 5846, 5847, or 5848. The PBS can be, for example, 5 to 19 nucleotides in length and can comprise the sequence corresponding to any one of sequence numbers 5807, 5808, 5809, 5810, 5811, 5812, 5813, 5814, 5815, 5816, 5817, 5818, 5819, 5820, or 5821. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 41 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 41 and a gRNA core capable of complexing with a Cas9 protein. For example, the sequence in the spacer of the ngRNA can comprise nucleotides 4-20, 3-20, 2-20, or 1-20 of sequence number 4, 58, 1898, 4508, 4509, 4510, 4511, 2008, 4634, 4635, 1676, 4519, 646, 4522, 4639, 3564, 1736, 4640, 2090, 380, 1098, or 1324. In some embodiments, the spacer of the ngRNA is a ngRNA spacer listed in Table 41. The ngRNA spacers in Table 41 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 41 can comprise a sequence corresponding to sequence number 4504, 4632, 4505, 4506, 4633, or 4507.

Table 42 provides Prime Editing guide RNAs (PEgRNAs) that can be used with any Prime Editor containing a Cas9 protein capable of recognizing a TG or TGG PAM sequence. The PEgRNAs of Table 42 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 42 comprise: (a) a spacer comprising at its 3′ end a sequence corresponding to sequence number 5864; (b) a gRNA core capable of complexing with a Cas9 protein, and (c) an extension arm comprising: (i) an editing template at least 25 nucleotides in length and comprising at its 3′ end a sequence corresponding to sequence number 5869, and (ii) a prime binding site (PBS) comprising at its 5′ end a sequence corresponding to sequence number 5849. The PEgRNA spacer can be, for example, 17-22 nucleotides in length and can comprise the sequence corresponding to any one of sequence numbers 5864, 5865, 5866, 38, 5867, 5868. In some embodiments, the PEgRNA spacer comprises sequence number 38. The PEgRNA spacers in Table 42 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 5869, 5870, 5871, 5872, 5873, 5874, 5875, 5876, 5877, 5878, 5879, 5880, 5881, 5882, 5883, or 5884. The PBS can be, for example, 5 to 19 nucleotides in length and can comprise the sequence corresponding to any one of sequence numbers 5849, 5850, 5851, 5852, 5853, 5854, 5855, 5856, 5857, 5858, 5859, 5860, 5861, 5862, or 5863.

The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule. Exemplary PEgRNAs provided in Table 42 can comprise a sequence corresponding to any one of sequence numbers 5885 to 5916 or 7515 to 7518. Any PEgRNA exemplified in Table 42 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 42 may alternatively be adapted for expression from a DNA template, for example, by including a 5′ terminal G if the spacer of the PEgRNA begins with another nucleotide, by including 6 or 7 U nucleotides at the 3′ end of the extension arm, or both. Such expression-adapted sequences may further comprise a hairpin-forming motif between the PBS and the 3′ terminal U series.

Any of the PEgRNAs of Table 42 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 42 and a gRNA core capable of complexing with a Cas9 protein. For example, the sequence in the spacer of the ngRNA can comprise nucleotides 4-20, 3-20, 2-20, or 1-20 of sequence number 4, 58, 1898, 4508, 4509, 4510, 4511, 4512, 2008, 4634, 4635, 1676, 4519, 646, 617, 4522, 3564, 1736, 4640, 2090, 380, 1098, 4525, or 1324. In some embodiments, the spacer of the ngRNA is a ngRNA spacer listed in Table 42. The ngRNA spacers in Table 42 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 42 can comprise a sequence corresponding to sequence number 4504, 4632, 4505, 4506, 4633, or 4507.

Table 43 provides Prime Editing guide RNAs (PEgRNAs) that can be used with any Prime Editor containing a Cas9 protein capable of recognizing a GG PAM sequence. The PEgRNAs of Table 43 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 43 comprise: (a) a spacer comprising at its 3′ end a sequence corresponding to sequence number 5991; (b) a gRNA core capable of complexing with a Cas9 protein, and (c) an extension arm comprising: (i) an editing template at least 24 nucleotides in length and comprising at its 3′ end a sequence corresponding to sequence number 5996, and (ii) a prime binding site (PBS) comprising at its 5′ end a sequence corresponding to sequence number 5976. The PEgRNA spacer can be, for example, 17-22 nucleotides in length and can comprise the sequence corresponding to any one of sequence numbers 5991, 5992, 5993, 39, 5994, or 5995. In some embodiments, the PEgRNA spacer comprises sequence number 39. The PEgRNA spacers in Table 43 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 5996, 5997, 5998, 5999, 6000, 6001, 6002, 6003, 6004, 6005, 6006, 6007, 6008, 6009, 6010, 6011, or 6012. The PBS can be, for example, 5 to 19 nucleotides in length and can comprise the sequence corresponding to any one of sequence numbers 5976, 5977, 5978, 5979, 5980, 5981, 5982, 5983, 5984, 5985, 5986, 5987, 5988, 5989, or 5990. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 43 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 43 and a gRNA core capable of complexing with a Cas9 protein. For example, the sequence in the spacer of the ngRNA can comprise nucleotides 4-20, 3-20, 2-20, or 1-20 of sequence number 4, 58, 1898, 4508, 4509, 4510, 4511, 4512, 2008, 4634, 4635, 1676, 4519, 646, 617, 4522, 4639, 3564, 1736, 4640, 2090, 380, 1098, 4525, or 1324. In some embodiments, the spacer of the ngRNA is a ngRNA spacer listed in Table 43. The ngRNA spacers in Table 43 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 43 can comprise a sequence corresponding to sequence number 1324, 4504, 4632, 4505, 4506, 4633, or 4507.

Table 44 provides Prime Editing guide RNAs (PEgRNAs) that can be used with any Prime Editor containing a Cas9 protein capable of recognizing a TG PAM sequence. The PEgRNAs of Table 44 can also be used in Prime Editing systems further comprising a nick guide RNA (ngRNA). Such PEgRNAs and Prime Editing systems can be used, for example, to correct a c.2276 G->T mutation in USH2a.

The PEgRNAs exemplified in Table 44 comprise: (a) a spacer comprising at its 3′ end a sequence corresponding to sequence number 7084; (b) a gRNA core capable of complexing with a Cas9 protein, and (c) an extension arm comprising: (i) an editing template at least 36 nucleotides in length and comprising at its 3′ end a sequence corresponding to any one of sequence numbers 7089, and (ii) a prime binding site (PBS) comprising at its 5′ end a sequence corresponding to sequence number 7069. The PEgRNA spacer can be, for example, 17-22 nucleotides in length and can comprise the sequence corresponding to any one of sequence numbers 7084, 7085, 7086, 52, 7087, or 7088. In some embodiments, the PEgRNA spacer comprises sequence number 52. The PEgRNA spacers in Table 44 are annotated with their PAM sequence(s), enabling the selection of an appropriate Cas9 protein. The editing template can be referred to as a reverse transcription template (RTT). The editing template can encode wildtype USH2a gene sequence. For example, the editing template can comprise at its 3′ end the sequence corresponding to sequence number 7089, 7090, 7091, 7092, or 7093. The PBS can be, for example, 5 to 19 nucleotides in length and can comprise the sequence corresponding to any one of sequence numbers 7069, 7070, 7071, 7072, 7073, 7074, 7075, 7076, 7077, 7078, 7079, 7080, 7081, 7082, or 7083. The PEgRNA can comprise, from 5′ to 3′, the spacer, the gRNA core, the edit template, and the PBS. The 3′ end of the edit template can be contiguous with the 5′ end of the PBS. The PEgRNA can comprise multiple RNA molecules or can be a single RNA molecule.

Any of the PEgRNAs of Table 44 can be used in a Prime Editing system further comprising a nick guide RNA (ngRNA). Such ngRNA can comprise a spacer comprising at its 3′ end a sequence corresponding to nucleotides 4-20 of any ngRNA spacer listed in Table 44 and a gRNA core capable of complexing with a Cas9 protein. For example, the sequence in the spacer of the ngRNA can comprise nucleotides 4-20, 3-20, 2-20, or 1-20 of sequence number 4, 58, 1898, 4508, 4509, 4510, 4511, 4512, 2008, 4634, 4515, 4635, 1676, 4517, 4519, 646, 617, 4520, 4522, 3564, 1736, 4523, 2090, 380, 1098, 4525, or 1324. In some embodiments, the spacer of the ngRNA is a ngRNA spacer listed in Table 44. The ngRNA spacers in Table 44 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the USH2a gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. Exemplary ngRNA provided in Table 44 can comprise a sequence corresponding to sequence number 4504, 4632, 4505, 4506, 4633, or 4507.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, tale magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., an USH2A gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., an USH2A gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g., the target USH2A gene, upon the contacting of the PE composition with the target gene. In some embodiments, the DNA binding domain of the PE associates with the PEgRNA. In some embodiments, the PE binds the target gene, e.g., an USH2A gene, directed by the PEgRNA. Accordingly, in some embodiments, the contacting of the target gene result in binding of a DNA binding domain of a prime editor of the target USH2A gene directed by the PEgRNA.

In some embodiments, contacting the target gene with the prime editing composition results in a nick in an edit strand of the target gene, e.g., an USH2A gene by the prime editor upon contacting with the target gene, thereby generating a nicked on the edit strand of the target gene. In some embodiments. contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene, e.g., an USH2A gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN]. In some embodiments, the method further comprises contacting the target gene with a flap endonuclease. In some embodiments, the flap endonuclease is provided as a pail of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g., an USH12A gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRiNA after contacting the target gene with the PE. In some embodiments, the target gene is contacted with the ngRNA and/or the PEgRNA before contacting the target gene with the prime editor.

In some embodiments, the target gene, e.g., an USH2A gene, is in a cell. Accordingly, also provided herein are methods of modifying a cell.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs). viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In soie embodiments, the method comprises introducing the PEgRNA or the polynucleotide encoding the PEgRNA, the polynucleotide encoding the prime editor polypeptide, and/or the ngRNA or the polynucleotide encoding the ngRN into the cell sequentially. In some embodiments, the method comprises introducing the polynucleotide encoding the prime editor poly peptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRN A may be introduced into the cell by any delivery approaches described herein or any delivery approach known in the art, for example, by RNPs, LNPs, viral vectors, non-viral vectors. mRNA delivery, and physical. In some embodiments, the polynicleotide is a DNA polynucleotide. In some embodiments, the polynucleotide is a RNA polynucleotide, e.g., mRNA polynucleotide.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA. and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a stem cell. In some embodiments, the cell is a human stem cell. In some embodiments, the cell is an induced pluripotent stem cell (iPSC). In some embodiments, the cell is a human iPSC. In some embodiments, the cell is a neuron. In some embodiments, the cell is a neuron from basal ganglia. In some embodiments, the cell is a neuron from basal ganglia of a subject. In some embodiments, the cell is at least one of: Miller cells, astrocytes, or microglia. In some embodiments, the cell edited by prime editing can be differentiated into, or give rise to recovery of a population of cells, e.g., sensory ciliated cells, retinal cells, photoreceptor cells, rod cells, cone cells, hair cells, post-natal hair cells, human sensory ciliated cells, human retinal cells, human photoreceptor cells, human rod cells, human cone cells, human hair cells, or human post-natal hair cells.

In some embodiments, the cell is an ex vivo cell. In some embodiments, the cell is an ex vivo cell obtained from a human subject. For example, in some embodiments, the cell is a stem cell, a progenitor cell obtained from a subject having Usher syndrome type 2 disease prior to editing. After correction of the mutation by prime editing, the cell may be administered to the subject. In some embodiments, the cell is in a subject, e.g., a human subject.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In sonic embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In sonic embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRiNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours. 36 hours, 48 hours, 3 days, 4 days. 5 days, 7 days, 10 days, or 14 days of exposing a target gene (e.g., a USH2A gene within the genome of a cell) to a prime editing composition. In some embodiments, editing efficiency of the prime editing compositions and method described herein can be measured by calculating the percentage of edited target genes in a population of cells introduced with the prime editing composition. In some embodiments, the editing efficiency is determined after 5, 6, 7, 8, 9, 10, 11, 12, 13, 14, 15. or 16 weeks of exposing a target gene (e.g., a USH2A gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control. In some embodiments. editing efficiency of prime the prime editing compositions and method described herein can be measured by calculating the percentage of edited target genes in a population of cells after in vivo engraftment of the edited cells. In some embodiments, the editing efficiency is determined after 5, 6, 7, 8, 9, 10, 11, 12, 13, 14, 15, or 16 weeks of engraftment. In some embodiments, the editing efficiency is determined after 8 or 16 weeks of engraftment. In some embodiments, prime editing is able to maintain in edited cells at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more than 95% of editing efficiency after 8 or 16 weeks post engraftment.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell (as measured in a population of primary cells) relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporatₑd herein in its entirety. In some embodiments, the prime editing methods disclosed herein can have an indel frequency of less than 30%, less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a USH2A gene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than 1% in a target cell, e.g., a human retinal cell, a human rod cell, a human cone cell, a human hair cell, or a human iPSCa human retinal cell, a human rod cell, a human cone cell, a human hair cell, or a human iPSC. In some embodiments, the prime editing methods disclosed herein have an editing efficiency of at least about 1% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 1% and an indel frequency of less than 0.1% in a target cell, e.g., a human retinal cell, a human rod cell, a human cone cell, a human hair cell, or a human iPSC. In some embodiments, the prime editing methods disclosed herein have an editing efficiency of at least about 5% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 5% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 5% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 7.5% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 7.5% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 7.5% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 10% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 10% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 10% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 15% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 15% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 15% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 20% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 20% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 20% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 30% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 30% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 30% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 40% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 40% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 40% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 50% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 50% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 50% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 60% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 60% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 60% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 70% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 70% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 70% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 80% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 80% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 80% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 90% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 90% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 90% and an indel frequency of less than 0.1% in a target cell, e.g., a human retinal cell, a human rod cell, a human cone cell, a human hair cell, or a human iPSC.

In some embodiments, the prime editing methods disclosed herein have an editing efficiency of at least about 95% and an indel frequency of less than 1% in a target cell, e.g., a human retinal cell, a human rod cell, a human cone cell, a human hair cell, or a human iPSC. In some embodiments, the prime editing methods disclosed herein have an editing efficiency of at least about 95% and an indel frequency of less than 0.5% in a target cell, e.g., a human retinal cell, a human rod cell, a human cone cell, a human hair cell, or a human iPSC. In some embodiments, the prime editing methods disclosed herein have an editing efficiency of at least about 95% and an indel frequency of less than 0.1% in a target cell, e.g., a human retinal cell, a human rod cell, a human cone cell, a human hair cell, or a human iPSC. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a USH2A gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a USH2A gene within the genome of a cell) to a prime editing composition. In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition. In some embodiments, the prime editing compositions (e.g., PEgRNAs and prime editors as described herein) and prime editing methods disclosed herein can be used to edit a target USH2A gene. In some embodiments, the target USH2A gene comprises a mutation compared to a wild type USH24 gene. In some embodiments, the mutation is associated with Usher syndrome type 2. In some embodiments, the target USH2A gene comprises an editing target sequence that contains the mutation associated with Usher syndrome type 2. In some embodiments, the mutation is associated with nsRP. In some embodiments, the target US2A gene comprises an editing target sequence that contains the mutation associated with nsRP. In some embodiments, the mutation is in a coding region of the target USH2A gene. In some embodiments, the mutation is in an exon of the target USH2A gene. In some embodiments, the prime editing method comprises contacting a target USH2A gene with a prime editing composition comprising a prime editor, a PEgRNA, and/or a ngRNA. In some embodiments, contacting the target USH2A gene with the prime editing composition results in incorporation of one or more intended nucleotide edits in the target USH42A gene. In some embodiments, the incorporation is in a region of the target USH2A gene that corresponds to an editing target sequence in the USH2A gene. In some embodiments, the one or more intended nucleotide edits comprises a single nucleotide substitution, an insertion, a deletion, or any combination thereof, compared to the endogenous sequence of the target USH2A gene. In some embodiments, incorporation of the one or more intended nucleotide edits results in replacement of one or more mutations with the corresponding sequence that encodes a wild type usherin polypeptide set forth in SEQ ID NO: 7598. In some embodiments, incorporation of the one or more intended nucleotide edits results in replacement of the one or more mutations with the corresponding sequence in a wild type USH2A gene. In some embodiments, incorporation of the one more intended nucleotide edits results in correction of a mutation in the target USH2A gene. In some embodiments, the target USH2A gene comprises an editing template sequence that contains the mutation. In some embodiments, contacting the target USH2A gene with the prime editing composition results in incorporation of one or more intended nucleotide edits in the target USH2A gene, which corrects the mutation in the editing target sequence (or a double stranded region comprising the editing target sequence and the complementary sequence to the editing target sequence on a target strand) in the target USH2A gene.

In some embodiments, incorporation of the one more intended nucleotide edits results in correction of a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, or exon 72 of the USH2A gene as compared to a wild type USH2A gene. In some embodiments, incorporation of the one more intended nucleotide edits results in correction of a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 of the USH2A gene as compared to a wild type USH2A gene. In some embodiments, incorporation of the one more intended nucleotide edits results in correction of a mutation at a splice site (i.e. an intron/exon junction) of the USH2A gene as compared to a wild type USH2A gene. In some embodiments, incorporation of the one more intended nucleotide edits results in correction of a mutation in an intron of the USH2A gene as compared to a wild type USH2A gene.

In some embodiments, incorporation of the one or more intended nucleotide edits results in correction of an USH2A gene sequence and restores wild type expression and function of the wild type usherin protein. In some embodiments, incorporation of the one or more intended nucleotide edits results in correction of an USH2A gene sequence and restores wild type expression and function of the wild type usherin isoform b protein. In some embodiments, incorporation of the one or more intended nucleotide edits results in correction of an USH2A gene sequence and restores wild type expression and function of the wild type usherin isoform a protein. In some embodiments, incorporation of the one or more intended nucleotide edits does not correct the mutation in the editing target sequence to wild type sequence, but allows for expression of a functional usherin protein, e.g., a functional isoform b or a functional isoform a, of the usherin protein, encoded by the USH2A gene. For example, in some embodiments, incorporation of the one or more intended nucleotide edits results in one or more codons that are different from a wild type codon but encode one or more amino acids same as the wild type usherin protein. In some embodiments, incorporation of the one or more intended nucleotide edits results in one or more codons that encode one or more amino acids different from the wild type usherin protein, but allows for expression of a functional usherin protein.

In some embodiments, incorporation of the one or more intended nucleotide edits results in correction of a mutation that is in exon 13 of the USH2A gene as compared to a wild type USH2A1 gene. In some embodiments, incorporation of the one or more intended nucleotide edits results in correction of a mutation in the USH2A gene that is located between positions 216246995 and 216247195 of human chromosome 1. In some embodiments, incorporation of the one or more intended nucleotide edits results in correction of a E767 frameshift (E767fs) mutation in the usherin protein encoded by the USH2A gene as compared to a wild type usherin protein as set forth in SEQ ID N0:7598 and restores expression and function of a wild type usherin protein. In some embodiments, incorporation of the one or more intended nucleotide edits results in correction of a deletion of nucleotide G at a position corresponding to position 216247095 of human chromosome 1 (c.2299delG mutation) in the USH2A gene as compared to a wild type USH2A gene. In some embodiments, incorporation of the one or more intended nucleotide edits results in correction of a deletion of nucleotide C at a position corresponding to position 216247095 of human chromosome 1 as compared to a wild type USH2A gene. In some embodiments, the one or more nucleotide edits incorporated in the USH2A gene is an insertion of nucleotide G (or an insertion of nucleotide C in a complementary strand) at a position corresponding to position 216247095 of human chromosome 1 as compared to the endogenous sequence of the USH2A gene.

In some embodiments, incorporation of the one or more intended nucleotide edits results in correction of a mutation in the USH2A gene that is located between positions 216247018 and 216247218 of human genome 1. In some embodiments, incorporation of the one or more intended nucleotide edits results in correction of a mutation that results in a C759F amino acid substitution in the usherin protein encoded by the USH2A gene as compared to a wild type usherin protein as set forth in SEQ ID NO:7598 and restores expression and function of a wild type usherin protein. In some embodiments, incorporation of the one or more intended nucleotide edits results in correction of a G→T nucleotide substitution in the USH2A gene at a position corresponding to position 216247118 of human chromosome 1 (c.2276G→T mutation) as compared to a wild type USH2A gene. In some embodiments, incorporation of the one or more intended nucleotide edits results in correction of a C→A nucleotide substitution in the USH2A gene at a position corresponding to position 216247118 of human chromosome 1 as compared to a wild type USH2A gene. In some embodiments, the one or more nucleotide edits incorporated in the USH2A gene is a T→C nucleotide substitution (or a A→C nucleotide substitution in a complementary strand) at a position corresponding to position 216247118 of human chromosome 1 as compared to the endogenous sequence of the USH2A gene.

In some embodiments, incorporation of the one or more intended nucleotide edits results in correction of a mutation that is in intron 40 of the USH24 gene as compared to a wild type USH2A gene. In some embodiments, incorporation of the one or more intended nucleotide edits results in correction of a mutation that is located between positions 215891098 and 216247218 of human genome 1. In some embodiments, incorporation of the one or more intended nucleotide edits results in correction of a mutation that results in inclusion of a pseudo exon between exon 40 and exon 41 into a USH2A mRNA encoded by the USH2A gene as compared to a wild type USH2A mRNA, and restores expression of a wild type USH2A mRNA, and expression and function of a wild type usherin protein. In some embodiments, incorporation of the one or more intended nucleotide edits results in correction of a A→G nucleotide substitution in the USH2A gene at a position corresponding to position 215891198 of human chromosome 1 (c.7595-2144 A→G mutation) as compared to a wild type US1I2A gene. In some embodiments incorporation of the one or more intended nucleotide edits results in correction of a T→C nucleotide substitution in the USH2A gene at a position corresponding to position 215891198 of human chromosome 1 as compared to a wild type USH2A gene. In some embodiments, the one or more nucleotide edits incorporated in the USH2A gene is a G→A nucleotide substitution (or a C→T nucleotide substitution in a complementary strand) at a position corresponding to position 215891198 of human chromosome 1 as compared to the endogenous sequence of the USH2A gene.

In some embodiments, incorporation of the one or more intended nucleotide edits results in correction of a mutation that is in exon 61 of the USH2A gene as compared to a wild type USH2A gene. In some embodiments, incorporation of the one or more intended nucleotide edits results in correction of a mutation that is located between positions 215728132 and 215728332 as set forth in human genome 1. In some embodiments, incorporation of the one or more intended nucleotide edits results in correction of a mutation that results in an amino acid substitution W3955* in the usherin protein encoded by the USH2A gene as compared to a wild type usherin protein as set forth in SEQ ID NO:7598, wherein * refers to a premature stop codon, and wherein incorporation of the one or more intended nucleotide edits restores expression and function of a wild type usherin protein. In some embodiments, incorporation of the one or more intended nucleotide edits results in correction of a G→A nucleotide substitution in the USH2A gene at a position corresponding to position 216247118 of human chromosome 1 (c.11864G→A mutation) as compared to a wild type US112A gene. In some embodiments, incorporation of the one or more intended nucleotide edits results in correction of a C→T nucleotide substitution in the USH2A gene at a position corresponding to position 216247118 of human chromosome 1 as compared to a wild type USH2A gene. In some embodiments, the one or more nucleotide edits incorporated in the USH2A gene is a A→G nucleotide substitution in the USH2A gene (or a T→C nucleotide substitution in a complementary strand) at a position corresponding to position 216247118 of human chromosome 1 as compared to the endogenous sequence of the USH2A gene.

In some embodiments, the target USH2A gene is in a target cell. Accordingly, in one aspect provided herein is a method of editing a target cell comprising a target USH2A gene that encodes a polypeptide that comprises one or more mutations relative to a wild type USH2A gene. In some embodiments, the methods of the present disclosure comprise introducing a prime editing composition comprising a PEgRNA, a prime editor polypeptide, and/or a ngRNA into the target cell that has the target USH2A gene to edit the target USH2A gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the cell is an induced pluripotent stem cell (iPSC). In some embodiments, the cell is a human induced pluripotent stem cell (hiPSC). In some embodiments, the cell is an embryonic stem cell (ESC). In some embodiments, the cell is a human embryonic stem cell. In some embodiments, the target cell is a retinal cell. In some embodiments, the target cell is a human retinal cell. In some embodiments, the target cell is a photoreceptor cell. In some embodiments, the target cell is a human photoreceptor cell. In some embodiments, the target cell is a rod cell. In some embodiments, the target cell is a human rod cell. In some embodiments, the target cell is a cone cell. In some embodiments, the target cell is a human cone cell. In some embodiments, the target cell is a retinal pigment epithelium cell. In some embodiments, the target cell is a human retinal pigment epithelium cell. In some embodiments, the target cell is a hair cell. In some embodiments, the target cell is a human hair cell. In some embodiments, the target cell is a human retinal cell derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. In some embodiments, the cell edited by prime editing can be differentiated into, or give rise to recovery of a population of cells. In some embodiments, the target cell is an ex vivo cell. In some embodiments, the target cell is an ex vivo cell obtained from a human subject. In some embodiments, the target cell is in a subject, e.g., a human subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target USH2A gene that comprises one or more mutations restores wild type expression and function of the usherin protein encoded by the USH2A gene. In some embodiments, the target USH2A gene comprises a frameshift mutation (c.2299delG mutation) in exon 13, which results in premature stop codon as compared to a wild type usherin protein prior to incorporation of the one or more intended nucleotide edits. In some embodiments, the target US1I2A gene encodes a C759F (resulted from c.2276G→T mutation) amino acid substitution as compared to a wild type usherin protein prior to incorporation of the one or more intended nucleotide edits. In some embodiments, incorporation of the one or more nucleotide edits restores expression and/or function of a wild type usherin protein at least partially or completely. In some embodiments, incorporation of the one or more nucleotide edits restores expression and/or function of a wild type usherin protein isoform a at least partially or completely. In some embodiments, incorporation of the one or more nucleotide edits restores expression and/or function of a wild type usherin protein isoform b at least partially or completely. In some embodiments, expression and/or function of the usherin protein may be measured when expressed in a target cell. In some embodiments, incorporation of the one or more intended nucleotide edits in the target USH2A gene comprising one or more mutations lead to a fold change in a level of USH2A gene expression, usherin protein expression, or a combination thereof. In some embodiments, a change in the level of usherin isoform a expression level can comprise a fold change of, e.g., 2-fold. 3-fold. 4-fold, 5-fold, 6-fold, 7-fold, 8-fold, 9-fold, 10-fold, 15-fold, 20-fold, 25-fold, 30-fold. 40-fold, 50-fold, 60-fold, 70-fold, 80-fold, 90-fold, 100-fold or greater as compared to expression in a suitable control cell not introduced with a prime editing composition described herein. In some embodiments, a change in the level of usherin isoform b expression level can comprise a fold change of, e.g., 2-fold, 3-fod, 4-fold, 5-fold, 6-fold, 7-fold, 8-fold, 9-fold, 10-fold, 15-fold, 20-fold, 25-fold, 30-fold, 40-fold, 50-fold. 60-fold, 70-fold. 80-fold, 90-fold, 100-fold or greater as compared to expression in a suitable control cell not introduced with a prime editing composition described herein. In some embodiments, incorporation of the one or more intended nucleotide edits in the target USH2A gene that comprises one or more mutations restores wild type expression of the usherin protein by at least 10%, 20%, 30%, 40%, 50%, 60%, 70%, 80%, 90%, 95%, or 99% or more as compared to wild type expression of the usherin protein in a suitable control cell that comprises a wild type US112A gene.

In some embodiments, incorporation of the one or more intended nucleotide edits in the target USH2A gene that comprises one or more mutations or nucleotide alterations results in expression of a usherin polypeptide encoded by the target USH2A gene. In some embodiments, incorporation of the one or more intended nucleotide edits in the target USH2A gene that comprises one or more mutations or nucleotide alterations results in an edited USH2A gene that has a wild type USH2A gene sequence. In some embodiments, incorporation of the one or more intended nucleotide edits in the target USH2A that comprises one or more mutations or nucleotide alterations results in expression of a functional usherin polypeptide. In some embodiments, incorporation of the one or more intended nucleotide edits in the target USH2A gene that comprises one or more mutations or nucleotide alterations results in expression of a functional usherin polypeptide that has the same sequence as a wild type usherin polypeptide. In some embodiments, expression and/or function of the usherin polypeptide may be measured when expressed in a target cell. In some embodiments, incorporation of the one or more intended nucleotide edits in the target USH2A gene leads to a fold change in a level of USH2A transcript expression, usherin protein expression, or a combination thereof. In some embodiments, a change in the level of USH2A transcript expression level can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target USH2 A gene that comprises one or more mutations restores wild type expression of the usherin protein isoform a by at least 10%, 20%, 30%, 40%, 50%, 60%, 70%, 80%, 90%, 95%, o99% or more as compared to wild type expression of the usherin protein isoform a in a suitable control cell that comprises a wild type USH2A gene. In some embodiments, incorporation of the one or more intended nucleotide edits in the target USH2A gene that comprises one or more mutations restores wild type expression of the usherin protein isoform b by at least 10%, 20%, 30%, 40%, 50%, 60%, 70%, 80%, 90%, 95%, o99% or more as compared to wild type expression of the usherin protein isoform b in a suitable control cell that comprises a wild type USH2A gene.

In some embodiments, an usherin protein or USH2A gene expression increase can be measured by a functional assay. In some embodiments, protein expression can be measured using a protein assay. In some embodiments, protein expression can be measured using antibody testing. In some embodiments, an antibody can comprise anti-usherin antibody. In some embodiments, protein expression can be measured using ELISA, mass spectrometry, Western blot, sodium dodecyl sulfate poly acrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel. Expression and function of usherin protein can be examined ex vivo or in vivo. In some embodiments, usherin protein expression in target cells or target organs, e.g., photoreceptors, or particular regions such as periciliary region of photoreceptors, can be examined by, e.g., immunofluorescent assay using an antibody that specifically recognizes usherin, in some embodiments, expression and activity can be measured by examining expression and co-localization of proteins involved in the function of usherin protein net work, for example, whirlin. In some embodiments, activity of the unsherin protein can be examined in vivo by measuring restoration of retinal function, for example, by visual motor response (VNMR) assay.

Methods of Treating Usher Syndrome and nsRP

In some embodiments, provided herein are methods for treatment of a subject diagnosed with a disease associated with or caused by one or more pathogenic mutations. In some embodiments, provided herein are methods for treatment of a subject diagnosed with a disease associated with or caused by one or more pathogenic mutations that can be corrected by prime editing. In some embodiments, methods of treatment provided herein comprises editing one or more genes other than the gene that harbors the one or more pathogenic mutations. In some embodiments, provided herein are methods for treating Usher syndrome type 2 that comprise administering to a subject a therapeutically effective amount of a prime editing composition, or a pharmaceutical composition comprising a prime editing composition as described herein. In some embodiments, provided herein are methods for treating non-syndromic retinitis pigmentosa (nsRP) that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the target gene in the subject. In some embodiments, administration of the prime editing composition results in correction of one or more pathogenic mutations, e.g., point mutations, insertions, or deletions, associated with Usher syndrome type 2 in the subject. In some embodiments, administration of the prime editing composition results in correction of one or more pathogenic mutations, e.g., point mutations, insertions, or deletions, associated with nsRP in the subject. In some embodiments, the target gene comprise an editing target sequence that contains the pathogenic mutation. In some embodiments, administration of the prime editing composition results in incorporation of one or more intended nucleotide edits in the target gene that corrects the pathogenic mutation in the editing target sequence (or a double stranded region comprising the editing target sequence and the complementary sequence to the editing target sequence on a target strand) of the target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the USH2A gene in a subject, e.g., a human subject, suffering from, having, susceptible to, or at risk for Usher syndrome type 2 or nsRP. Identifying a subject in need of such treatment can be in the judgment of a subject or a health care professional and can be subjective (e.g., opinion) or objective (e.g., measurable by a test or diagnostic method). In some embodiments, the subject has Usher syndrome type 2. In some embodiments, the subject has nsRP.

In some embodiments, the subject has been diagnosed with Usher syndrome type 2 or nsRP by sequencing of a USH2A gene in the subject. In some embodiments, the subject comprises at least a copy of USH2A gene that comprises one or more mutations compared to a wild type USH2A gene. In some embodiments, the subject comprises at least a copy of USH2A gene that comprises a mutation in a coding region of the USH2A gene as compared to a wild type USH2A gene. In some embodiments, the subject comprises at least a copy of USH2A gene that comprises a mutation in a non-coding region as compared to a wild type USH2A gene. In some embodiments, the subject comprises two copies of USH2A gene each comprising a mutation in a coding region of the USH12A gene as compared to a wild type US-12A gene. In some embodiments, the subject comprises two copies of USH2A gene each comprising a mutation in a non-coding region of the USH2A gene as compared to a wild type USH2A gene.

In some embodiments, the subject comprises at least a copy of USH2A gene that comprises a mutation as compared to a wild type USH2A gene wherein the mutation is associated with Usher syndrome type 2. In some embodiments, the subject comprises two copies of USH2A gene each comprising a mutation as compared to a wild type USH2A gene wherein the mutation is associated with Usher syndrome type 2. In some embodiments, the mutation is a c.2299delG mutation that results in a E767 frameshift mutation and a premature stop codon in the usherin protein encoded by the USH2A gene. In some embodiments, the subject comprises at least a copy of USH2A gene that comprises a mutation as compared to a wild type USH2A gene wherein the mutation is associated with nsRP. In some embodiments, the subject comprises two copies of USH2A gene each comprising a mutation as compared to a wild type USH2A gene wherein the mutation is associated with nsRP. In some embodiments, the mutation is a c.2276 mutation that results in a E767 frameshift mutation and a premature stop codon in the usherin protein encoded by the USH2A gene. In some embodiments, administrating to the subject an effective amount of a prime editing composition described herein corrects the mutation in the at least one copy of USH2A gene in the subject. In some embodiments, administrating to the subject an effective amount of a prime editing composition described herein corrects the mutation in both copies of US-12A gene in the subject. In some embodiments, administrating to the subject an effective amount of a prime editing composition described herein restores expression and function of a functional usherin protein in the subject. In some embodiments, administrating to the subject an effective amount of a prime editing composition described herein restores expression and/or function of a wild type usherin protein in the subject.

In some embodiments, the subject comprises at least a copy of USH2A gene that comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, or exon 72 as compared to a wild type USH2A gene. In some embodiments, the subject comprises at least a copy of USH2A gene that comprises a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 as compared to a wild type US112A gene. In some embodiments, the subject comprises two copies of USH12A gene each comprising a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, or exon 72 as compared to a wild type USH2A gene. In some embodiments, the subject comprises two copies of USH2A gene each comprising a mutation in exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, or exon 21 as compared to a wild type UST-42A gene. In some embodiments, the subject comprises at least a copy of USH2A gene that comprises a mutation at a splice site (i.e. an intron/exon junction) as compared to a wild type USH2A gene. In some embodiments, the subject comprises two copies of USH2A gene each comprising a mutation a mutation at a splice site (i.e. an intron/exon junction) as compared to a wild type USH2A gene. In some embodiments, the subject comprises at least a copy of USH2A gene that comprises a mutation in an intron as compared to a wild type USH2A gene. In some embodiments, the subject comprises two copies of USH2A gene each comprising a mutation in an intron as compared to a wild type USH2A gene. In some embodiments, the subject comprises two copies of USH2A gene, wherein each of the two copies comprises a same mutation, e.g., nucleotide deletion, insertion, substitution, or any mutation described herein or known in the art, wherein the mutation is associated with Usher syndrome type 2 or nsRP. In some embodiments, the subject comprises two copies of USH2A gene, wherein the two copies each comprises a different mutation, e.g., nucleotide deletion, insertion, substitution, or any mutation described herein or known in the art, wherein the mutation is associated with Usher syndrome type 2 or nsRP. In some embodiments, administrating to the subject an effective amount of a prime editing composition described herein corrects the mutation in at least one of the two copies of USH2A gene in the subject. In some embodiments, administrating to the subject an effective amount of a prime editing composition described herein corrects the mutation(s) in both copies of USH2A gene in the subject. In some embodiments, administrating to the subject an effective amount of a prime editing composition described herein restores expression and function of a functional usherin protein in the subject. In some embodiments, administrating to the subject an effective amount of a prime editing composition described herein restores expression and/or function of a wild type usherin protein in the subject. In some embodiments, the prime editing composition comprises a single PEgRNA. In some embodiments, the prime editing composition comprises two or more PEgR NAs each capable of correcting a different mutation in the USH2A gene.

In some embodiments, the subject comprises at least a copy of USH2A gene that comprises a mutation in exon 13 as compared to a wild type USH2A gene. In some embodiments, the subject comprises at least a copy of USH2A gene that comprises a mutation located between positions 216246995 and 216247195 of human chromosome 1 as compared to a wild type USH2A gene. In some embodiments, the subject comprises at least a copy of US112A gene that comprises a mutation that results in a E767 frameshift (E767fs) mutation in the usherin protein encoded by the USH2A gene as compared to a wild type usherin protein as set forth in SEQ ID NO:7598. In some embodiments, the subject comprises at least a copy of USH2A gene that comprises a deletion of nucleotide G at a position corresponding to position 216247095 of human chromosome 1 (c.2299de1G mutation) as compared to a wild type US112A gene. In some embodiments, the subject comprises two copies of USH2A gene each comprising a mutation in exon 13 as compared to a wild type USH2A gene. In some embodiments, the subject comprises two copies of USH12A gene each comprising a mutation located between positions 216246995 and 216247195 of human chromosome 1 as compared to a wild type USH2A gene. In some embodiments, the subject comprises two copies of USH2A gene each comprising a mutation that results in a E767 frameshift (E767fs) mutation in the usherin protein encoded by the USH2A gene as compared to a wild type usherin protein as set forth in SEQ ID N0:7598. In some embodiments, the subject comprises two copies of USH2A gene each comprising a deletion of nucleotide G at a position corresponding to position 216247095 of human chromosome 1 (c.2299de1G mutation) as compared to a wild type USH2A gene. In some embodiments, administrating to the subject an effective amount of a prime editing composition described herein corrects the mutation in at least one of the two copies of USH2A gene in the subject. In some embodiments, administrating to the subject an effective amount of a prime editing composition described herein corrects the mutation(s) in both copies of USH2A gene in the subject. In some embodiments, administrating to the subject an effective amount of a prime editing composition described herein restores expression and/or function of a wild type usherin protein in the subject.

In some embodiments, the subject comprises at least a copy of USH2A gene that comprises a mutation located between positions 216247018 and 216247218 of human chromosome 1 as compared to a wild type USH2A gene. In some embodiments, the subject comprises at least a copy of USH2A gene that comprises a mutation that results in a C759F amino acid substitution in the usherin protein encoded by the USH2A gene as compared to a wild type usherin protein as set forth in SEQ ID NO:7598. In some embodiments, the subject comprises at least a copy of USH2A gene that comprises a G→T nucleotide substitution at a position corresponding to position 216247118 of human chromosome 1 (c.2276G→T mutation) as compared to a wild type USH2A gene. In some embodiments, the subject comprises two copies of USH2A gene each comprising a mutation located between positions 216247018 and 216247218 of human chromosome 1 as compared to a wild type USH2A gene. In some embodiments, the subject comprises two copies of USH2A gene each comprising a mutation that results in a C759F amino acid substitution in the usherin protein encoded by the USH2A gene as compared to a wild type usherin protein as set forth in SEQ ID NO:7598. In some embodiments, the subject comprises two copies of USH2A gene each comprising a G→T nucleotide substitution at a position corresponding to position 216247118 of human chromosome 1 (c.2276G→T mutation) as compared to a wild type USH12A gene. In some embodiments, administrating to the subject an effective amount of a prime editing composition described herein corrects the mutation in at least one of the two copies of USH2A gene in the subject. In some embodiments, administrating to the subject an effective amount of a prime editing composition described herein corrects the mutation(s) in both copies of USH2A gene in the subject. In some embodiments, administrating to the subject an effective amount of a prime editing composition described herein restores expression and/or function of a wild type usherin protein in the subject.

In some embodiments, the subject comprises at least a copy of USH2A gene that comprises a mutation in intron 40 as compared to a wild type USH2A gene. In some embodiments, the subject comprises at least a copy of USH2A gene that comprises a mutation located between positions 215891098 and 215891298 of human chromosome 1 as compared to a wild type USH2A gene. In some embodiments, the subject comprises at least a copy of US112A gene that comprises a mutation that results in inclusion of a pseudo exon between exon 40 and exon 41 into a USH2A mRNA encoded by the USH2A gene as compared to a wild type USH2A mRNA. In some embodiments, the subject comprises at least a copy of USH2A gene that comprises a A→G nucleotide substitution at a position corresponding to position 215891198 of human chromosome 1 (c.7595-2144 A→G mutation) as compared to a wild type USH2-gene. In some embodiments, the subject comprises two copies of USH2A gene each comprising a mutation in intron 40 as compared to a wild type USH2A gene. In some embodiments, the subject comprises two copies of USH2A gene each comprising a mutation located between positions 215891098 and 215891298 of human chromosome 1 as compared to a wild type USH2A gene. In some embodiments, the subject comprises two copies of USH2A gene each comprising a mutation that results in inclusion of a pseudo exon between exon 40 and exon 41 into a USH2A mRNA encoded by the USH2A gene as compared to a wild type USH2A mRNA. In some embodiments, the subject comprises two copies of USH2A gene each comprising a A→G nucleotide substitution at a position corresponding to position 215891198 of human chromosome 1 (c.7595-2144 A→G mutation) as compared to a wild type USH2A gene. In some embodiments, administrating to the subject an effective amount of a prime editing composition described herein corrects the mutation in at least one of the two copies of USH2A gene in the subject. In some embodiments, administrating to the subject an effective amount of a prime editing composition described herein corrects the mutation(s) in both copies of USH2A gene in the subject. III some embodiments, administrating to the subject an effective amount of a prime editing composition described herein restores expression and/or function of a wild type usherin protein in the subject.

In some embodiments, the subject comprises at least a copy of USH2A gene that comprises a mutation in exon 61 of the USH2A gene as compared to a wild type USH2A gene. In some embodiments, the subject comprises at least a copy of USH2A gene that comprises a mutation located between positions 215728132 and 215728332 of human chromosome 1 as compared to a wild type USH2A gene. In some embodiments, the subject comprises at least a copy of US-12A gene that comprises a mutation that results in an amino acid substitution W3955* in the usherin protein encoded by the USH2A gene as compared to a wild type usherin protein as set forth in SEQ ID NO:7598, wherein * refers to a premature stop codon. In some embodiments, the subject comprises at least a copy of USH2A gene that comprises a G→A nucleotide substitution at a position corresponding to position 215728232 of human chromosome 1 (c.11864G→A mutation) as compared to a wild type USH2A gene.

In some embodiments, the subject comprises two copies of USH2A gene each comprising a mutation in exon 61 of the USH2A gene as compared to a wild type USH2A gene. In some embodiments, the subject comprises two copies of USH2A gene each comprising a mutation located between positions 215728132 and 215728332 of human chromosome 1 as compared to a wild type USH2A gene. In some embodiments, the subject comprises two copies of USH2A gene each comprising a mutation that results in an amino acid substitution W3955* in the usherin protein encoded by the USH2A gene as compared to a wild type usherin protein as set forth in SEQ ID NO:7598, wherein * refers to a premature stop codon. In some embodiments, the subject comprises two copies of USH2A gene each comprising a G→A nucleotide substitution at a position corresponding to position 215728232 of human chromosome 1 (c.11864G→A mutation) as compared to a wild type USH2A gene.

In some embodiments, administrating to the subject an effective amount of a prime editing composition described herein corrects the mutation in at least one of the two copies of USH2A gene in the subject. In some embodiments, administrating to the subject an effective amount of a prime editing composition described herein corrects the mutation(s) in both copies of USH2A gene in the subject. In some embodiments, administrating to the subject an effective amount of a prime editing composition described herein restores expression and/or function of a wild type usherin protein in the subject.

In some embodiments, a population of patients each having one or more mutations in the target gene (e.g., an USH2A gene) may be treated with a prime editing composition (e.g., a PEgRNA, a prime editor, and optionally an ngRNA as described herein) disclosed herein. In some embodiments, a population of patients with different mutations in the target gene can be treated with the same prime editing composition comprising a single PEgRNA, a prime editor, and optionally an ngRNA. In some embodiments, a single prime editing composition comprising a single PEgRNA and a prime editor can be used to correct one or more, or two or more, mutations in the target gene in a populations of patients, wherein one or more patients in the population have different mutations from one another. In some embodiments, the prime editing composition comprising a single pair of PEgRNA, a prime editor, and optionally an ngRNA can be used to correct 2, 3, 4, 5, 6, 7, 8, 9, 10, 15, 20, 25 or more mutations in the target gene in a population of patients, wherein one or more patients in the population have different mutations from one another. In some embodiments, the PEgRNA comprises an editing template comprising a wild type sequence of the gene.

In some embodiments, a patient with multiple mutations in the target USH2A gene can be treated with a prime editing composition (e.g., a PEgRNAs, a prime editor, and optionally an ngRNA as described herein). For example, in some embodiments, a subject may comprise two copies of the gene, each comprising one or more different mutations. In some embodiments, a patient with one or more different mutations in the target gene can be treated with a single prime editing composition comprising a PEgRNAs, a prime editor, and optionally an ngRNA.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection or infusion into a subject. In some embodiments, the compositions described herein are administered by direct injection. In some embodiments, the compositions described herein are administered by subretinal injection. In some embodiments, the compositions described herein are administered by injection to the fovea or parafoveal regions. In some embodiments, the compositions described herein are administered by injection to peripheral regions of the retina. In some embodiments, the compositions described herein are administered by injection through the round window. In some embodiments, the compositions described herein are administered to the retina. In some embodiments, the compositions described herein are administered to the inner ear. In some embodiments, the compositions described herein are administered by injection to the cochlear duct. In some embodiments, the compositions described herein are administered by injection to the perilymph-filled spaces surrounding the cochlear duct, scala tympani and scala vestibuli, through the oval or round window membrane. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are progenitor cell. In some embodiments, the edited cells are induced pluripotent stem cells. In some embodiments, the edited cells are hepatocytes. In some embodiments, the edited cells are primary human cells. In some embodiments, the edited cells are human progenitor cells. In some embodiments, the edited cells are human stem cells. In some embodiments, the edited cells are human hepatocytes. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. In some embodiments, the edited cells are an ex vivo cells. In some embodiments, the edited cells are an ex vivo cells obtained from a human subject. In some embodiments, the edited cells are in a subject, e.g., a human subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in USH2A gene, or diagnostic measurement associated with Usher syndrome type 2 or nsRP in a subject suffering from the disease symptoms and has been administered an effective amount of a prime editing composition described herein. The level of the diagnostic marker determined in the method can be compared to known levels of the marker in either healthy normal controls or in other afflicted subjects to establish the subject's disease status.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In sonic embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA. e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “CC”, and guide poly nucleotide sequence. In some embodiments, the prime editor encoding nRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target USH2A gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, electroporation, nucleofection, microinjection, biolistics, virosomes, liposomes, immunoliposomes, polycation or lipid:nucleic acid conjugates, nanoparticles, cell penetrating peptides and associated conjugated molecules and chemistry, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered after delivery (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and .psi.2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiment, the polynucleotides are a DNA polynucleotide. In some embodiment, the poly nucleotides are an RNA polynucleotide; e.g., an mRNA polynucleotide.

In some embodiments, the AAV vector is selected for tropism to a particular cell, tissue, organism. In some embodiments, the AAV vector is pseudotyped, e.g. AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. In some embodiments, intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

In some embodiments, the in vivo use of dual AAV vectors results in the expression of full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c, a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Cas protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gRNA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 7607). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-I rev protein, nona-arginine (SEQ ID NO: 7623), and octa-arginine (SEQ ID NO: 7624). The nona-arginine (R9) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In sonic embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 55 below.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRN) form a ribonucleoprotein (R-3) for deliver to a target cell. In some embodiments, the RIP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 55 below.

TABLE 55
Exemplary lipids for nanoparticle formulation or gene transfer

| Lipid | Abbreviation | Featuure |
|---|---|---|
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N41-(2,3-Dioleyloxy)prophyliN,N,N- | DOTMA | Cationic |
| trimethylammonium chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3-bis | GAP-DLRIE | Cationic |
| (dodecyloxy)-1-propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethyl- | 2Oc | Cationic |
| pyridinium | | |
| 2,3-Dioleyloxy-N-P(spenninecarboxamido- | DOSPA | Cationic |
| ethy1J-N,Ndimethyl-1-propanatinium | | |
| trilfuoroacetate | | |
| 1,2-Dioley1-3-trimethylamtonium-propane | DOPA | Cationic |
| N-(2-Hydroxyethyl)-N,N-dimethy1-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N’, N’-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O’-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl | CCS | Cationic |
| carbamoyl-spenmine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl]imidazolinium chloride | | |
| N1-Cholesteryloxycarbonyl-3,7-diazanonane- | CDAN | Cationic |
| 1,9-diamine | | |
| 2-(3-Bis(3-amino-propy1)-amino]propylamino- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]- | DLin- | Cationic |
| dioxolane | KC2-DMA | |
| dilinoleyl-methyl-4-dimethylaminobutyrate | DLin- | Cationic |
| | MC3-DMA | |

Exemplary polymers for use in nanoparticle formulations and/or gene transfer are shown in Table 56 below

TABLE 56
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 57 below.

TABLE 57
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., electroporation, | YES | Transient | NO | Nucleic Acids |
| | particle gun, Calcium | | | | and Proteins |
| | phosphate transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very Transient | NO | DNA |
| | Herpes Simplex Virus | YES | Stable | NO | DNA |
| Non-Viral | Cationic | YES | Transient | Depends on what | Nucleic acids and |
| | | | | is delivered | Proteins |
| | Polymeric | YES | Transient | NO | Nucleic Acids |
| | Nanoparticles | | | | |
| Biological | Attenuated Bacteria | YES | Transient | NO | Nucleic Acids |
| Non-Viral | Engineered | YES | Transient | NO | Nucleic Acids |
| Delivery | Bacteriophages | | | | |
| Vehicles | Mammalian Virus-like | YES | Transient | NO | Nucleic Acids |
| | Particles | | | | |
| | Biological liposomes: | YES | Transient | NO | Nucleic Acids |
| | Erythrocyte Ghosts and | | | | |
| | Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or arty other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially. e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours. 2 hours. 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

The following examples are provided for illustrative purposes only and are not intended to limit the scope of the claims provided herein.

### Example 1—General Methods and Cell Line Production

PEgRNA assembly: PEgRNAs were chemically synthesized and modifications were introduced according to manufacturer's standard procedure (Integrated DNA Technologies). Alternatively, PEgRNA may be cloned with any of the following methods: in the first method, pooled synthesized DNA oligos encoding the PEgRNA and flanking U6 expression plasmid homology regions may be cloned into U6 expression plasmids via Gibson cloning and sequencing of bacterial colonies via Sanger or Next-generation sequencing. In the second method, double-stranded linear DNA fragments encoding PEgRNA and homology sequences as above may be individually Gibson-cloned into U6 expression plasmids. In the third method, for each PEgRNA, separate oligos encoding a protospacer, a gRNA scaffold, and PEgRNA extension (PBS and RTT) may be ligated, and then cloned into a U6 expression plasmid as described in Anzalone et al., Nature. 2019 December; 576(7785):149-157. Bacterial colonies carrying sequence-verified plasmids are propagated in LB or TB. Plasmid DNA can be purified by minipreps for mammalian transfection.

Where indicated, chemically synthesized PEgRNAs are modified at the 5′ end and the 3′end: the three 5′ most nucleotides were modified to phosphorothioated 2′-O-methyl nucleotides. The three consecutive nucleotides that precedes the 3′ most nucleotide (i.e. three consecutive nucleotides immediately 5′ of the last nucleotide at the 3′ end) were also modified to phosphorothioated 2′-O-methyl nucleotides.

HEK cell culture and transfection for PE2 and PE3 screen: HEK293T cells are propagated in DMEM with 10% FBS. Prior to transfection, cells are seeded in 96-well plates and then transfected with Lipofectamine 2000 or MessengerMax according to the manufacturer's directions with PEgRNA, mRNA encoding a Prime Editor, (and ngRNA for PE3 screen). Three days after transfection, gDNA is harvested in lysis buffer for high throughput sequencing and sequenced using Miseq.

USH2A 2299delG or 2276 G4T mutation installation: PEgRNAs having spacers adjacent to a NGG PAM are designed to incorporate a c.2299delG or a c. 2276 G->T mutation in wild type endogenous USH2A gene in HEK 293T cells by prime editing as a proxy to examine editing efficiency. A PE2 screen measuring editing frequency and indel formation is performed as described above at the USH2A locus corresponding to the c.2299delG or c.2276 G->T position.

Lentiviralproduction and cell line generation: Lentiviral transfer plasmids containing the USH2A c.2299delG mutation with flanking sequences from the USH2A gene on each side, and an IRES-Puromycin selection cassette, were cloned behind an EFla short promoter. HEK 293T cells were transiently transfected with the transfer plasmids and packaging plasmids containing VSV glycoprotein and lentiviral gag/pol coding sequences. After transfection, lentiviral particles were harvested from the cell media and concentrated. HEK 293T cells were transduced using serial dilutions of the lentiviral particles described above. Cells generated at a dilution of MOI <1, as determined by survival following puromycin, were selected for expansion. A resulting HEK293T cell lines carrying the c.2299delG mutation or mutations are used to screen PEgRNAs.

USH2A c.2299delG Mutation Correction with PE2 System:

PEgRNAs having spacers adjacent to a NGG PAM were designed to correct the c.2299delG mutation in HEK 293T cells by prime editing. The HEK 293T_USH2Ac.2299delG cell line as described above is expanded and transiently transfected with PEgRNA and mRNA encoding a prime editor in arrayed 96-well plates for assessment of editing by high-throughput sequencing. Prior to transfection, cells are seeded in 96-well plates and then transfected with Lipofectamine 2000 or MessengerMax according to the manufacturer's directions. Three days after transfection, gDNA is harvested in lysis buffer for high throughput sequencing and sequenced using Miseq. Indel frequency is quantified using standard quantification techniques via CRISPResso2 algorithm as described in Clement, K. et al., Nat. Biotechnol. 37, 224-226 (2019), with the quantification window defined as at least 20 bases 5′ and 3′ of PEgRNA nick site.

USH2A c.2299delG Mutation Correction with PE3 System:

PEgRNAs and ngRNAs having spacers adjacent to compatible PAMs on opposing strands are designed to correct the c.2299delG mutation in HEK 293T cells by prime editing. Chemically synthesized PEgRNAs and ngRNAs are modified at the 5′ end and the 3′end: the three 5′ most nucleotides are modified to phosphorothioated 2′-O-methyl nucleotides. The three consecutive nucleotides that precedes the 3′ most nucleotide (i.e., three consecutive nucleotides immediately 5′ of the last nucleotide at the 3′ end) are also modified to phosphorothioated 2′-O-methyl nucleotides.

The HEK 293T_USH2Ac.2299delG cell line as described above is expanded and transiently transfected with PEgRNA, ngRNA, and mRNA encoding PE2 fusion protein, in arrayed 96-well plates for assessment of editing by high-throughput sequencing. Prior to transfection, cells are seeded in 96-well plates and then transfected with Lipofectamine 2000 or MessengerMax according to the manufacturer's directions with DNA or RNA encoding a prime editor, PEgRNA, and ngRNA. Three days after transfection, gDNA is harvested in lysis buffer for high throughput sequencing and sequenced using Miseq. Indel frequency is quantified using standard quantification techniques via CRISPResso2 algorithm as described in Clement, K. et al., Nat. Biotechnol. 37, 224-226 (2019), with the quantification window defined as at least 20 bases 5′ and 3′ of pngRNA nick site.

### Example 2—Spacer Screen for Cas9 Cutting Activity Near c.2299delG and c.2276G4 T Mutation Sites in Endogenous USH2a Gene of HEK293T Cells

A spacer screen was performed to investigate Cas9 cutting activity at sites having an NGG or NGA PAM within 100 nucleotides (nts) of the c.2276 G->T mutation site in the USH2a gene. HEK293T cells were cultured and transfected with mRNA encoding a Cas9 and sgRNA as described above. The c.2276 G->T mutation site is located near the c.2299delG mutation site. Based on their location relative to these mutations, and the strand to which they bind, the indicated spacers can be used to edit one or both of these sites when used in a PEgRNA with a prime editor having a Cas9 with an inactivating mutation in the HNH domain. The mutations that can be targeted by a given spacer are listed in the Target column of Table 45. The results are shown in Table 45. A number of spacers having good cutting activity near these mutation sites were identified.

TABLE 45
Spacer screen for Cas9 cutting activity near c.2299delG and c.2276G→T
mutation sites in endogenous USH2a gene of HEK293T cells

| | | | Nick-to-edit | |
|---|---|---|---|---|
| Spacer Sequence¹ | PAM | Target² | distance (nt) | % Indel³ |
| AGGTGTGATCATTGCAATTT | TGG | 2276, 2299 | 87 | + |
| (SEQ ID NO: 138) | | | | |
| CTCCGAAGCTTTAATGATGT | TGG | 2276, 2299 | 54 | +++ |
| (SEQ ID NO: 41) | | | | |
| CCCTGCCAGTGTAACCTCCA | TGG | 2276, 2299 | 24 | ++++ |
| (SEQ ID NO: 38) | | | | |
| ACAGTCACAGGCCTTACAAT | TGG | 2299, 2276 | 99 | +++ |
| (SEQ ID NO: 5971) | | | | |
| GGTGTCACACTGAAGTCCTT | TGG | 2299, 2276 | 45 | ++++ |
| (SEQ ID NO: 3564) | | | | |
| CACTCACACTGCCCAGAGTG | AGG | 2276 | 11 | +++ |
| (SEQ ID NO: 2008) | | | | |
| AAATTTCTCCGAAGCTTTAA | TGA | 2276, 2299 | 60 | + |
| (SEQ ID NO: 31) | | | | |
| TCCGAAGCTTTAATGATGTT | GGA | 2276, 2299 | 53 | ++ |
| (SEQ ID NO: 49) | | | | |
| AGCTTTAATGATGTTGGATG | TGA | 2276, 2299 | 48 | ++++ |
| (SEQ ID NO: 34) | | | | |
| GTGTAACCTCCATGGCTCAG | TGA | 2276, 2299 | 16 | ++++ |
| (SEQ ID NO: 48) | | | | |
| GTCACAGGCCTTACAATTGG | TGA | 2299, 2276 | 96 | ++ |
| (SEQ ID NO: 5973) | | | | |
| TTTCTCTGCAGGTGTCACAC | TGA | 2299, 2276 | 55 | ++ |
| (SEQ ID NO: 380) | | | | |
| TGCACTCACACTGCCCAGAG | TGA | 2276 | 13 | ++++ |
| (SEQ ID NO: 2090) | | | | |
| ACTCACACTGCCCAGAGTGA | GGA | 2276 | 10 | ++ |
| (SEQ ID NO: 1898) | | | | |
| CTGCCCAGAGTGAGGATTGC | AGA | 2276 | 3 | + |
| (SEQ ID NO: 4519) | | | | |
| AATTCTGCAATCCTCACTCT | GGG | 2299 | 14 | +++ |
| (SEQ ID NO: 33) | | | | |
| AAATTCTGCAATCCTCACTC | TGG | 2299 | 15 | + |
| (SEQ ID NO: 30) | | | | |
| AATCCTCACTCTGGGCAGTG | TGA | 2299 | 6 | + |
| (SEQ ID NO: 32) | | | | |

¹Indicated spacer sequence was used in a synthetic sgRNA having the following scaffold: GTTTTAGAGCTAGAAATAGCAAGTTAAAATAAGGCTAGTCCGTTATCAACTTGAAAAAGTGGCACCG AGTCGGTGCTTTT (SEQ ID NO: 7543). The experimental sequence further contained 3′ mN*mN*mN*N and 5'mN*mN*mN*N modifications, where m indicates that the nucleotide contains a 2′-O-Me modification and a * indicates a phosphorothioate bond.
²The indicated spacer, when used in a PEgRNA with a Prime Editor having a Cas9 having an inactivating mutation in the HNH domain, produces a 5′ flap oriented towards the indicated mutations. The first listed mutation site is closer to the nick site; the bolded mutation site is used in the nick-to-edit distance calculation.
³+ = 0.01%-26.26%; ++ = 26.26%-55.09%; +++ = 55.09%-68.88%; ++++ = 68.88%-84.05%.
Data is average of two technical replicates.

### Example 3—Prime Editing at a Lentivirus-Introduced USH2A c.2299de1G Mutation Site in HEK293T Cells with a PEgRNA (PE2 System)

Two spacers near the c.2299delG mutation site in the USH2a gene were selected and used to design PEgRNA for correction of the c.2299delG mutation site. PEgRNA based on SEQ ID NO: 33, and compatible ngRNA for a PE3 system, are disclosed in Table 13. PEgRNA based on SEQ ID NO: 30, and compatible ngRNA for a PE3 system, are disclosed in Table 15.

PEgRNA were designed and screened in a PE2 system as described above. An HEK 293T cell line containing a lentivirus-introduced USH2A c.2299delG mutation site was generated as described above and was expanded and transiently transfected with a prime editor and synthetic PEgRNA in arrayed 96-well plates for assessment ofediting by high-throughput sequencing. Genomic DNA (gDNA) was harvested in lysis buffer for high throughput sequencing and sequenced using Miseq. Indel frequency was quantified using standard quantification techniques via CRJSPResso2 algorithm as described in Clement, K. et al., Nat. Biotechnol. 37, 224-226 (2019), with the quantification window defined as at least 20 bases 5′ and 3′ of PEgRNA nick site.

In this screen, multiple primer binding site (PBS) and reverse transcription template (RTT) lengths were tested. All the PEgRNA were designed to restore the wild-type nucleic acid sequence at the c.2299delG site. Where possible, PEgRNAs were designed to also introduce synonymous mutations that silence the PAM sequence. The results for individual PEgRNA are shown in Table 46. Successful Prime Editing was observed across PBS and RTT lengths, with and without PAM silencing.

TABLE 46
Prime Editing at a lentivirus-introduced USH2A c.2299delG mutation site in
HEK293T cells with a PEgRNA (PE2 system)

| PEgRNA¹ | | RTT² | | PBS | | | |
|---|---|---|---|---|---|---|---|
| Sequence | Spacer | Sequence | RTT | Sequence | PBS | | |
| Number | Sequence Number | Number | Length | Number | Length | % Edit³ | % Indel³ |
| 6158 | 33 | 6044* | 18 | 6018 | 10 | +++ | ++ |
| 6157 | 33 | 6043 | 18 | 6018 | 10 | +++ | ++ |
| 6173 | 33 | 6048* | 19 | 6018 | 10 | ++++ | ++ |
| 6172 | 33 | 6047 | 19 | 6018 | 10 | ++++ | ++ |
| 6194 | 33 | 6052* | 20 | 6018 | 10 | ++++ | ++ |
| 6200 | 33 | 6051 | 20 | 6018 | 10 | ++++ | ++ |
| 6221 | 33 | 6056* | 21 | 6018 | 10 | ++++ | ++ |
| 6220 | 33 | 6055 | 21 | 6018 | 10 | ++++ | ++ |
| 6251 | 33 | 6060* | 22 | 6018 | 10 | ++++ | + |
| 6261 | 33 | 6059 | 22 | 6018 | 10 | +++ | + |
| 6170 | 33 | 6044* | 18 | 6019 | 11 | ++++ | + |
| 6179 | 33 | 6043 | 18 | 6019 | 11 | +++ | + |
| 6192 | 33 | 6048* | 19 | 6019 | 11 | ++++ | + |
| 6199 | 33 | 6047 | 19 | 6019 | 11 | +++ | + |
| 6218 | 33 | 6052* | 20 | 6019 | 11 | ++++ | ++ |
| 6228 | 33 | 6051 | 20 | 6019 | 11 | ++++ | ++ |
| 6249 | 33 | 6056* | 21 | 6019 | 11 | ++++ | ++ |
| 6260 | 33 | 6055 | 21 | 6019 | 11 | +++ | ++ |
| 6289 | 33 | 6060* | 22 | 6019 | 11 | ++++ | ++ |
| 6297 | 33 | 6059 | 22 | 6019 | 11 | +++ | + |
| 6189 | 33 | 6044* | 18 | 6020 | 12 | ++++ | ++ |
| 6187 | 33 | 6043 | 18 | 6020 | 12 | +++ | + |
| 6215 | 33 | 6048* | 19 | 6020 | 12 | ++++ | + |
| 6213 | 33 | 6047 | 19 | 6020 | 12 | +++ | ++ |
| 6246 | 33 | 6052* | 20 | 6020 | 12 | ++++ | ++ |
| 6258 | 33 | 6051 | 20 | 6020 | 12 | ++++ | + |
| 6286 | 33 | 6056* | 21 | 6020 | 12 | +++ | + |
| 6284 | 33 | 6055 | 21 | 6020 | 12 | ++++ | + |
| 6323 | 33 | 6060* | 22 | 6020 | 12 | ++++ | ++ |
| 6333 | 33 | 6059 | 22 | 6020 | 12 | ++++ | ++ |
| 6209 | 33 | 6044* | 18 | 6021 | 13 | +++ | + |
| 6226 | 33 | 6043 | 18 | 6021 | 13 | +++ | + |
| 6242 | 33 | 6048* | 19 | 6021 | 13 | +++ | + |
| 6256 | 33 | 6047 | 19 | 6021 | 13 | +++ | ++ |
| 6280 | 33 | 6052* | 20 | 6021 | 13 | ++++ | + |
| 6295 | 33 | 6051 | 20 | 6021 | 13 | ++++ | + |
| 6319 | 33 | 6056* | 21 | 6021 | 13 | ++++ | ++ |
| 6331 | 33 | 6055 | 21 | 6021 | 13 | ++++ | ++ |
| 6362 | 33 | 6060* | 22 | 6021 | 13 | ++++ | ++ |
| 6372 | 33 | 6059 | 22 | 6021 | 13 | +++ | + |
| 6145 | 33 | 6044* | 18 | 6016 | 8 | +++ | ++ |
| 6144 | 33 | 6043 | 18 | 6016 | 8 | ++++ | ++ |
| 6152 | 33 | 6048* | 19 | 6016 | 8 | +++ | + |
| 6151 | 33 | 6047 | 19 | 6016 | 8 | ++++ | ++ |
| 6162 | 33 | 6052* | 20 | 6016 | 8 | +++ | + |
| 6164 | 33 | 6051 | 20 | 6016 | 8 | ++++ | + |
| 6178 | 33 | 6056* | 21 | 6016 | 8 | +++ | ++ |
| 6177 | 33 | 6055 | 21 | 6016 | 8 | +++ | + |
| 6198 | 33 | 6060* | 22 | 6016 | 8 | ++++ | ++ |
| 6202 | 33 | 6059 | 22 | 6016 | 8 | ++++ | + |
| 6149 | 33 | 6044* | 18 | 6017 | 9 | ++++ | ++ |
| 6153 | 33 | 6043 | 18 | 6017 | 9 | +++ | + |
| 6160 | 33 | 6048* | 19 | 6017 | 9 | +++ | ++ |
| 6163 | 33 | 6047 | 19 | 6017 | 9 | +++ | + |
| 6175 | 33 | 6052* | 20 | 6017 | 9 | +++ | + |
| 6180 | 33 | 6051 | 20 | 6017 | 9 | +++ | + |
| 6196 | 33 | 6056* | 21 | 6017 | 9 | +++ | + |
| 6201 | 33 | 6055 | 21 | 6017 | 9 | +++ | ++ |
| 6223 | 33 | 6060* | 22 | 6017 | 9 | +++ | + |
| 6229 | 33 | 6059 | 22 | 6017 | 9 | +++ | + |
| 7216 | 30 | 7123* | 22 | 7101 | 12 | +++ | ++ |
| 7214 | 30 | 2092 | 22 | 7101 | 12 | +++ | + |

¹Indicated PEgRNA sequence does not contain the 3′ linker and hairpin motif used experimentally. The experimental sequence further contained 3′ mN*mN*mN*N and 5′mN*mN*mN*N modifications, where m indicates that the nucleotide contains a 2′-O-Me modification and a * indicates a phosphorothioate bond.
²+ = 0.00 %-0.02%;
++ = 0.02%-0.48%;
+++ = 0.48%-1.60%;
++++ = 1.60%-15.43%.
³* = RTT encodes a PAM silencing mutation.

### Example 4—Prime Editing at a Lentivirus-Introduced USH2A c.2299delG Mutation Site in HEK293T Cells with a Combination of a PEgRNA and ngRNA (PE3 System)

PEgRNA and ngRNA were designed and screened in a PE3 system as described above. An HEK 293T cell line containing a lentivirus-introduced USH2A c.2299delG mutation site was generated as described above and was expanded and transiently transfected with a mRNA encoding a prime editor, and synthetic PEgRNA and ngRNA, in arrayed 96-well plates for assessment of editing by high-throughput sequencing. Genomic DNA (gDNA) was harvested in lysis buffer for high throughput sequencing and sequenced using Miseq. Indel frequency was quantified using standard quantification techniques via CRISPResso2 algorithm as described in Clement, K. et al., Nat. Biotechnol. 37, 224-226 (2019), with the quantification window defined as at least 20 bases 5′ and 3′ of PEgRNA nick site.

All the PEgRNA were designed to restore the wild-type nucleic acid sequence at the c.2299delG site. Where possible, PEgRNAs were designed to also introduce synonymous mutations that silence the PAM sequence. The results of the PE3 screen are provided in Table 47a; the PEgRNA used are summarized in Table 47b. Each PEgRNA was tested with three different ngRNA. Correction of the c.2299delG mutation was demonstrated with each PEgRNA tested, demonstrating that successful prime editing can be achieved across PBS and RTT lengths, both with and without PAM silencing.

TABLE 47a
Prime Editing at a lentivirus-introduced USH2A
c.2299delG mutation site in HEK293T cells
with a combination of a PEgRNA and ngRNA (PE3 system)

| PEgRNA² | ngRNA spacer² Sequence Number: | | | | | |
|---|---|---|---|---|---|---|
| Sequence | 3564 | | 4635 | | 5971 | |
| Number: | % Edit¹ | % indel¹ | % Edit¹ | % indel¹ | % Edit¹ | % indel¹ |
| 6200 | ++ | ++ | + | + | +++ | ++ |
| 6295 | ++ | +++ | +++ | ++ | +++ | ++ |
| 6160 | ++++ | ++ | ++++ | + | ++++ | + |
| 6160 | +++ | ++ | +++ | + | +++ | + |
| 6218 | ++++ | + | ++++ | + | ++++ | + |
| 6228 | +++ | +++ | +++ | ++ | ++++ | ++ |
| 6331 | +++ | +++ | ++++ | ++ | ++++ | ++ |
| 6175 | ++++ | ++ | ++++ | + | ++++ | + |
| 6249 | ++++ | ++ | ++++ | + | ++++ | + |
| 6260 | ++ | +++ | + | + | +++ | ++ |
| 6372 | +++ | +++ | +++ | ++ | ++++ | ++ |
| 6196 | ++++ | ++ | ++++ | + | +++ | + |
| 6289 | ++++ | ++ | ++++ | + | ++++ | + |
| 6297 | +++ | +++ | ++++ | + | ++++ | ++ |
| 6145 | +++ | ++ | +++ | + | +++ | ++ |
| 6189 | +++ | ++ | +++ | + | +++ | + |
| 6258 | +++ | +++ | ++++ | ++ | ++++ | ++ |
| 6162 | +++ | ++ | ++++ | + | ++++ | ++ |
| 6173 | ++++ | ++ | +++ | + | ++++ | + |
| 6246 | ++++ | ++ | ++++ | + | ++++ | + |
| 6284 | +++ | +++ | +++ | ++ | ++++ | ++ |
| 6178 | +++ | ++ | ++++ | + | ++++ | ++ |
| 6194 | ++++ | ++ | ++++ | + | ++++ | + |
| 6286 | ++++ | ++ | ++++ | ++ | +++ | + |
| 6333 | ++ | +++ | +++ | ++ | ++++ | ++ |
| 6198 | +++ | ++ | +++ | + | ++++ | ++ |
| 6221 | +++ | ++ | +++ | + | ++++ | + |
| 6280 | ++++ | + | ++++ | + | ++++ | + |
| 6226 | ++ | ++ | + | + | + | + |
| 6192 | +++ | ++ | +++ | + | +++ | + |
| 6251 | +++ | ++ | +++ | + | +++ | + |
| 6319 | ++++ | ++ | ++++ | + | +++ | + |

¹+ = 0.01%-2.73%;
++ = 2.73%-13.65%;
+++ = 13.65%-31.52%;
++++ = 31.52%-53.68%.
2Indicated sequence does not contain the 3' linker and hairpin motif used experimentally. The experimental sequence further contained 3′ mN*mN*mN*N and 5′mN*mN*mN* modifications, where m indicates that the nucleotide contains a 2′-O-Me modification and a * indicates a phosphorothioate bond. In case of ngRNA, the indicated spacer was combined with the same gRNA core used in the PEgRNA.

TABLE 47b
Summary of PEgRNA used in Table 47a

| PEgRNA³ | Spacer | RTT³ | | PBS | |
|---|---|---|---|---|---|
| Sequence | Sequence | Sequence | RTT | Sequence | PBS |
| Number: | Number: | Number: | Length | Number: | Length |
| 6200 | 33 | 6051 | 20 | 6018 | 10 |
| 6295 | 33 | 6051 | 20 | 6021 | 13 |
| 6160 | 33 | 6048* | 19 | 6017 | 9 |
| 6218 | 33 | 6052* | 20 | 6019 | 11 |
| 6228 | 33 | 6051 | 20 | 6019 | 11 |
| 6331 | 33 | 6055 | 21 | 6021 | 13 |
| 6175 | 33 | 6052* | 20 | 6017 | 9 |
| 6249 | 33 | 6056* | 21 | 6019 | 11 |
| 6260 | 33 | 6055 | 21 | 6019 | 11 |
| 6372 | 33 | 6059 | 22 | 6021 | 13 |
| 6196 | 33 | 6056* | 21 | 6017 | 9 |
| 6289 | 33 | 6060* | 22 | 6019 | 11 |
| 6297 | 33 | 6059 | 22 | 6019 | 11 |
| 6145 | 33 | 6044* | 18 | 6016 | 8 |
| 6189 | 33 | 6044* | 18 | 6020 | 12 |
| 6258 | 33 | 6051 | 20 | 6020 | 12 |
| 6162 | 33 | 6052* | 20 | 6016 | 8 |
| 6173 | 33 | 6048* | 19 | 6018 | 10 |
| 6246 | 33 | 6052* | 20 | 6020 | 12 |
| 6284 | 33 | 6055 | 21 | 6020 | 12 |
| 6178 | 33 | 6056* | 21 | 6016 | 8 |
| 6194 | 33 | 6052* | 20 | 6018 | 10 |
| 6286 | 33 | 6056* | 21 | 6020 | 12 |
| 6333 | 33 | 6059 | 22 | 6020 | 12 |
| 6198 | 33 | 6060* | 22 | 6016 | 8 |
| 6221 | 33 | 6056* | 21 | 6018 | 10 |
| 6280 | 33 | 6052* | 20 | 6021 | 13 |
| 6226 | 33 | 6043 | 18 | 6021 | 13 |
| 6192 | 33 | 6048* | 19 | 6019 | 11 |
| 6251 | 33 | 6060* | 22 | 6018 | 10 |
| 6319 | 33 | 6056* | 21 | 6021 | 13 |

³* = RTT encodes a PAM silencing mutation.

### Example 5—Prime Editing at Endogenous USH2A c.2276 GT Mutation Site in HEK293T Cells with a PEgRNA (PE2 System)

An USH2A c.2276 G->T mutation was installed at the endogenous USH2A locus in HEK 293T cells by prime editing and single-cell clones were obtained via limiting dilution and clonal expansion. A PE2 screen measuring correction and indel formation was performed at the endogenous USH2A c.2276 G->T locus. The HEK293T cells were transfected with mRNA encoding a prime editor and a synthetic PEgRNA as described in Example 1. Indel frequency was quantified using standard quantification techniques via CRISPResso2 algorithm as described in Clement, K. et al., Nat. Biotechnol. 37, 224-226 (2019), with the quantification window defined as at least 20 bases 5′ and 3′ of pngRNA and ngRNA nick site.

Five spacers were selected and used to design correction PEgRNAs having various primer biding site and reverse transcription template lengths. All the PEgRNA were designed to correct the c.2276 G->T mutation. The results for individual PEgRNA are shown in Table 48.

TABLE 48
Prime Editing at endogenous USH2A c.2276 G→T mutation
site in HEK293T cells with a PEgRNA (PE2 system)

| PEgRNA¹ | Spacer | RTT | | PBS | | | |
|---|---|---|---|---|---|---|---|
| Sequence | Sequence | Sequence | | Sequence | PBS | | |
| Number | Number | Number | RTT Length | Number | Length | % Edit² | % Indel² |
| 5885 | 38 | 5872 | 28 | 5852 | 8 | + | + |
| 5887 | 38 | 5874 | 30 | 5852 | 8 | + | + |
| 5890 | 38 | 5876 | 32 | 5852 | 8 | + | + |
| 5897 | 38 | 5878 | 34 | 5852 | 8 | + | + |
| 7515 | 38 | 5872 | 28 | 5853 | 9 | + | + |
| 7516 | 38 | 5874 | 30 | 5853 | 9 | ++ | + |
| 7517 | 38 | 5876 | 32 | 5853 | 9 | + | + |
| 7518 | 38 | 5878 | 34 | 5853 | 9 | + | + |
| 5888 | 38 | 5872 | 28 | 5856 | 12 | ++ | + |
| 5895 | 38 | 5874 | 30 | 5856 | 12 | ++ | + |
| 5899 | 38 | 5876 | 32 | 5856 | 12 | ++ | + |
| 5908 | 38 | 5878 | 34 | 5856 | 12 | ++ | + |
| 5892 | 38 | 5872 | 28 | 5858 | 14 | +++ | + |
| 5901 | 38 | 5874 | 30 | 5858 | 14 | ++ | + |
| 5905 | 38 | 5876 | 32 | 5858 | 14 | ++ | + |
| 5912 | 38 | 5878 | 34 | 5858 | 14 | ++ | + |
| 4642 | 48 | 4511 | 20 | 4529 | 8 | ++ | ++++ |
| 4659 | 48 | 4557 | 22 | 4529 | 8 | ++ | ++++ |
| 4686 | 48 | 4565 | 24 | 4529 | 8 | ++ | ++++ |
| 4750 | 48 | 4573 | 26 | 4529 | 8 | ++ | ++ |
| 4652 | 48 | 4511 | 20 | 4531 | 10 | ++ | ++++ |
| 4693 | 48 | 4557 | 22 | 4531 | 10 | ++ | ++++ |
| 4732 | 48 | 4565 | 24 | 4531 | 10 | ++ | ++++ |
| 4834 | 48 | 4573 | 26 | 4531 | 10 | + | ++++ |
| 7519 | 48 | 4511 | 20 | 4532 | 11 | ++ | ++++ |
| 7520 | 48 | 4557 | 22 | 4532 | 11 | ++ | ++++ |
| 7521 | 48 | 4565 | 24 | 4532 | 11 | ++ | ++++ |
| 7522 | 48 | 4573 | 26 | 4532 | 11 | ++ | ++++ |
| 4725 | 48 | 4511 | 20 | 4535 | 14 | ++ | ++++ |
| 4827 | 48 | 4557 | 22 | 4535 | 14 | ++ | ++++ |
| 4906 | 48 | 4565 | 24 | 4535 | 14 | ++ | ++++ |
| 5036 | 48 | 4573 | 26 | 4535 | 14 | ++ | ++++ |
| 7523 | 2090 | 2099 | 17 | 2074 | 7 | ++ | ++++ |
| 7524 | 2090 | 2107 | 19 | 2074 | 7 | +++ | ++++ |
| 7525 | 2090 | 2118 | 22 | 2074 | 7 | ++ | ++++ |
| 7526 | 2090 | 2122 | 23 | 2074 | 7 | ++ | ++++ |
| 7527 | 2090 | 2099 | 17 | 2076 | 9 | +++ | ++++ |
| 7528 | 2090 | 2107 | 19 | 2076 | 9 | +++ | ++++ |
| 7529 | 2090 | 2118 | 22 | 2076 | 9 | ++ | ++++ |
| 7530 | 2090 | 2122 | 23 | 2076 | 9 | ++ | +++ |
| 2227 | 2090 | 2099 | 17 | 2079 | 12 | +++ | ++++ |
| 2255 | 2090 | 2107 | 19 | 2079 | 12 | +++ | ++++ |
| 2342 | 2090 | 2118 | 22 | 2079 | 12 | +++ | ++++ |
| 2368 | 2090 | 2122 | 23 | 2079 | 12 | ++ | +++ |
| 7531 | 2090 | 2099 | 17 | 2080 | 13 | ++ | ++++ |
| 7532 | 2090 | 2107 | 19 | 2080 | 13 | ++ | +++ |
| 7533 | 2090 | 2118 | 22 | 2080 | 13 | ++ | +++ |
| 7534 | 2090 | 2122 | 23 | 2080 | 13 | ++ | +++ |
| 2040 | 2008 | 2014 | 15 | 1993 | 8 | +++ | + |
| 2041 | 2008 | 2016 | 17 | 1993 | 8 | +++ | + |
| 2046 | 2008 | 2019 | 20 | 1993 | 8 | +++ | + |
| 2047 | 2008 | 2020 | 21 | 1993 | 8 | +++ | + |
| 7535 | 2008 | 2014 | 15 | 1994 | 9 | +++ | + |
| 7536 | 2008 | 2016 | 17 | 1994 | 9 | ++++ | ++ |
| 7537 | 2008 | 2019 | 20 | 1994 | 9 | +++ | ++ |
| 7538 | 2008 | 2020 | 21 | 1994 | 9 | +++ | + |
| 7539 | 2008 | 2014 | 15 | 1996 | 11 | +++ | + |
| 7540 | 2008 | 2016 | 17 | 1996 | 11 | ++++ | + |
| 7541 | 2008 | 2019 | 20 | 1996 | 11 | +++ | + |
| 7542 | 2008 | 2020 | 21 | 1996 | 11 | +++ | + |
| 2050 | 2008 | 2014 | 15 | 1999 | 14 | +++ | + |
| 2054 | 2008 | 2016 | 17 | 1999 | 14 | +++ | + |
| 2063 | 2008 | 2019 | 20 | 1999 | 14 | +++ | + |
| 2065 | 2008 | 2020 | 21 | 1999 | 14 | +++ | + |
| 1958 | 1898 | 1905 | 14 | 1883 | 8 | +++ | ++++ |
| 1959 | 1898 | 1909 | 16 | 1883 | 8 | +++ | ++++ |
| 1964 | 1898 | 1915 | 19 | 1883 | 8 | +++ | ++++ |
| 1965 | 1898 | 1917 | 20 | 1883 | 8 | +++ | ++++ |
| 1960 | 1898 | 1905 | 14 | 1885 | 10 | ++ | ++++ |
| 1961 | 1898 | 1909 | 16 | 1885 | 10 | +++ | ++++ |
| 1970 | 1898 | 1915 | 19 | 1885 | 10 | +++ | ++++ |
| 1971 | 1898 | 1917 | 20 | 1885 | 10 | ++ | ++++ |
| 1962 | 1898 | 1905 | 14 | 1887 | 12 | +++ | ++++ |
| 1966 | 1898 | 1909 | 16 | 1887 | 12 | +++ | ++++ |
| 1975 | 1898 | 1915 | 19 | 1887 | 12 | +++ | ++++ |
| 1977 | 1898 | 1917 | 20 | 1887 | 12 | ++ | +++ |
| 1968 | 1898 | 1905 | 14 | 1889 | 14 | ++ | +++ |
| 1972 | 1898 | 1909 | 16 | 1889 | 14 | +++ | ++++ |
| 1981 | 1898 | 1915 | 19 | 1889 | 14 | ++ | +++ |
| 1983 | 1898 | 1917 | 20 | 1889 | 14 | + | +++ |

¹Indicated sequence does not contain the 3′ mT*mT*mT*T motif and 5'mN*mN*mN* modifications used experimentally, where m indicates that the nucleotide contains a 2′-O-Me modification, a * indicates a phosphorothioate bond, and T indicates a uracil has been added to the sequence.
²+ = 0.00%-0.24%;
++ = 0.24%-1.09%;
+++ = 1.09%-24.92%;
++++ = 24.92%-65.33%.

### Sequences and Sequence Tables

Wild-type Usherin Protein, isoform B
>sp|O75445|USH2A_HUMAN Usherin OS = Homo sapiens OX = 9606 GN = USH2A
PE = 1 SV = 3
(SEQ ID NO: 7598)
MNCPVLSLGSGFLFQVIEMLIFAYFASISLTESRGLFPRLENVGAFKKVSIVPTQAVCGL
PDRSTFCHSSAAAESIQFCTQRFCIQDCPYRSSHPTYTALFSAGLSSCITPDKNDLHPNA
HSNSASFIFGNHKSCFSSPPSPKLMASFTLAVWLKPEQQGVMCVIEKTVDGQIVFKLTIS
EKETMFYYRTVNGLQPPIKVMTLGRILVKKWIHLSVQVHQTKISFFINGVEKDHTPFNAR
TLSGSITDFASGTVQIGQSLNGLEQFVGRMQDFRLYQVALTNREILEVFSGDLLRLHAQS
HCRCPGSHPRVHPLAQRYCIPNDAGDTADNRVSRLNPEAHPLSFVNDNDVGTSWVSNVFTNITQ
LNQGVTISVDLENGQYQVFYIIIQFFSPQPTEIRIQRKKENSLDWEDWQYFARNCGAFGMKNNGD
LEKPDSVNCLQLSNFTPYSRGNVTFSILTPGPNYRPGYNNFYNTPSLQEFVKATQIRFHFHGQYY
TTETAVNLRHRYYAVDEITISGRCQCHGHADNCDTTSQPYRCLCSQESFTEGLHCDRCLPLYND
KPFRQGDQVYAFNCKPCQCNSHSKSCHYNISVDPFPFEHFRGGGGVCDDCEHNTTGRNCELCK
DYFFRQVGADPSAIDVCKPCDCDTVGTRNGSILCDQIGGQCNCKRHVSGRQCNQCQNGFYNLQ
ELDPDGCSPCNCNTSGTVDGDITCHQNSGQCKCKANVIGLRCDHCNFGFKFLRSFNDVGCEPCQ
CNLHGSVNKFCNPHSGQCECKKEAKGLQCDTCRENFYGLDVTNCKACDCDTAGSLPGTVCNA
KTGQCICKPNVEGRQCNKCLEGNFYLRQNNSFLCLPCNCDKTGTINGSLLCNKSTGQCPCKLGV
TGLRCNQCEPHRYNLTIDNFQHCQMCECDSLGTLPGTICDPISGQCLCVPNRQGRRCNQCQPGF
YISPGNATGCLPCSCHTTGAVNHICNSLTGQCVCQDASIAGQRCDQCKDHYFGFDPQTGRCQPC
NCHLSGALNETCHLVTGQCFCKQFVTGSKCDACVPSASHLDVNNLLGCSKTPFQQPPPRGQVQS
SSAINLSWSPPDSPNAHWLTYSLLRDGFEIYTTEDQYPYSIQYFLDTDLLPYTKYSYYIETTNVHG
STRSVAVTYKTKPGVPEGNLTLSYIIPIGSDSVTLTWTTLSNQSGPIEKYILSCAPLAGGQPCVSYE
GHETSATIWNLVPFAKYDFSVQACTSGGCLHSLPITVTTAQAPPQRLSPPKMQKISSTELHVEWS
PPAELNGIIIRYELYMRRLRSTKETTSEESRVFQSSGWLSPHSFVESANENALKPPQTMTTITGLEP
YTKYEFRVLAVNMAGSVSSAWVSERTGESAPVFMIPPSVFPLSSYSLNISWEKPADNVTRGKVV
GYDINMLSEQSPQQSIPMAFSQLLHTAKSQELSYTVEGLKPYRIYEFTITLCNSVGCVTSASGAGQ
TLAAAPAQLRPPLVKGINSTTIHLRWFPPEELNGPSPIYQLERRESSLPALMTTMMKGIRFIGNGY
CKFPSSTHPVNTDFTGIKASFRTKVPEGLIVFAASPGNQEEYFALQLKKGRLYFLFDPQGSPVEVT
TTNDHGKQYSDGKWHEIIAIRHQAFGQITLDGIYTGSSAILNGSTVIGDNTGVFLGGLPRSYTILR
KDPEIIQKGFVGCLKDVHFMKNYNPSAIWEPLDWQSSEEQINVYNSWEGCPASLNEGAQFLGAG
FLELHPYMFHGGMNFEISFKFRTDQLNGLLLFVYNKDGPDFLAMELKSGILTFRLNTSLAFTQVD
LLLGLSYCNGKWNKVIIKKEGSFISASVNGLMKHASESGDQPLVVNSPVYVGGIPQELLNSYQH
LCLEQGFGGCMKDVKFTRGAVVNLASVSSGAVRVNLDGCLSTDSAVNCRGNDSILVYQGKEQS
VYEGGLQPFTEYLYRVIASHEGGSVYSDWSRGRTTGAAPQSVPTPSRVRSLNGYSIEVTWDEPV
VRGVIEKYILKAYSEDSTRPPRMPSASAEFVNTSNLTGILTGLLPFKNYAVTLTACTLAGCTESSH
ALNISTPQEAPQEVQPPVAKSLPSSLLLSWNPPKKANGIITQYCLYMDGRLIYSGSEENYIVTDLA
VFTPHQFLLSACTHVGCTNSSWVLLYTAQLPPEHVDSPVLTVLDSRTIHIQWKQPRKISGILERYV
LYMSNHTHDFTIWSVIYNSTELFQDHMLQYVLPGNKYLIKLGACTGGGCTVSEASEALTDEDIPE
GVPAPKAHSYSPDSFNVSWTEPEYPNGVITSYGLYLDGILIHNSSELSYRAYGFAPWSLHSFRVQ
ACTAKGCALGPLVENRTLEAPPEGTVNVFVKTQGSRKAHVRWEAPFRPNGLLTHSVLFTGIFYV
DPVGNNYTLLNVTKVMYSGEETNLWVLIDGLVPFTNYTVQVNISNSQGSLITDPITIAMPPGAPD
GVLPPRLSSATPTSLQVVWSTPARNNAPGSPRYQLQMRSGDSTHGFLELFSNPSASLSYEVSDLQ
PYTEYMFRLVASNGFGSAHSSWIPFMTAEDKPGPVVPPILLDVKSRMMLVTWQHPRKSNGVITH
YNIYLHGRLYLRTPGNVTNCTVMHLHPYTAYKFQVEACTSKGCSLSPESQTVWTLPGAPEGIPSP
ELFSDTPTSVIISWQPPTHPNGLVENFTIERRVKGKEEVTTLVTLPRSHSMRFIDKTSALSPWTKYE
YRVLMSTLHGGTNSSAWVEVTTRPSRPAGVQPPVVTVLEPDAVQVTWKPPLIQNGDILSYEIHM
PDPHITLTNVTSAVLSQKVTHLIPFTNYSVTIVACSGGNGYLGGCTESLPTYVTTHPTVPQNVGPL
SVIPLSESYVVISWQPPSKPNGPNLRYELLRRKIQQPLASNPPEDLNRWHNIYSGTQWLYEDKGL
SRFTTYEYMLFVHNSVGFTPSREVTVTTLAGLPERGANLTASVLNHTAIDVRWAKPTVQDLQGE
VEYYTLFWSSATSNDSLKILPDVNSHVIGHLKPNTEYWIFISVFNGVHSINSAGLHATTCDGEPQG
MLPPEVVIINSTAVRVIWTSPSNPNGVVTEYSIYVNNKLYKTGMNVPGSFILRDLSPFTIYDIQVE
VCTIYACVKSNGTQITTVEDTPSDIPTPTIRGITSRSLQIDWVSPRKPNGIILGYDLLWKTWYPCAK
TQKLVQDQSDELCKAVRCQKPESICGHICYSSEAKVCCNGVLYNPKPGHRCCEEKYIPFVLNSTG
VCCGGRIQEAQPNHQCCSGYYARILPGEVCCPDEQHNRVSVGIGDSCCGRMPYSTSGNQICCAG
RLHDGHGQKCCGRQIVSNDLECCGGEEGVVYNRLPGMFCCGQDYVNMSDTICCSASSGESKAH
IKKNDPVPVKCCETELIPKSQKCCNGVGYNPLKYVCSDKISTGMMMKETKECRILCPASMEATE
HCGRCDFNFTSHICTVIRGSHNSTGKASIEEMCSSAEETIHTGSVNTYSYTDVNLKPYMTYEYRIS
AWNSYGRGLSKAVRARTKEDVPQGVSPPTWTKIDNLEDTIVLNWRKPIQSNGPIIYYILLRNGIE
RFRGTSLSFSDKEGIQPFQEYSYQLKACTVAGCATSSKVVAATTQGVPESILPPSITALSAVALHL
SWSVPEKSNGVIKEYQIRQVGKGLIHTDTTDRRQHTVTGLQPYTNYSFTLTACTSAGCTSSEPFL
GQTLQAAPEGVWVTPRHIIINSTTVELYWSLPEKPNGLVSQYQLSRNGNLLFLGGSEEQNFTDKN
LEPNSRYTYKLEVKTGGGSSASDDYIVQTPMSTPEEIYPPYNITVIGPYSIFVAWIPPGILIPEIPVEY
NVLLNDGSVTPLAFSVGHHQSTLLENLTPFTQYEIRIQACQNGSCGVSSRMFVKTPEAAPMDLNS
PVLKALGSACIEIKWMPPEKPNGIIINYFIYRRPAGIEEESVLFVWSEGALEFMDEGDTLRPFTLYE
YRVRACNSKGSVESLWSLTQTLEAPPQDFPAPWAQATSAHSVLLNWTKPESPNGIISHYRVVYQ
ERPDDPTFNSPTVHAFTVKGTSHQAHLYGLEPFTTYRIGVVAANHAGEILSPWTLIQTLESSPSGL
RNFIVEQKENGRALLLQWSEPMRTNGVIKTYNIFSDGFLEYSGLNRQFLFRRLDPFTLYTLTLEA
CTRAGCAHSAPQPLWTDEAPPDSQLAPTVHSVKSTSVELSWSEPVNPNGKIIRYEVIRRCFEGKA
WGNQTIQADEKIVFTEYNTERNTFMYNDTGLQPWTQCEYKIYTWNSAGHTCSSWNVVRTLQAP
PEGLSPPVISYVSMNPQKLLISWIPPEQSNGIIQSYRLQRNEMLYPFSFDPVTFNYTDEELLPFSTYS
YALQACTSGGCSTSKPTSITTLEAAPSEVSPPDLWAVSATQMNVCWSPPTVQNGKITKYLVRYD
NKESLAGQGLCLLVSHLQPYSQYNFSLVACTNGGCTASVSKSAWTMEALPENMDSPTLQVTGS
ESIEITWKPPRNPNGQIRSYELRRDGTIVYTGLETRYRDFTLTPGVEYSYTVTASNSQGGILSPLVK
DRTSPSAPSGMEPPKLQARGPQEILVNWDPPVRTNGDIINYTLFIRELFERETKIIHINTTHNSFGM
QSYIVNQLKPFHRYEIRIQACTTLGCASSDWTFIQTPEIAPLMQPPPHLEVQMAPGGFQPTVSLLW
TGPLQPNGKVLYYELYRRQIATQPRKSNPVLIYNGSSTSFIDSELLPFTEYEYQVWAVNSAGKAP
SSWTWCRTGPAPPEGLRAPTFHVISSTQAVVNISAPGKPNGIVSLYRLFSSSAHGAETVLSEGMA
TQQTLHGLQAFTNYSIGVEACTCFNCCSKGPTAELRTHPAPPSGLSSPQIGTLASRTASFRWSPPM
FPNGVIHSYELQFHVACPPDSALPCTPSQIETKYTGLGQKASLGGLQPYTTYKLRVVAHNEVGST
ASEWISFTTQKELPQYRAPFSVDSNLSVVCVNWSDTFLLNGQLKEYVLTDGGRRVYSGLDTTLY
IPRTADKTFFFQVICTTDEGSVKTPLIQYDTSTGLGLVLTTPGKKKGSRSKSTEFYSELWFIVLMA
MLGLILLAIFLSLILQRKIHKEPYIRERPPLVPLQKRMSPLNVYPPGENHMGLADTKIPRSGTPVSI
RSNRSACVLRIPSQNQTSLTYSQGSLHRSVSQLMDIQDKKVLMDNSLWEAIMGHNSGLYVDEED
LMNAIKDFSSVTKERTTFTDTHL.
Wild-type Usherin Protein, isoform A
(SEQ ID NO: 7608)
MNCPVLSLGSGFLFQVIEMLIFAYFASISLTESRGLFPRLENVGAFKKVSIVPTQAVCGL
PDRSTFCHSSAAAESIQFCTQRFCIQDCPYRSSHPTYTALFSAGLSSCITPDKNDLHPNA
HSNSASFIFGNHKSCFSSPPSPKLMASFTLAVWLKPEQQGVMCVIEKTVDGQIVFKLTIS
EKETMFYYRTVNGLQPPIKVMTLGRILVKKWIHLSVQVHQTKISFFINGVEKDHTPFNAR
TLSGSITDFASGTVQIGQSLNGLEQFVGRMQDERLYQVALTNREILEVFSGDLLRLHAQS
HCRCPGSHPRVHPLAQRYCIPNDAGDTADNRVSRLNPEAHPLSFVNDNDVGTSWVSNVFT
NITQLNQGVTISVDLENGQYQVFYIIIQFFSPQPTEIRIQRKKENSLDWEDWQYFARNCG
AFGMKNNGDLEKPDSVNCLQLSNFTPYSRGNVTFSILTPGPNYRPGYNNFYNTPSLQEFV
KATQIRFHFHGQYYTTETAVNLRHRYYAVDEITISGRCQCHGHADNCDTTSQPYRCLCSQ
ESFTEGLHCDRCLPLYNDKPFRQGDQVYAFNCKPCQCNSHSKSCHYNISVDPFPFEHFRG
GGGVCDDCEHNTTGRNCELCKDYFFRQVGADPSAIDVCKPCDCDTVGTRNGSILCDQIGG
QCNCKRHVSGRQCNQCQNGFYNLQELDPDGCSPCNCNTSGTVDGDITCHQNSGQCKCKAN
VIGLRCDHCNFGFKFLRSFNDVGCEPCQCNLHGSVNKFCNPHSGQCECKKEAKGLQCDTC
RENFYGLDVTNCKACDCDTAGSLPGTVCNAKTGQCICKPNVEGRQCNKCLEGNFYLRQNN
SFLCLPCNCDKTGTINGSLLCNKSTGQCPCKLGVTGLRCNQCEPHRYNLTIDNFQHCQMC
ECDSLGTLPGTICDPISGQCLCVPNRQGRRCNQCQPGFYISPGNATGCLPCSCHTTGAVN
HICNSLTGQCVCQDASIAGQRCDQCKDHYFGFDPQTGRCQPCNCHLSGALNETCHLVTGQ
CFCKQFVTGSKCDACVPSASHLDVNNLLGCSKTPFQQPPPRGQVQSSSAINLSWSPPDSP
NAHWLTYSLLRDGFEIYTTEDQYPYSIQYFLDTDLLPYTKYSYYIETTNVHGSTRSVAVT
YKTKPGVPEGNLTLSYIIPIGSDSVTLTWTTLSNQSGPIEKYILSCAPLAGGQPCVSYEG
HETSATIWNLVPFAKYDFSVQACTSGGCLHSLPITVTTAQAPPQRLSPPKMQKISSTELH
VEWSPPAELNGIIIRYELYMRRLRSTKETTSEESRVFQSSGWLSPHSFVESANENALKPP
QTMTTITGLEPYTKYEFRVLAVNMAGSVSSAWVSERTGESAPVFMIPPSVFPLSSYSLNI
SWEKPADNVTRGKVVGYDINMLSEQSPQQSIPMAFSQLLHTAKSQELSYTVEGLKPYRIY
EFTITLCNSVGCVTSASGAGQTLAAAPAQLRPPLVKGINSTTIHLRWFPPEELNGPSPIY
QLERRESSLPALMTTMMKGIRFIGNGYCKFPSSTHPVNTDFTGKCV

Lengthy table referenced here
US20240352453A1-20241024-T00001
Please refer to the end of the specification for access instructions.

Lengthy table referenced here
US20240352453A1-20241024-T00002
Please refer to the end of the specification for access instructions.

Lengthy table referenced here
US20240352453A1-20241024-T00003
Please refer to the end of the specification for access instructions.

Lengthy table referenced here
US20240352453A1-20241024-T00004
Please refer to the end of the specification for access instructions.

Lengthy table referenced here
US20240352453A1-20241024-T00005
Please refer to the end of the specification for access instructions.

Lengthy table referenced here
US20240352453A1-20241024-T00006
Please refer to the end of the specification for access instructions.

Lengthy table referenced here
US20240352453A1-20241024-T00007
Please refer to the end of the specification for access instructions.

Lengthy table referenced here
US20240352453A1-20241024-T00008
Please refer to the end of the specification for access instructions.

Lengthy table referenced here
US20240352453A1-20241024-T00009
Please refer to the end of the specification for access instructions.

Lengthy table referenced here
US20240352453A1-20241024-T00010
Please refer to the end of the specification for access instructions.

Lengthy table referenced here
US20240352453A1-20241024-T00011
Please refer to the end of the specification for access instructions.

Lengthy table referenced here
US20240352453A1-20241024-T00012
Please refer to the end of the specification for access instructions.

Lengthy table referenced here
US20240352453A1-20241024-T00013
Please refer to the end of the specification for access instructions.

Lengthy table referenced here
US20240352453A1-20241024-T00014
Please refer to the end of the specification for access instructions.

Lengthy table referenced here
US20240352453A1-20241024-T00015
Please refer to the end of the specification for access instructions.

Lengthy table referenced here
US20240352453A1-20241024-T00016
Please refer to the end of the specification for access instructions.

Lengthy table referenced here
US20240352453A1-20241024-T00017
Please refer to the end of the specification for access instructions.

Lengthy table referenced here
US20240352453A1-20241024-T00018
Please refer to the end of the specification for access instructions.

Lengthy table referenced here
US20240352453A1-20241024-T00019
Please refer to the end of the specification for access instructions.

Lengthy table referenced here
US20240352453A1-20241024-T00020
Please refer to the end of the specification for access instructions.

Lengthy table referenced here
US20240352453A1-20241024-T00021
Please refer to the end of the specification for access instructions.

Lengthy table referenced here
US20240352453A1-20241024-T00022
Please refer to the end of the specification for access instructions.

Lengthy table referenced here
US20240352453A1-20241024-T00023
Please refer to the end of the specification for access instructions.

Lengthy table referenced here
US20240352453A1-20241024-T00024
Please refer to the end of the specification for access instructions.

Lengthy table referenced here
US20240352453A1-20241024-T00025
Please refer to the end of the specification for access instructions.

Lengthy table referenced here
US20240352453A1-20241024-T00026
Please refer to the end of the specification for access instructions.

Lengthy table referenced here
US20240352453A1-20241024-T00027
Please refer to the end of the specification for access instructions.

Lengthy table referenced here
US20240352453A1-20241024-T00028
Please refer to the end of the specification for access instructions.

Lengthy table referenced here
US20240352453A1-20241024-T00029
Please refer to the end of the specification for access instructions.

Lengthy table referenced here
US20240352453A1-20241024-T00030
Please refer to the end of the specification for access instructions.

Lengthy table referenced here
US20240352453A1-20241024-T00031
Please refer to the end of the specification for access instructions.

Lengthy table referenced here
US20240352453A1-20241024-T00032
Please refer to the end of the specification for access instructions.

Lengthy table referenced here
US20240352453A1-20241024-T00033
Please refer to the end of the specification for access instructions.

Lengthy table referenced here
US20240352453A1-20241024-T00034
Please refer to the end of the specification for access instructions.

Lengthy table referenced here
US20240352453A1-20241024-T00035
Please refer to the end of the specification for access instructions.

Lengthy table referenced here
US20240352453A1-20241024-T00036
Please refer to the end of the specification for access instructions.

Lengthy table referenced here
US20240352453A1-20241024-T00037
Please refer to the end of the specification for access instructions.

Lengthy table referenced here
US20240352453A1-20241024-T00038
Please refer to the end of the specification for access instructions.

Lengthy table referenced here
US20240352453A1-20241024-T00039
Please refer to the end of the specification for access instructions.

Lengthy table referenced here
US20240352453A1-20241024-T00040
Please refer to the end of the specification for access instructions.

Lengthy table referenced here
US20240352453A1-20241024-T00041
Please refer to the end of the specification for access instructions.

Lengthy table referenced here
US20240352453A1-20241024-T00042
Please refer to the end of the specification for access instructions.

Lengthy table referenced here
US20240352453A1-20241024-T00043
Please refer to the end of the specification for access instructions.

Lengthy table referenced here
US20240352453A1-20241024-T00044
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240352453A1). An electronic copy of the table will also be available from the USPTO upon request and payment of the fee set forth in 37 CFR 1.19(b)(3).

1-98. (canceled)

99. A prime editing guide RNA (PEgRNA), or a nucleic acid encoding the PEgRNA, wherein the PEgRNA comprises:
i) a spacer comprising at its 3′ end SEQ ID NO: 602⁷,
ii) a gRNA core capable of binding to a Cas9 protein, and
iii) an extension arm comprising:
i) an editing template comprising at its 3′ end any one of SEQ ID NOs: 6032, 6033, 6038, or 6041, and
ii) a primer binding site (PBS) comprising at its 5′ end a sequence that is a reverse complement of nucleotides 11-14 of SEQ ID NO: 6027.

100. The PEgRNA of claim 99, wherein the spacer comprises at its 3′ end any one of SEQ ID NOs: 6028, 6029, 33, 6030, or 6031.

101. The PEgRNA of claim 100, wherein the spacer comprises at its 3′ end SEQ ID NO: 33.

102. The PEgRNA of claim 99, wherein the editing template comprises at its 3′ end any one of SEQ ID NOs: 6033, 6035, 6037, 6038, 6040, 6041, 6042, 6044, 6045, 6046, 6048, 6049, 6050, 6052, 6053, 6054, 6056, 6057, 6058, 6060, 6061, 6062, 6064, 6065, 6066, 6068, 6069, 6070, 6072, 6073, 6074, 6076, 6077, 6078, 6080, 6081, 6082, 6084, 6085, 6086, 6088, 6089, 6090, 6092, 6093, 6094, 6096, 6097, 6098, 6100, 6101, 6102, 6104, 6105, 6106, 6108, 6109, 6110, 6112, 6113, 6114, 6116, 6117, 6118, 6120, 6121, 6122, 6124, 6125, 6126, 6128, 6129, 6130, 6132, or 6133, and wherein the editing template encodes a PAM silencing mutation.

103. The PEgRNA of claim 102, wherein the editing temple comprises at its 3′ end any one of SEQ ID NOs: 6038, and wherein the editing template encodes a GGG-to-GCT PAM silencing edit.

104. The PEgRNA of claim 102, wherein the editing template comprises at its 3′ end SEQ ID NOs: 6033, and wherein the editing template encodes a GGG-to-GGC PAM silencing edit.

105. The PEgRNA of claim 102, wherein the editing template comprises at its 3′ end SEQ ID NO: 6041, and wherein the editing template encodes a GGG-to-GGA PAM silencing edit.

106. The PEgRNA of claim 99, wherein the editing template comprises at its 3′ end SEQ ID NO: 6032.

107. The PEgRNA of claim 99, wherein the editing template has a length of 22 nucleotides or less.

108. The PEgRNA of claim 99, wherein the PBS comprises a sequence of any one of sequence numbers: 6013, 6014, 6015, 6016, 6017, or SEQ ID NOs: 6018, 6019, 6020, 6021, 6022, 324, 6023, 6024, 6025, or 6026.

109. The PEgRNA of claim 108, wherein the PBS comprises a sequence of any one of SEQ ID NOs: 6020, 6021, or 6022.

110. The PEgRNA of claim 99, wherein the PBS is I1 to 14 nucleotides in length.

111. The PEgRNA of claim 99, wherein the spacer comprises at its 3′ end SEQ ID NO: 33, the editing template comprises at its 3′ end SEQ ID NO: 6042, and the PBS comprises at its 5′ end SEQ ID NO: 6020.

112. The PEgRNA of claim 99, wherein the spacer comprises at its 3′ end SEQ ID NO: 33, the editing template comprises at its 3′ end SEQ ID NO: 6052, and the PBS comprises at its 5′ end SEQ ID NO: 6020.

113. The PEgRNA of claim 99, wherein the spacer comprises at its 3′ end SEQ ID NO: 33, the editing template comprises at its 3′ end SEQ ID NO: 6053, and the PBS comprises at its 5′ end SEQ ID NO: 6020.

114. A prime editing system comprising
a) a prime editing guide RNA (PEgRNA) or a nucleic acid encoding the PEgRNA, wherein the PEgRNA comprises:
i) a spacer comprising at its 3′ end SEQ ID NO: 6027,
ii) a gRNA core capable of binding to a Cas9 protein, and
iii) an extension arm comprising:
i) an editing template comprising at its 3′ end any one of SEQ ID NOs: 6032, 6033, 6038, or 6041, and
ii) a primer binding site (PBS) comprising at its 5′ end a sequence that is a reverse complement of nucleotides 11-14 of SEQ ID NO: 6027; and
b) a nick guide RNA (ngRNA) or nucleic acid encoding the ngRNA, wherein the ngRNA comprises:
i) a ngRNA spacer comprising at its 3′ end a sequence corresponding to nucleotides 4-20 of any one of SEQ ID NOs: 4, 5971, 4510, 6136, 2008, 6137, 6138, 5972, 4634, 4635, 1676, 646, 4639, 3564, 5973, 1736, 4640, 2090, 380, 1098, 1324, or 5975, and
ii) a ngRNA core capable of binding a Cas9 protein.

115. The prime editing system of claim 114, wherein the ngRNA spacer comprises at its 3′end a sequence of any one of SEQ ID NOs: 5971, 6137, 2008, 3564, or 4635.

116. A method of editing an USH2a gene, the method comprising contacting the USH2a gene with:
a) a prime editing guide RNA (PEgRNA) comprising:
i) a spacer comprising at its 3′ end SEQ ID NO: 6027,
ii) a gRNA core capable of binding to a Cas9 protein, and
iii) an extension arm comprising:
a) an editing template comprising at its 3′ end any one of SEQ ID NOs: 6032, 6033, 6038, or 6041, and
b) a primer binding site (PBS) comprising at its 5′ end a sequence that is a reverse complement of nucleotides 11-14 of SEQ ID NO: 6027; and
b) a prime editor comprising the Cas9 protein and a reverse transcriptase.

117. The method of claim 116, further comprising contacting the USH2a gene with a nick guide RNA (ngRNA) comprising:
i) a ngRNA spacer comprising at its 3′ end a sequence corresponding to nucleotides 4-20 of any one of SEQ ID NOs: 4, 5971, 4510, 6136, 2008, 6137, 6138, 5972, 4634, 4635, 1676, 646, 4639, 3564, 5973, 1736, 4640, 2090, 380, 1098, 1324, or 5975, and
ii) a ngRNA core capable of binding the Cas9 protein.

118. The method of claim 116, wherein the USH2a gene is in a cell.
